(19) United States
(12) Patent Application Publication
(10) Pub. No.: US 2026/0174829 A1
(43) Pub. Date: Jun. 25, 2026

(54) METHODS OF REDUCING TYPE 2 CYTOKINE-MEDIATED INFLAMMATION USING NEUROMEDIN PEPTIDES

(71) Applicant: Rutgers, The State University of New Jersey, New Brunswick, NJ (US)

(72) Inventors: Mark C. Siracusa, Princeton, NJ (US); Jacques Y. Roberge, Princeton, NJ (US)

(73) Assignee: Rutgers, The State University of New Jersey, New Brunswick, NJ (US)

(21) Appl. No.: 19/532,862

(22) Filed: Feb. 6, 2026

Related U.S. Application Data
(63) Continuation of application No. 17/282,883.
(60) Provisional application No. 62/741,188, filed on Oct. 4, 2018.

(51) Int. Cl. A61K 38/10; A61K 9/127; A61K 31/5575; A61K 48/00; A61P 11/08; A61P 37/06; A61P 37/08

(52) U.S. Cl. CPC A61K 38/105; A61K 9/127; A61K 31/5575; A61P 11/08; A61P 37/06; A61P 37/08; A61K 48/00

(57) ABSTRACT

Methods are provided for reducing type 2 cytokine-mediated inflammation, for example by reducing IL-5 and Il-13 activity, using native and variant neuromedin B (Nmb) peptides or coding sequences. Such methods can be used to treat an inflammatory disorder, such as asthma, COPD or an allergic reaction. Also provided are modified Nmb peptides.

### CROSS REFERENCE TO RELATED APPLICATIONS

This is a Continuation of U.S. application Ser. No. 17/282,883 filed on Apr. 5, 2021, which is the U.S. National Stage of International Application No. PCT/US2019/054546 filed Oct. 3, 2019, which was published in English under PCT Article 21(2), which in turn claims the benefit of U.S. Application No. 62/741,188 filed Oct. 4, 2018, all herein incorporated by reference in their entirety.

### ACKNOWLEDGMENT OF GOVERNMENT SUPPORT

This invention was made with government support under R01 AI131634-01 awarded by the National Institutes of Health (NIH). The government has certain rights in the invention.

### FIELD

The present application provides methods of reducing type 2 cytokine-mediated inflammation, for example by reducing interleukin (IL)-5 and IL-13 activity using native and variant neuromedin peptides. In some examples, such methods are used to treat an inflammatory disorder, such as asthma or an allergic reaction.

### SEQUENCE LISTING INCORPORATION BY REFERENCE

The Sequence Listing is submitted as an XML file in the form of the file named “Sequence_Listing.xml” (89,964 bytes), which was created on Feb. 6, 2026, and is incorporated by reference herein.

### BACKGROUND

Type 2 cytokine responses characterized by the production of interleukin (IL)-4, 5, 9 and 13 promote immunity to helminth parasites, initiate tissue repair, and regulate metabolic health, but are also responsible for the inflammation associated with allergies and asthma (1-4). Cross-talk between specialized innate immune cell populations may direct the intensity of type 2 cytokine responses (5). For example, basophils provide signals to type 2 innate lymphoid cells (ILC2s), thereby promoting their production of IL-5 and IL-13 (6, 7). Additionally, the neuron-derived peptide neuromedin U (NMU) directly activates ILC2s thereby promoting immunity to Nippostrongylus brasiliensis (Nb) (8, 9). However, whether the immune and nervous systems communicate in order to limit type 2 inflammation and promote tissue integrity remains poorly defined.

### SUMMARY

It is shown herein that Nb-induced ILC2 responses are exaggerated in the absence of basophils resulting in increased inflammation and reduced lung function. Further, it is shown that ILC2s from basophil-depleted mice express reduced levels of the neuromedin B (Nmb) receptor that is associated with their enhanced activation. In vivo administration of Nmb peptides reduced infection-induced ILC2 responses, lung eosinophil and parasite clearance. Moreover, treatment with Nmb was sufficient to reduce IL-5 and IL-13 expression by sort-purified lung ILC2s from control mice but not basophil-depleted mice. Further, Nmb was also sufficient to reduce IL-5 and IL-13 expression by activated T helper type 2 (T_{H}2) T cells. Collectively, these data demonstrate basophils mediate the ability of ILC2s to respond neuron-derived signals necessary to limit inflammation and maintain tissue integrity. Further, these data also demonstrate that Nmb treatment is sufficient to reduce both innate and adaptive sources of IL-5 and IL-13 that promote type 2 inflammation.

Based on these observations, provided herein are methods of treating a disorder in a mammalian subject, such as an inflammatory disorder, for example a disorder associated with undesirable interleukin-5 (IL-5) and/or IL-13 production/activity. In some examples, such methods decrease IL-5 activity, decrease IL-13 activity, decrease ILC2 responses, decrease T_{H}2 responses, decrease eosinophilia, or combinations thereof, thereby treating the disorder. Exemplary disorders that can be treated include airway disorders (e.g., asthma, sinusitis, idiopathic pulmonary fibrosis, rhinitis, eosinophilic granulomatosis with polyangiitis, eosinophilic esophagitis, or COPD) and skin disorders (e.g., eczema, atopic dermatitis, or urticarial). Other exemplary disorders are provided in Table 1.

The method includes administering to the mammalian subject, such as a human or veterinary subject, a therapeutically effective amount of at least one neuromedin B (Nmb) protein, or at least one nucleic acid molecule encoding at least one Nmb protein, thereby treating the disorder. In some examples, the at least one Nmb protein has at least 50%, at least 60%, at least 70%, at least 80%, at least 90%, or 100% sequence identity to SEQ ID NO: 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37. In some examples, instead of using an Nmb protein, a neuromedin C (Nme) protein or a nucleic acid molecule encoding an Nme protein is used, such as a protein having at least 50%, at least 60%, at least 70%, at least 80%, at least 90%, or 100% sequence identity to SEQ ID NO: 38. In some examples, the at least one nucleic acid molecule encodes an Nmb protein having at least 50%, at least 60%, at least 70%, at least 80%, at least 90%, or 100% sequence identity to SEQ ID NO: 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37. In other examples, the nucleic acid molecule encodes an Nme protein having at least 50%, at least 60%, at least 70%, at least 80%, at least 90%, or 100% sequence identity to SEQ ID NO: 38. In some examples, at least two different Nmb proteins are used, such as (1) at least one Nmb protein having at least 50%, at least 60%, at least 70%, at least 80%, at least 90%, or 100% sequence identity to SEQ ID NO: 3, 4, 5, 6, 11, 12, 13, 14, 20, 22, 26, 27, 28, 29, 30, 31, 32, 35, 36 or 37, that is not SEQ ID NO: 1 and (2) at least one Nmb protein comprising at least 50%, at least 60%, at least 70%, at least 80%, at least 90%, or 100% sequence identity to SEQ ID NO: 26, 27, 29, 35 or 36, that is not SEQ ID NO: 1. In some examples, at least two different Nmb proteins are used, such as (1) SEQ ID NO: 1, and (2) at least one Nmb protein having at least 50%, at least 60%, at least 70%, at least 80%, at least 90%, or 100% sequence identity to SEQ ID NO: 3, 4, 5, 6, 11, 12, 13, 14, 20, 22, 26, 27, 28, 29, 30, 31, 32, 35, 36 or 37, that is not SEQ ID NO: 1. In further examples, at least one Nmb protein and at least one Nme protein are used. In some examples, the at least one nucleic acid molecule has at least 50%, at least 60%, at least 70%, at least 80%, at least 85%, at least 90%, at least 95%, at least 98%, at least 99%, or 100% sequence identity to SEQ ID NO: 2, and can be part of a plasmid or viral vector, such as a lentiviral vector or adeno-associated viral vector. In examples where a nucleic acid molecule is administered, such a nucleic acid molecule can be operably linked to a promoter, such as a constitutive promoter. In some examples, the administering comprises injection (e.g., iv, im, ip, or intradermal), oral administration, inhalational administration, or topical administration.

In some embodiments the method upregulates expression of one or more of a first set of genes comprising Sprr2a2, Serpinb2, Il1b, Xist and Tsix. In other embodiments, the method downregulates expression of one or more of a second set of genes comprising Hgs2, Nkg7, Klra7, P2rx7, Lγδc2 and Mcpt2. In further embodiments, the method upregulates expression of one or more of a first set of genes comprising Sprr2a2, Serpinb2, Il1b, Xist and Tsix and downregulates expression of one or more of a second set of genes comprising Hgs2, Nkg7, Klra7, P2rx7, Lγδc2 and Mcpt2. In other embodiments, the method decreases cellular infiltrate levels in a lung of the subject, such as a subject with an inflammatory or airway disorder.

The at least one Nmb or Nme protein or at least one nucleic acid molecule encoding at least one Nmb or Nme protein, can be present in a pharmaceutical composition, such as one that includes a pharmaceutically acceptable carrier, such as water or saline. Such compositions can include other therapeutic molecules. In some examples, at least two separate administrations of the therapeutically effective amount of the at least one Nmb or Nme protein or at least one nucleic acid molecule encoding at least one Nmb or Nme protein are provided to the subject, such as at least two separate administrations separated by at least 1 week, at least 2 weeks, at least 3 weeks, at least 4 weeks, at least 1 month, at least 2 months, at least 3 months, at least 6 months, at least 9 months, or at least one year. In some examples, the administering occurs within 5 minutes, within 10 minutes, within 30 minutes, within 1 hour, within 2 hours, within 3 hours, within 4 hours, within 5 hours, within 6 hours, within 12 hours, within 24 hours, within 48 hours, within 72 hours, within 96 hours, within 1 week, within 2 weeks, within 3 weeks, within 4 weeks, within 1 month, within 2 months, or within 3 months of the onset of the disorder. The methods can include administering to the subject a therapeutically effective amount of another therapeutic agent (e.g., see Table 1). In some examples, the additional therapeutic agent is prostaglandin E2 (PGE2).

Also provided are compositions that include an isolated Nmb protein comprising at least 50%, at least 60%, at least 70%, at least 80%, at least 85%, at least 90%, at least 95%, at least 98%, at least 99%, or 100% sequence identity to SEQ ID NO: 1 and a liposome, wherein the Nmb protein is encapsulated in the liposome. Also provided are compositions that include an isolated Nmb protein comprising at least 50%, at least 60%, at least 70%, at least 80%, at least 85%, at least 90%, at least 95%, at least 98%, at least 99%, or 100% sequence identity to SEQ ID NO: 1 and a pharmaceutically acceptable carrier. Compositions that include an isolated protein comprising at least 50%, at least 60%, at least 70%, at least 80%, at least 85%, at least 90%, at least 95%, at least 98%, at least 99%, or 100% sequence identity to SEQ ID NO: 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37, that is not SEQ ID NO: 1 or 38, and a pharmaceutically acceptable carrier, are also provided. In some examples such a composition includes a liposome, wherein the non-native Nmb protein is encapsulated in the liposome. Also provided are compositions that include (1) at least protein having at least 50%, at least 60%, at least 70%, at least 80%, at least 90%, or 100% sequence identity to SEQ ID NO: 3, 4, 5, 6, 11, 12, 13, 14, 20, 22, 26, 27, 28, 29, 30, 31, 32, 35, 36 or 37, that is not SEQ ID NO: 1 and/or (2) at least one protein comprising at least 50%, at least 60%, at least 70%, at least 80%, at least 90%, or 100% sequence identity to SEQ ID NO: 26, 27, 29, 35 or 36, that is not SEQ ID NO: 1 or 38.

The disclosed methods and compositions can also utilize fusion or chimeric proteins (or nucleic acid molecules encoding such), wherein the Nmb protein or Nme protein or non-native Nmb protein is a fusion protein that includes the Nmb protein or Nme protein or non-native Nmb protein and a cell penetrating peptide. The Nmb protein or Nme protein or non-native Nmb protein can be N- or C-terminal to the cell penetrating peptide. In addition, the Nmb protein or Nme protein or non-native Nmb protein and cell penetrating peptide can be separate by a linker.

The foregoing and other objects and features of the disclosure will become more apparent from the following detailed description, which proceeds with reference to the accompanying figures.

### BRIEF DESCRIPTION OF THE DRAWINGS

FIGS. 1A-1F. Basophils regulate helminth-induced inflammation. (A), intestinal worm burdens were quantified on day 7 post-Nb infection in control or basophil-depleted mice (baso-dep). (B), Type 2 cytokine expression in the lung of control and baso-dep mice was determined on day 7 post-Nb infection by Real-time PCR. (C), Lung-resident basophils were determined by in vivo staining with CD200R. (D), The percent of lung-resident basophils were quantified post-Nb infection. (E), Lung pathology was evaluated on day 7 post-Nb infection by hematoxylin and eosin staining. (F), Oxygen levels were determined by pulseoximeter for control and baso-dep mice post-Nb infection. P values were determined by two-tailed Student's t-tests.

FIGS. 2A-2B. Lung pathology was evaluated on day 7 post-Nb infection by (A) periodic acid shiff staining and (B) hematoxylin and eosin staining. (A), individual images were digitally tiled together to provide a larger overview.

FIGS. 3A-3H. Basophils negatively regulate ILC2 responses. (A), Neutrophils, (B) eosinophils and (C) ILC2s in the bronchoalveolar lavage (BAL) were quantified on day 7 post-Nb infection in control or baso-dep mice. Intracellular cytokine staining for (D) IL-5 and (E) IL-13 was performed on lineage negative, CD90+, CD127+ ILC2s in the BAL day 7 post-Nb infection and cytokine positive cells were quantified. Basophils were adoptively transferred (i.t.) into baso-dep mice post-Nb infection and the number of (F) ILC2s, (G) IL-5+ ILC2s and IL-13+ ILC2s, and (H) eosinophils in the BAL were quantified. P values were determined by two-tailed Student's t-tests.

FIGS. 4A-4G. (A), Neutrophils, (B) eosinophils and (C) ILC2s in the lung were quantified on day 7 post-Nb infection in control or baso-dep mice. Intracellular cytokine staining for (D) IL-5 and IL-13 was performed on lineage negative, CD90+, CD127+ ILC2s in lung on day 7 post-Nb infection and cytokine positive cells were quantified. Basophils were adoptively transferred (i.t.) into baso-dep mice post-Nb infection and the number of (E) ILC2s, (F) IL-5+ ILC2s and IL-13+ ILC2s, and (G) eosinophils in the lung were quantified. P values were determined by two-tailed Student's t-tests.

FIGS. 5A-5H. Basophils promote expression of Nmb receptor (Nmbr) on ILC2s. Rag2-deficient mice were treated with the basophil depleting antibody Ba103 and the (A) number of IL-5+ ILC2, IL-13+ ILC2 and (B) eosinophils were quantified in the BAL on day 7 post-Nb infection. Ribonucleic acid (RNA) sequencing analysis of sort-purified ILC2s from the lungs of control or basophil-depleted mice was performed on day 7 post-Nb. (C), Venn diagram illustrating genes expressed differently at 2.0-fold or higher between control or baso-dep ILC2s. DAVID pathways analysis of the 899 genes enriched in control ILC2s was performed. (D), Genes enriched in control ILC2s that define the rhodopsin-like pathway. (E) Mcpt8, (F) Nmb and (G) Nmbr expression in the lung of control or baso-dep mice was determined on day 7 post-Nb by Real-time PCR. (H) Expression of Nmbr by control or baso-dep ILC2s sort-purified from the lungs on day 7 post-Nb. P values were determined by two-tailed Student's t-tests.

FIGS. 6A-6E. Nmb suppresses type 2 cytokine responses. (A), Nb-infected mice were treated with PBS or Nmb (i.t.) and ILC2s in the BAL were quantified. (B) Expression of IL-5 and IL-13 in the lungs of PBS or Nmb-treated mice was determined on day 7 post-Nb by Real-time PCR. (C) Eosinophils in the BAL were quantified and (D) worm burdens were determined on day 7 post-Nb infection. ILC2s were sort-purified from the lungs of control or baso-dep mice on day 7 post-Nb and cultured (O/N) with IL-2 and IL-7 in the presence or absence of Nmb and (E) IL-5 and IL-13 levels in culture supernatants were determined by ELISA. P values were determined by two-tailed Student's t-tests.

FIGS. 7A-7I. Nmb treatment is sufficient to suppress allergic airway inflammation. Mice challenged with papain were treated with PBS or Nmb (i.t.) and (A) Lung ILC2s, (B) IL-5+ and IL-13+ ILC2s, and (C) eosinophils were evaluated. (B) Mesenteric lymph nodes (MLNs) were isolated on day 7 post-Nb and single cells suspensions were treated with anti-CD3 and anti-CD28 in the presence or absence of Nmb for 48 hours and (E), IL-5 and (F) IL-13 were quantified in cell-free supernatants by ELISA. (F-I), Modified versions of Nmb (see Table 2) were tested for their ability to alter IL-5 and IL-13 production from anti-CD3 and anti-CD28 MLN cells isolated on day 7 post-Nb. P values were determined by two-tailed Student's t-tests. Blue asterisks represent comparisons between media treated controls and native Nmb. Red asterisks represent comparisons between media treated controls and modified version of Nmb as described in Table 2. (*,p<0.05), (**,p<0.01), (***,p<0.001) or (F-I).

FIGS. 8A-8B. Mesenteric lymph nodes (MLNs) were isolated on day 7 post-Nb and single cells suspensions were treated with anti-CD3 and anti-CD28 in the presence or absence of native Nmb or modified versions of the peptide for 48 hours and (A), IL-5 and (B) IL-13 were quantified in cell-free supernatants by ELISA. P values were determined by two-tailed Student's t-tests. Blue asterisks represent comparisons between media treated controls and native Nmb. Red asterisks represent comparisons between media treated controls and modified version of Nmb as described in Table 2. (*,p<0.05), (**,p<0.01), (***,p<0.001).

FIG. 9. Basophils regulate NMBR expression. ILC2s were sort-purified from the lung of Nb-infected mice (day 7) and stimulated with survival cytokines (IL-7 and IL-2) and also with activated basophils, IL-4 or IL-33 overnight. NMBR expression was monitored by flow cytometric analysis post-culture. Data are presented as geographic mean fluorescent intensity (GMFI). Statistical comparisons were done using a Student's t-test.

FIGS. 10A-10B. NMB inhibits type 2 cytokine production from IL-33 activated ILC2. ILC2s were sort-purified from the lungs of Nb-infected mice (day 7) and cultured with survival cytokines (IL-2, IL-7) and in the presence or absence of Nmb and/or IL-33. Following overnight culture, supernatants were tested for the presence of IL-5 (FIG. 10A) and IL-13 (FIG. 10B) by standard ELISA. Statistical comparisons were done using a Student's t-test.

FIGS. 11A-11B. NMBR-mediated signaling on hematopoietic cells is required to regulate type 2 cytokine production. Intracellular staining for IL-5 (FIG. 11A) and IL-13 (FIG. 11B) was performed on ILC2s isolated from the BAL of naive or Nb-infected (day 7) Vav1-Cre, NMBR-floxed, or Vav1-Cre-NMBR-floxed mice. Statistical comparisons were done using a Student's t-test.

FIG. 12. NMBR-mediated signaling on hematopoietic cells is required to regulate cellular infiltrates in the lung. Lung pathology (H and E staining) was evaluated for naive or Nb-infected (day 7), Vav1-Cre, NMBR-floxed, or Vav1-Cre-NMBR-floxed mice.

FIG. 13. NMBR is expressed by several immune cell populations. Naïve (solid symbols) and Nb-infected mice (day 7) (open symbols) were evaluated for the expression of NMBR on various immune cell populations. Lymphocytes (lymphs), Alveolar macrophages (Alv Macs), Non Alveolar macrophages (NAMs), eosinophils (Eos) and Neutrophils (Neuts).

FIG. 14. Prostaglandin E2 upregulates expression of NMBR on lymphocytes. Lung ILC2s were sort-purified from the lungs of mice on day 7 post-Nb and cultured with a combination of survival cytokines (IL-2 and IL-7) and/or activating cytokines (IL-25 and IL-33). In addition, cultures were treated with NMB or prostaglandin E2 (PGE2) and the expression of NMBR was measured by flow cytometric analysis on day 4 post-culture. Statistical analysis was performed using a student's t-test. Negative NMBR staining as determined by an isotype control antibody is also shown (Isot).

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand.

SEQ ID NO: 1 is an exemplary native human Nmb protein sequence.

SEQ ID NO: 2 is an exemplary coding sequence of native human Nmb.

SEQ ID NOS: 3-37 are exemplary non-native Nmb protein sequences.

SEQ ID NO: 38 is an exemplary native human neuromedin C protein sequence.

SEQ ID NOs: 39-58 are exemplary cell penetrating peptides.

### DETAILED DESCRIPTION

Unless otherwise noted, technical terms are used according to conventional usage. Definitions of common terms in molecular biology can be found in Benjamin Lewin, Genes VII, published by Oxford University Press, 1999; Kendrew et al. (eds.), The Encyclopedia of Molecular Biology, published by Blackwell Science Ltd., 1994; and Robert A. Meyers (ed.), Molecular Biology and Biotechnology: a Comprehensive Desk Reference, published by VCH Publishers, Inc., 1995; and other similar references.

As used herein, the singular forms “a,” “an,” and “the,” refer to both the singular as well as plural, unless the context clearly indicates otherwise. As used herein, the term “comprises” means “includes.” Thus, “comprising an Nmb peptide” means “including an Nmb peptide” without excluding other elements. It is further to be understood that any and all base sizes given for nucleic acids are approximate, and are provided for descriptive purposes, unless otherwise indicated. Although many methods and materials similar or equivalent to those described herein can be used, particular suitable methods and materials are described below. In case of conflict, the present specification, including explanations of terms, will control. In addition, the materials, methods, and examples are illustrative only and not intended to be limiting. All references, including patent applications and patents, and sequences associated with the GenBank® Accession Numbers listed (as of Oct. 4, 2018) are herein incorporated by reference in their entireties.

In order to facilitate review of the various embodiments of the disclosure, the following explanations of specific terms are provided:

### I. Terms

Administration: To provide or give a subject an agent, such as a Nmb nucleic acid molecule or protein, by any effective route. Exemplary routes of administration include, but are not limited to, injection (such as intravenous, intramuscular, intradermal, intraperitoneal, or injection into the CNS, for example injection into the spine or brain), oral, nasal, transdermal, vaginal, rectal, or inhalation.

Aerosol: Any gaseous suspension of fine solid or liquid particles. As such, the term “aerosolized” refers to being in the form of microscopic solid or liquid particles dispersed or suspended in air or gas. In one example, a microscopic solid has a mass median aerodynamic diameter 1 m to 20 im. The term “nebulize” refers to the act of converting (a liquid) to a fine spray or atomizing. Accordingly, the term “dry powder aerosol” refers to any microscopic solid suspended in gas, typically air. It is also possible for the disclosed compositions (such as those that include an Nmb nucleic acid molecule or protein) to be formulated as a slow-release preparation. Aerosol delivery refers to administration (such as to the airway) of an agent (such as Nmb nucleic acid molecules or proteins) that is formulated as an aerosol.

Airway Disorder or Disease: In general, airway disorder/disease includes any disorder/disease pertaining to the airway of the lungs. In a particular non-limiting example, pulmonary disorder/disease is asthma or chronic obstructive pulmonary disease (COPD).

Allergen: Any substance that causes an allergic response or reaction. Common allergens include dust, pollen, plants, pets (e.g., cat or dog dander), medications, certain foods (e.g., eggs, cow milk, peanuts, shellfish), insect venoms, viruses or bacteria. The (detrimental) reaction may result after exposure of a subject via ingestion, inhalation, injection, or contact with skin. Any allergen can be treated using the disclosed methods.

Allergic reaction: A form of hypersensitivity (e.g., to normally innocuous entities) characterized by excessive activation of mast cells and basophils by IgE, resulting in an extreme inflammatory response. Common allergic reactions include eczema, hives, hay fever, asthma, food allergies, and reactions to the venom of stinging insects such as wasps and bees.

Amidation or amide derivative: A post-translational modification to form an amide, such as an Nmb peptide (such as any of SEQ ID NOS: 1 and 3-37) or an Nme peptide (such as SEQ ID NO: 38). Thus in some examples, the Nmb or Nme peptides provided herein are amidated, and such can be used in the disclosed methods. In amidation, the C-terminal amino acid (peptide-COOH) is modified to form and amide (peptide-CONH₂). The amide may be formed by post-translational C-terminal amidation. The amino acid to be modified can be followed by a glycine, which provides the amide group. In some examples, the amide increases the biological activity of an Nmb or Nme peptide.

Anaphylaxis: A clinical syndrome that represents the most severe systemic allergic reaction. It results from the immunologically induced release of mast cell and/or basophil mediators (including type 2 cytokines) after exposure to a specific antigen in previously sensitized individuals. Although any substance has the potential to cause anaphylaxis, the most common causes of IgE-mediated anaphylaxis are insect stings, medications, latex, peanuts and tree nuts (e.g., walnuts, hazelnuts, almonds, cashews, pecans and pistachios), shellfish and fish, milk, eggs and wheat. IgE is produced when type 2 cytokines are produced by activated ILC2s and T_{H}2 cells, thereby priming basophils and mast cells to respond to a subsequent exposure to allergens. Exercise-induced anaphylaxis and idiopathic anaphylaxis also occur, being mediated by different mechanisms. In some examples, the disclosed methods (e.g., administering a Nmb peptide) are used to treat or prevent anaphylaxis.

Asthma: Asthma is an umbrella term describing a number of syndromes or phenotypes of the respiratory system. In these disorders, the airways constrict, become inflamed, and are lined with excessive amounts of mucus, often in response to one or more “triggers,” such as exposure to an environmental stimulant (or allergen), a chemical, cold air, exercise, or viruses, or for completely unknown reasons. This airway narrowing causes symptoms such as wheezing, shortness of breath, chest tightness, and coughing. The disorder is a chronic or recurring inflammatory condition in which the airways develop increased responsiveness to the various stimuli, characterized by bronchial hyper-responsiveness, inflammation, increased mucus production, and intermittent airway obstruction. In more severely affected patients, the obstruction can become fixed as opposed to intermittent. In a typical allergic asthmatic reaction, IgE antibodies, formed by Th2 inflammatory processes, predominantly attach to mast cells that lie in the lung interstitium in close association with the bronchioles and small bronchi. Triggering of the cells causes release of several substances, including, but not limited to cytokines, chemokines and arachidonic acid derived mediators, resulting in bronchoconstriction, airway hyperreactivity, excessive mucus secretion and airway inflammation. However not all asthma is allergic and there is good evidence to suggest that patients with more severe forms of asthma have not only Th2 inflammation but Th1 inflammation present as well.

Mild asthma as defined by international guidelines as intermittent symptoms, relatively normal lung function and few exacerbations. The disclosed Nmb nucleic acid molecules and peptides can be used to treat mild asthma, for example in combination with low doses of inhaled corticosteroids (CS) or bronchodilators. Moderate asthma is defined as more persistent and severe symptoms, occasional exacerbations and/or worsening lung function. The disclosed Nmb nucleic acid molecules and peptides can be used to treat moderate asthma, for example in combination with higher doses of inhaled CSs and a long acting beta agonist (LABA) or leukotriene modifier. Severe asthma is defined in the ATS-ERS severe asthma guidelines as asthma which requires treatment with high doses of inhaled CS, in combination with a 2^{nd} controller (LABA or leukotriene modifier), or the use of systemic CSs to maintain control of disease (lower exacerbations, few symptoms and better lung function), or which never achieves control despite these treatments. The disclosed Nmb nucleic acid molecules and peptides can be used to treat severe asthma, for example in combination with such reagents.

Bronchodilator: An antispasmodic or other agent that dilates a bronchus or bronchiole. Bronchodilators relax the smooth muscles of the airways, allowing the airway to dilate. Bronchodilator medicines typically do not counteract inflammation. Examples include short (e.g., salbutamol) and long acting (e.g., formoterol) beta-2 adrenergic agonists, and anticholinergics (e.g., tiotropium and ipratropium bromide).

Cell permeability: The ability of molecules to cross a cell membrane (such as a mammalian cell). In some examples, cell permeability includes permeability through various means of transport, such as passive diffusion and transporter-mediated permeation. Certain agents can enhance cell permeability (i.e., cell permeability-enhancing agents), including peptides, such as cell-penetrating peptides, for example, as included in a tag; protein-based agents, such as pore- or channel-forming proteins; virus-based agents; lipid- or polymer-based agents; or inorganic agents. In one example, an Nmb peptide (such as any one of SEQ ID NOS: 1 and 3-37) includes a tag that increases its cell permeability, such as a peptide tag (also referred to as a cell penetrating peptide (CPP)) (e.g., transactivator of transcription, TAT, such as TAT₄₈₋₅₇, TAT₄₇₋₅₇, or TAT₄₉₋₅₇; penetratin; Pep-1; substance P, SP; polyarginines, such as R5-R12; pVEC; transportan; MAP; diatos peptide vector 1047, DPV1047, VECTOCELL®; MPG; ADP ribosylation factor, ARF, such as ARF₁₋₂₂; BPrPr (such as BPrPr₁₋₂₈); p28; VT5; Bac 7, such as Bac₁₋₂₄; C105Y; PFVYLI (SEQ ID NO: 58); Pep-7, SynB1 [RGGRLSYSRRRFSTSTGR; SEQ ID NO: 39], SynB3 [RRLSYSRRRF; SEQ ID NO: 40], PTD-4 [PIRRRKKLRRLK; SEQ ID NO: 41], PTD-5 [RRQRRTSKLMKR; SEQ ID NO: 42], FHV Coat-(35-49) [RRRRNRTRRNRRRVR; SEQ ID NO: 43], BMV Gag-(7-25) [KMTRAQRRAAARRNRWTAR; SEQ ID NO: 44], HTLV-II Rex-(4-16) [TRRQRTRRARRNR; SEQ ID NO: 45], D-Tat [GRKKRRQRRRPPQ; SEQ ID NO: 46], R9-Tat GRRRRRRRRRPPQ [SEQ ID NO: 47] and penetratin [RQIKWFQNRRMKWKK; SEQ ID NO: 48]), amphiphilic peptides (e.g., MAP [KLALKLALKLALALKLA; SEQ ID NO: 49], SBP [MGLGLHLLVLAAALQGAWSQPKKKRKV; SEQ ID NO: 50], FBP [GALFLGWLGAAGSTMGAWSQPKKKRKV; SEQ ID NO: 51], MPG ac-GALFLGFLGAAGSTMGAWSQPKKKRKV-cya; SEQ ID NO: 52], MPG(ΔNLS) [ac-GALFLGFLGAAGSTMGAWSQPKSKRKV-cya; SEQ ID NO: 53], Pep-2 [ac-KETWFETWFTEWSQPKKKRKV-cya; SEQ ID NO: 54], and transportan [GWTLNSAGYLLGKINLKALAALAKKIL; SEQ ID NO: 55]), periodic sequences (e.g., pVec, polyarginines R×N (4<N<17) chimera, polylysines K×N (4<N<17) chimera, (RAca)6R, (RAbu)6R, (RG)6R, (RM)6R, (RT)6R, (RS)6R, R10, (RA)6R, R7, and pep-1 [ac-KETWWETWWTEWSQPKKKRKV-cya; SEQ ID NO: 56]), and Cr10 (a cyclic pol-arginine CPP), or nucleic acids encoding such peptide tags.

Chimeric or fusion protein: A protein that includes a first peptide (e.g., Nmb) and a second peptide (e.g., a cell penetrating peptide), where the first and second proteins are different. A chimeric polypeptide also encompasses polypeptides that include two or more non-contiguous portions derived from the same polypeptide. In some examples, a chimeric protein includes a Nmb/cell penetrating peptide fusion protein, wherein the cell penetrating peptide is at the N- or C-terminus of Nmb. The two or more different peptides can be joined directly or indirectly, for example using a linker (such as 1-30 amino acids).

Chronic Obstructive Pulmonary Disease: A type of obstructive lung disease characterized by long-term breathing problems and poor airflow. The airflow limitation is usually progressive and associated with an abnormal inflammatory response of the lungs to noxious particles or gases. The main symptoms include shortness of breath and cough with sputum production. Tobacco smoking is the most common cause of COPD. The disclosed Nmb nucleic acid molecules and peptides can be used to treat COPD, for example in combination with a bronchodilator, corticosteroid, antibiotic, supplemental oxygen, or combinations thereof.

cDNA (complementary DNA): A piece of DNA lacking internal, non-coding segments (introns) and regulatory sequences that determine transcription. cDNA can be synthesized by reverse transcription from messenger RNA.

Contact: Placement in direct physical association, including a solid or a liquid form. Contacting can occur in vitro or ex vivo, for example, by adding a reagent to a sample (such as one containing ILC2 cells), or in vivo by administering to a subject.

Control: A reference standard. In some embodiments, the control is a healthy subject. In other embodiments, the control is a subject with a disorder listed in Table 1. In still other embodiments, the control is a historical control or standard reference value or range of values (e.g., a previously tested control subject with a known prognosis or outcome or group of subjects that represent baseline or normal values). A difference between a test subject and a control can be an increase or a decrease. The difference can be a qualitative difference or a quantitative difference, for example a statistically significant difference. In some examples, a difference is an increase or decrease, relative to a control, of at least about 5%, such as at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, at least about 500%, or greater than 500%.

Degenerate variant: A nucleotide encoding a peptide, such as Nmb, which includes a sequence that is degenerate as a result of the genetic code. There are 20 natural amino acids, most of which are specified by more than one codon. Therefore, all degenerate nucleotide sequences are included in this disclosure as long as the amino acid sequence of the peptide encoded by the nucleotide sequence is unchanged.

Effective amount or therapeutically effective amount: The amount of an agent (such as a Nmb protein or nucleic acid molecule encoding Nmb) that is sufficient to effect beneficial or desired results, for example in a subject being treated. For instance, this can be the amount of Nmb protein or nucleic acid molecule encoding Nmb necessary to reduce or suppress inflammation (such as type 2 cytokine-mediate inflammation), an asthma attack, an allergic reaction, to improve symptoms and lung function, or combinations thereof. When administered to a subject, a dosage will generally be used that will achieve target tissue concentrations (for example, in the airway) that has been shown to achieve a desired in vivo effect.

A therapeutically effective amount may vary depending upon one or more of: the subject and disease condition being treated, the weight and age of the subject, the severity of the disease condition, the manner of administration and the like, which can be determined by one of ordinary skill in the art. The beneficial therapeutic effect can include amelioration of a disease, symptom, disorder, or pathological condition; reducing or preventing the onset of a disease, symptom, disorder or condition; and generally counteracting a disease, symptom, disorder or pathological condition. In one embodiment, an “effective amount” is an amount sufficient to (1) decrease inflammation, for example in the lung or at the site of an allergic reaction, such as decrease of at least 5%, at least 10%, at least 15%, at least 20%, at least 30%, at least 40%, at least 50%, at least 60%, at least 75%, or at least 90% for example relative to no administration of the Nmb protein or nucleic acid molecule, (2) decrease IL-5, for example in ILC2 and/or T cells, such as decrease of at least 5%, at least 10%, at least 15%, at least 20%, at least 30%, at least 40%, at least 50%, at least 60%, at least 75%, or at least 90% for example relative to no administration of the Nmb protein or nucleic acid molecule, (3) decrease IL-13, for example in ILC2 and/or T cells, such as decrease of at least 5%, at least 10%, at least 15%, at least 20%, at least 30%, at least 40%, at least 50%, at least 60%, at least 75%, or at least 90% for example relative to no administration of the Nmb protein or nucleic acid molecule, (4) decrease ILC2 and/or T cell responses, for example numbers of present, proliferating and/or activated ILC2 and/or T cells, such as decrease of at least 5%, at least 10%, at least 15%, at least 20%, at least 30%, at least 40%, at least 50%, at least 60%, at least 75%, or at least 90% for example relative to no administration of the Nmb protein or nucleic acid molecule, and/or (5) decrease eosinophilia, for example in the lung or in the peripheral blood, such as decrease of at least 5%, at least 10%, at least 15%, at least 20%, at least 30%, at least 40%, at least 50%, at least 60%, at least 75%, or at least 90% for example relative to no administration of the Nmb protein or nucleic acid molecule.

Eosinophilia: A condition in which the eosinophil count in the peripheral blood exceeds about 450-500 cells/μL (for example as determined by a complete blood count (CBC)), or an increase in non-blood tissue eosinophil count observed upon histopathologic examination. Eosinophils usually account for less than 5-7% of the circulating leukocytes. Exemplary causes include allergic reaction and parasitic infection.

Expectorant: A drug or chemical substance that induces the ejection of mucus, phlegm, and other fluids from the lungs and air passages, for example by coughing. One example of such a reagent is guaifenesin.

Increase or Decrease: A statistically significant positive or negative change, respectively, in quantity from a control value (such as a value before treatment with an Nmb protein or nucleic acid molecule, or a value in a subject with a similar disorder not treated with an Nmb protein or nucleic acid molecule). An increase is a positive change, such as an increase at least 50%, at least 100%, at least 200%, at least 300%, at least 400% or at least 500% as compared to the control value. A decrease is a negative change, such as a decrease of at least 20%, at least 25%, at least 50%, at least 75%, at least 80%, at least 90%, at least 95%, at least 98%, at least 99%, or at least 100% decrease as compared to a control value. In some examples the decrease is less than 100%, such as a decrease of no more than 90%, no more than 95% or no more than 99%.

Type 2 innate lymphoid cells (ILC2s): A type of innate lymphoid cell, derived from common lymphoid progenitors. These cells lack antigen specific B or T cell receptor because of the lack of recombination activating gene. ILC2s produce type 2 cytokines (e.g., IL-4, IL-5, IL-9, IL-13).

Interleukin-5 (IL-5): (e.g., OMIM 147850): An interleukin produced by type 2 helper cells (Th2 cells), ILC2 cells, and mast cells. IL-5 stimulates B cell growth and increases immunoglobulin secretion, primarily IgA. It also mediates eosinophil activation. IL-5 is associated with the cause of several allergic diseases including allergic rhinitis and asthma. Exemplary drugs that target IL-5 include mepolizumab, benralizumab, and reslizumab, which can be used in combination with an Nmb protein or nucleic acid molecule disclosed herein, for example to treat asthma (e.g., eosinophilic asthma) or eosinophilic granulomatosis with polyangiitis (EGPA).

IL-5 sequences are publicly available, for example from the GenBank® sequence database (e.g., Accession Nos. NP_000870.1 (mature peptide aa 20-134), CAA29607.1, and NP_001006951.1 provide exemplary IL-5 protein sequences, while Accession Nos. NM_000879.2, X06271.1, and NM_001006950.1 provide exemplary IL-5 nucleic acid sequences). IL-5 variants are contemplated, such as those having at least 80%, at least 85%, at least 90%, at least 92%, at least 95%, at least 98%, or at least 99% sequence identity to such Accession Nos.

Interleukin-13 (IL-13) (e.g., OMIM 147683): A cytokine secreted by Th2 cells, CD4 cells, natural killer T cells, mast cells, basophils, eosinophils and ILC2 cells. IL-13 is a central regulator in IgE synthesis, goblet cell hyperplasia, mucus hypersecretion, airway hyperresponsiveness, fibrosis and chitinase up-regulation. It is a mediator of allergic inflammation and different diseases including asthma. Exemplary drugs that target IL-13 include tralokinumab and lebrikizumab, which can be used in combination with an Nmb protein or nucleic acid molecule disclosed herein, for example to treat asthma, atopic dermatitis, or Hodgkin's′ lymphoma.

IL-13 sequences are publicly available, for example from the GenBank® sequence database (e.g., Accession Nos. NP_002179.2 (mature peptide aa 21-132), NP_032381.1 (mature peptide aa 22-131), and NP_446280.1 (mature peptide aa 21-131) provide exemplary IL-13 protein sequences, while Accession Nos. NM_002188.2, NM_008355.3, and NM_001003384.1 provide exemplary IL-13 nucleic acid sequences). IL-13 variants are contemplated, such as those having at least 80%, at least 85%, at least 90%, at least 92%, at least 95%, at least 98%, or at least 99% sequence identity to such Accession Nos.

Inhaler: An apparatus for administering vapor or volatilized medications by inhalation. Inhalers are often used to administer medication locally to the airway, for example to treat asthma. In some examples, the inhaler is a dry powder inhaler. In other examples, the inhaler is a metered-dose inhaler. In some examples, an inhaler includes a Nmb protein disclosed herein (such as any one of SEQ ID NOS: 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37), or a nucleic acid molecule encoding such (e.g., SEQ ID NO: 2) (or a vector containing such as nucleic acid molecule).

Inhibit: To decrease, limit or block the action or function of a molecule. In an example, IL-5 and/or IL-13 activity (e.g., protein expression) is reduced or inhibited by a Nmb protein disclosed herein (such as any one of SEQ ID NOS: 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37), or a nucleic acid molecule encoding such (e.g., SEQ ID NO: 2) (or a vector containing such as nucleic acid molecule). For example, a Nmb protein disclosed herein (such as any one of SEQ ID NOS: 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37), or a nucleic acid molecule encoding such (e.g., SEQ ID NO: 2) (or a vector containing such as nucleic acid molecule), reduces or inhibits IL-5 and/or IL-13 activity by at least 10%, at least 20%, at least 40%, at least 45%, at least 50%, at least 60%, at least 75%, or at least 90% for example relative to no administration of the Nmb protein or nucleic acid molecule,

Isolated: An “isolated” biological component (such as a protein or nucleic acid, or cell) has been substantially separated, produced apart from, or purified away from other biological components in the cell or tissue of an organism in which the component occurs, such as other cells, chromosomal and extrachromosomal DNA and RNA, and proteins. Nucleic acids and proteins that have been “isolated” include nucleic acids and proteins purified by standard purification methods. The term also embraces nucleic acids and proteins (such as Nmb proteins and nucleic acid molecules) prepared by recombinant expression in a host cell as well as chemically synthesized nucleic acids and proteins. Isolated Nmb proteins, Nmb nucleic acids, or cells (such as ILC2 cells) in some examples are at least 50% pure, such as at least 75%, at least 80%, at least 90%, at least 95%, at least 98%, or at least 100% pure.

Linker or spacer: A molecule that joins together (for example, covalently joins) two or more moieties (e.g., wherein one of the moieties is an Nmb peptide) but does not have specific biological activity and does not significantly negatively affect the activity or the function of the moieties. The linker preferably is bio-compatible. The linker can be selected to provide or affect a property of the joined moieties, for example, folding, conformation, hydrophobicity, and/or spacing of the moieties. In some examples, the linker includes reactive sites at each end that each can form a covalent bond with one of the moieties included in the compounds described herein. In some examples, a linker includes a peptide, a straight or branched chain carbon linker, or a heterocyclic carbon linker.

Nebulizer: A device that turns liquid forms of medicine into a fine spray (aerosol) that can be inhaled, for example for delivering medication (such as one that includes an Nmb protein or nucleic acid) to the deep part of the respiratory tract.

Neuromedin B (Nmb): (e.g., OMIM 162340): Part of the neuromedin family of peptides including neuromedin A, B, C, K, L, N, S and U. Nmb is a bombesin-related peptide expressed in the central nervous system, lungs, gastrointestinal track and adipose tissues of mammals. Nmb acts by binding to its high affinity cell surface receptor, neuromedin B receptor (NMBR). Upon binding to its receptor, Nmb is reported to regulate cell growth, body temperature, blood pressure and glucose levels. It is shown herein that Nmb can also reduce type two cytokine-mediated inflammation, such as by reducing expression of IL-5 and IL-13.

The sequence the Nmb decapeptide is highly conserved across mammalian species: GNLWATGHFM (SEQ ID NO: 1), and can be encoded by the sequence ggcaacctctgggccaccggtcacttcatg (SEQ ID NO: 2). This decapeptide is sometimes noted as neuromedin B, or neuromedin B 23-32.

Full length Nmb sequences from with the decapeptide can be identified are publicly available, for example from the GenBank® sequence database (e.g., Accession Nos. AAA59934.1, AAH28490.1, and A37178 provide exemplary protein sequences, while Accession Nos. BC007407.2, BC028490.1, and EU375564.1 provide exemplary nucleic acid sequences). Nmb variants, such as those having at least 50%, at least 60%, at least 70%, at least 80%, at least 85%, at least 90%, at least 92%, at least 95%, at least 98%, or at least 99% sequence identity to SEQ ID NO: 1, such as any one of SEQ ID NOS: 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 and 37, can be generated and used to generate therapeutic recombinant nucleic acid molecules and proteins, for example to treat an inflammatory disorder, such as those listed in Table 1, using the methods provided herein.

Non-naturally occurring or engineered: Terms used herein as interchangeably and indicate the involvement of the hand of man. The terms, when referring to nucleic acid molecules or polypeptides (such as an Nmb molecule) indicate that the nucleic acid molecule or the polypeptide is at least substantially free from at least one other component with which they are naturally associated in nature and as found in nature. In addition, the terms can indicate that the nucleic acid molecule or polypeptide, such as a variant Nmb molecule, is one having a sequence not found in nature.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence (such as a Nmb decapeptide coding sequence). Generally, operably linked DNA sequences are contiguous and, where necessary to join two protein coding regions, in the same reading frame.

Pharmaceutically acceptable carriers: The pharmaceutically acceptable carriers of use are conventional. Remington's Pharmaceutical Sciences, by E. W. Martin, Mack Publishing Co., Easton, PA, 22nd Edition, 2013, describes compositions and formulations suitable for pharmaceutical delivery of the Nmb proteins herein disclosed.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (such as powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Polypeptide, peptide and protein: These terms are used interchangeably herein to refer to polymers of amino acids of any length, such as amino acids chemically bound together via amide linkages (CONH). The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” includes natural and/or unnatural or synthetic amino acids, including glycine and both the D or L optical isomers, and amino acid analogs and peptidomimetics. In some examples, a peptide is a Nmb peptide, such as an Nmb decapeptide.

Purified: The term purified does not require absolute purity; rather, it is intended as a relative term. Thus, for example, a purified nucleic acid is one in which the nucleic acid is more enriched than the nucleic acid in its natural environment within a cell. Similarly, a purified peptide preparation is one in which the protein is more enriched than the protein is, for example, a cell. Substantial purification denotes purification from other proteins or cellular components. In one embodiment, a preparation is purified (or isolated) such that an Nmb protein disclosed herein (such as any one of SEQ ID NOS: 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 and 37 represents at least 50% (such as, but not limited to, 70%, 80%, 90%, 95%, 98% or 99%) of the total protein content of the preparation. A Nmb protein disclosed herein (such as any one of SEQ ID NOS: 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 and 37) can be purified (and/or synthesized) by any of the means (see, e.g., Guide to Protein Purification, ed. Deutscher, Meth. Enzymol. 185, Academic Press, San Diego, 2^{nd} Edition, 2009; and Scopes, Protein Purification: Principles and Practice, Springer Verlag, New York, 3^{rd} Edition, 1994).

Sequence identity/similarity: The similarity between amino acid (or nucleotide) sequences is expressed in terms of the similarity between the sequences, otherwise referred to as sequence identity. Sequence identity is frequently measured in terms of percentage identity (or similarity or homology); the higher the percentage, the more similar the two sequences are.

Methods of alignment of sequences for comparison and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. U.S.A. 85:2444, 1988. Altschul et al., Nature Genet. 6:119, 1994, presents a detailed consideration of sequence alignment methods and homology calculations.

The NCBI Basic Local Alignment Search Tool (BLAST) (Altschul et al., J. Mol. Biol. 215:403, 1990) is available from several sources, including the National Center for Biotechnology Information (NCBI, Bethesda, MD) and on the internet, for use in connection with the sequence analysis programs blastp, blastn, blastx, tblastn and tblastx. A description of how to determine sequence identity using this program is available on the NCBI website on the internet.

Variants of protein and nucleic acid sequences (including the Nmb sequences provided herein) are typically characterized by possession of at least 50%, at least 60%, at least 70%, at least 80%, at least 90%, at least 95%, at least 96%, at least 97%, at least 98% or at least 99% sequence identity counted over the full length alignment with the amino acid sequence using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment can be performed using the Blast 2 sequences function, employing the PAM30 matrix set to default parameters (open gap 9, extension gap 1 penalties). Methods for determining sequence identity over such short windows are available at the NCBI website on the internet. These sequence identity ranges are provided for guidance only; it is entirely possible that strongly significant homologs could be obtained that fall outside of the ranges provided.

Subject: A mammal, such as a human or veterinary subject. Mammals include, but are not limited to, murines, simians, humans, farm animals, sport animals, and pets. In one embodiment, the subject is a non-human mammalian subject, such as a monkey or other non-human primate, mouse, rat, rabbit, pig, goat, sheep, dog, cat, boar, bull, horse, or cow. In some examples, the subject is a laboratory animal/organism, such as a mouse, rabbit, or rat. In some examples, the subject has an inflammatory disorder, such as one listed in Table 1 (e.g., asthma, COPD, or allergic reaction).

Treating or treatment: Refers to a therapeutic intervention that ameliorates a sign or symptom of a disease or pathological condition related to a disease or pathology (such as those listed in Table 1, such as, an airway disorder, such as asthma or chronic obstructive pulmonary disease, or an allergic reaction). Treatment can also induce remission or cure such condition. In specific examples, treatment includes reducing inflammation.

Reducing or suppressing a sign or symptom associated with a disease (such as an airway disease or other disorder listed in Table 1) can be evidenced, for example, by a delayed onset of clinical symptoms of the disease in a susceptible subject, a reduction in severity of some or all clinical symptoms of the disease, a slower progression of the disease (for example by prolonging the life of a subject having the disease), a reduction in the number of relapses of the disease, an improvement in the overall health or well-being of the subject, or by other parameters well known in the art that are specific to the particular disease.

The treatment may be assessed by objective or subjective parameters; including the results of a physical examination, and other clinical tests, and the like. In one example, treatment using the disclosed methods (1) decrease inflammation, for example in the lung or at the site of an allergic reaction, such as decrease of at least 5%, at least 10%, at least 15%, at least 20%, at least 30%, at least 40%, at least 50%, at least 60%, at least 75%, or at least 90% for example relative to no administration of the Nmb protein or nucleic acid molecule, (2) decrease IL-5, for example in ILC2 and/or T cells, such as decrease of at least 5%, at least 10%, at least 15%, at least 20%, at least 30%, at least 40%, at least 50%, at least 60%, at least 75%, or at least 90% for example relative to no administration of the Nmb protein or nucleic acid molecule, (3) decrease IL-13, for example in ILC2 and/or cells, such as decrease of at least 5%, at least 10%, at least 15%, at least 20%, at least 30%, at least 40%, at least 50%, at least 60%, at least 75%, or at least 90% for example relative to no administration of the Nmb protein or nucleic acid molecule, (4) decrease ILC2 and/or T cell responses, for example numbers of present, proliferating and/or activated ILC2 and/or T cells, such as decrease of at least 5%, at least 10%, at least 15%, at least 20%, at least 30%, at least 40%, at least 50%, at least 60%, at least 75%, or at least 90% for example relative to no administration of the Nmb protein or nucleic acid molecule, and/or (5) decrease eosinophilia, for example in the lung or in the peripheral blood, such as decrease of at least 5%, at least 10%, at least 15%, at least 20%, at least 30%, at least 40%, at least 50%, at least 60%, at least 75%, or at least 90% for example relative to no administration of the Nmb protein or nucleic acid molecule.

Under conditions sufficient for: A phrase that is used to describe any environment that permits the desired activity. In one example, this includes administering an effective amount of a composition that includes one or more Nmb proteins disclosed herein (such as one or more of SEQ ID NOS: 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 and 37), or a nucleic acid molecule encoding such (e.g., SEQ ID NO: 2) (or a vector containing such as nucleic acid molecule) sufficient to allow the desired activity (e.g., reduce inflammation associated with IL-5 and/or IL-13).

Unit dose: A physically discrete unit containing a predetermined quantity of an active material calculated to individually or collectively produce a desired effect, such as a therapeutic effect. A single unit dose or a plurality of unit doses can be used to provide the desired effect, such as treatment of a disorder, for example one listed in Table 1, such as asthma, COPD, or an allergic reaction.

Vector: A nucleic acid molecule as introduced into a cell, thereby producing a transformed cell. A vector can include nucleic acid sequences that permit it to replicate in the host cell, such as an origin of replication. A vector may also include one or more therapeutic genes (such as one encoding an Nmb peptide provided herein) and/or selectable marker genes and other genetic elements. A vector can transduce, transform or infect a cell, thereby causing the cell to express nucleic acids and/or proteins other than those native to the cell. A vector optionally includes materials to aid in achieving entry of the nucleic acid into the cell, such as a viral particle, liposome, protein coating or the like. In one example a vector is a viral vector, such as AAV or lentiviral vector.

### II. Overview

Basophils are capable of amplifying type 2 cytokines responses via their production of IL-4 and ability to promote the activation of other cell types including ILC2 and TH2 cells (2, 5, 10). Further, basophilia is a common feature of type 2 cytokine-mediated inflammation following helminth infections or the induction of allergic inflammation (2, 10). Despite basophil responses being a hallmark of type 2 inflammation, the role(s) these enigmatic cells play in regulating type 2 responses remains controversial. For example, while basophil populations rapidly expand following Nb infection, depletion of these cells has little to no effect on anti-helminth immunity and parasite clearance (2, 11). Thus, Nb-induced basophils are not contributing to parasite clearance, but may perform other unidentified functions.

The data presented herein identify a previously unappreciated aspect of cross-talk between the nervous and immune systems capable of negatively regulating ILC2 and T_{H}2 cell responses. Previous studies demonstrated that expression of neuromedin U by cholinergic neurons can promote the activation of ILC2s and amplify immunity to helminth parasites (8, 9), indicating that the nervous system and immune systems cooperate in a manner that allows for the optimal sensing and clearance of pathogens. The studies presented here indicate that as the nervous and immune systems co-evolved they also developed mechanisms to negatively regulate inflammatory responses via neuromedin B signaling in order to help maintain tissue integrity. Further, the data herein identifies an additional level of complexity and indicate that basophils promote the expression of Nmbr by immune cells allowing them to receive neuron-derived signals. Thus, other specialized innate immune cells help to mediate the lines of communication between neurons and type 2 cytokine-producing immune cells. The data further suggest that prostaglandin E2 (PGE2) promotes expression of Nmbr by lymphocytes. Since basophils are potent sources of PGE2, these data suggest that basophils regulate the ability of Nmb to inhibit inflammation via PGE2. These data bring light to the highly coordinated cellular events needed to allow for the initiation but carefully regulated persistence of inflammation at mucosal sites following exposure to an infectious agent or allergen. In addition, the data presented herein identify a previously unappreciated role of Nmb as a negative regulator of type 2 cytokines response that can be employed therapeutically to treat multiple forms of allergic disease and chronic inflammation, and other diseases.

### III. Methods of Treatment

Provided here are methods for treating a disorder in a mammalian subject, such as an inflammatory disorder, such as an inflammatory disorder caused by type 2 cytokines. In some examples, the disorder is associated with undesirable interleukin-5 (IL-5) and/or IL-13 activity. Examples of such disorders are provided in Table 1, and include allergies, airway disorders (e.g., asthma, sinusitis, idiopathic pulmonary fibrosis, rhinitis, eosinophilic granulomatosis with polyangiitis, eosinophilic esophagitis, and COPD), skin disorders (e.g., eczema, atopic dermatitis, and urticarial) and eosinophilic disorders (e.g., eosinophilic esophagitis, eosinophilic gastritis, eosinophilic gastroenteritis, eosinophilic enteritis, eosinophilic colitis, eosinophilic asthma, eosinophilic gastrointestinal disorder (EGID), eosinophilic fasciitis, EGPA, eosinophilic lung disorder, hypereosinophilic syndrome (HES), or eosinophilic leukemia). In one example, the disorder treated is Hodgkin's lymphoma. Thus, a mammalian subject, such as a human or veterinary subject, having such a disorder, can be treated with the disclosed methods.

Administering the therapeutically effective amount of one or more Nmb or Nme proteins, or one or more nucleic acid molecules encoding the one or more Nmb or Nme proteins, can (1) decrease inflammation, for example in the lung or at the site of an allergic reaction, such as decrease of at least 5%, at least 10%, at least 15%, at least 20%, at least 30%, at least 40%, at least 50%, at least 60%, at least 75%, or at least 90% for example relative to no administration of the Nmb protein or nucleic acid molecule, (2) decrease IL-5 activity (e.g., nucleic acid or protein expression), for example in ILC2 and/or cells, such as decrease of at least 5%, at least 10%, at least 15%, at least 20%, at least 30%, at least 40%, at least 50%, at least 60%, at least 75%, or at least 90% for example relative to no administration of the Nmb protein or nucleic acid molecule, (3) decrease IL-13 activity (e.g., nucleic acid or protein expression), for example in ILC2 and/or cells, such as decrease of at least 5%, at least 10%, at least 15%, at least 20%, at least 30%, at least 40%, at least 50%, at least 60%, at least 75%, or at least 90% for example relative to no administration of the Nmb protein or nucleic acid molecule, (4) decrease ILC2 and/or T cell responses, for example numbers of present, proliferating and/or activated ILC2 and/or T cells, such as decrease of at least 5%, at least 10%, at least 15%, at least 20%, at least 30%, at least 40%, at least 50%, at least 60%, at least 75%, or at least 90% for example relative to no administration of the Nmb protein or nucleic acid molecule, and/or (5) decrease eosinophilia, for example in the lung or in the peripheral blood, such as decrease of at least 5%, at least 10%, at least 15%, at least 20%, at least 30%, at least 40%, at least 50%, at least 60%, at least 75%, or at least 90% for example relative to no administration of the Nmb protein or nucleic acid molecule, thereby treating the disorder.

The disclosed methods of treatment include administering to the subject a therapeutically effective amount of at least one neuromedin B (Nmb) protein or neuromedin C (Nme) protein, or at least one nucleic acid molecule encoding at least one Nmb protein or Nme protein, thereby treating the disorder. The Nmb or Nme protein(s) or nucleic acid molecules(s) can be present in a pharmaceutical composition, such as one that includes saline or water.

Thus, in some examples, at least two different Nmb proteins are administered, such as 2, 3, 4, 5, 6, 7, 8, 9, or 10 different Nmb proteins (such as 2, 3, 4, 5, 6, 7, 8, 9, or 10 of SEQ ID NOs: 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 and 37). In some examples, at least one of the Nmb proteins administered has at least 50%, at least 60%, at least 70%, at least 80%, at least 90%, or 100% sequence identity to SEQ ID NO: 3, 4, 5, 6, 11, 12, 13, 14, 20, 22, 26, 27, 28, 29, 30, 31, 32, 35, 36 or 37. In some examples, at least one of the Nmb proteins administered has at least 50%, at least 60%, at least 70%, at least 80%, at least 90%, or 100% sequence identity to SEQ ID NO: 26, 27, 29, 35 or 36. In some examples, at least two Nmb proteins are administered, wherein (1) has at least 50%, at least 60%, at least 70%, at least 80%, at least 90%, or 100% sequence identity to SEQ ID NO: 3, 4, 5, 6, 11, 12, 13, 14, 20, 22, 26, 27, 28, 29, 30, 31, 32, 35, 36 or 37, and (2) has at least 50%, at least 60%, at least 70%, at least 80%, at least 90%, or 100% sequence identity to SEQ ID NO: 26, 27, 29, 35 or 36. In some examples, at least two Nmb proteins are administered, wherein (1) has at least 50%, at least 60%, at least 70%, at least 80%, at least 90%, or 100% sequence identity to SEQ ID NO: 3, 4, 6, 7, 9, 10, 11, 12, 14, 15, 19, 21, 22, 26, 27, 28, 29, 30, 32, 34, 35, 36, 37, and (2) has at least 50%, at least 60%, at least 70%, at least 80%, at least 90%, or 100% sequence identity to SEQ ID NO: 3, 4, 5, 6, 9, 11, 12, 13, 14, 18, 19, 20, 21, 22, 23 (wherein the two Nmb proteins are different). In some examples, at least two Nmb proteins are administered, wherein (1) comprises or consists of SEQ ID NO: 1, and (2) comprises or consists of SEQ ID NO: 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37.

In some examples, the nucleic acid molecule encoding at least one Nmb protein encodes more than one Nmb protein, such as 2, 3, 4, 5, 6, 7, 8, 9, or 10 different Nmb proteins. In some examples, the method includes administering multiple different nucleic acid molecules, each encoding at least one Nmb protein. The nucleic acid molecule(s) administered can be part of a plasmid or viral vector, such as a lentiviral or adeno-associated viral vector. In addition, the nucleic acid molecule(s) administered can be operably linked to a promoter, such as a constitutive promoter or other enhancer element.

Exemplary modes of administration include injection (e.g., iv, im, ip, or intradermal), oral administration, nasal administration, inhalational administration, or topical administration. One or more doses, such as at least two, at least 3, at least 4, or at least 5, doses of the therapeutically effective amount of the at least one Nmb protein or Nme protein or the at least one nucleic acid molecule encoding the at least one Nmb protein or Nme protein can be administered. For example, the method can include at least two separate administrations separated by at least 1 week, at least 2 weeks, at least 3 weeks, at least 4 weeks, at least 1 month, at least 2 months, at least 3 months, at least 6 months, at least 9 months, or at least one year. In some examples, the administering occurs within 5 minutes, within 10 minutes, within 30 minutes, within 1 hour, within 2 hours, within 3 hours, within 4 hours, within 5 hours, within 6 hours, within 12 hours, within 24 hours, within 48 hours, within 72 hours, within 96 hours, within 1 week, within 2 weeks, within 3 weeks, within 4 weeks, within 1 month, within 2 months, or within 3 months of the onset of the disorder.

In some examples, at least one Nmb protein administered has at least 50%, at least 60%, at least 70%, at least 80%, at least 90%, or 100% sequence identity to SEQ ID NO: 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37. In some examples, at least one nucleic acid molecule administered encodes at least one Nmb protein having at least 50%, at least 60%, at least 70%, at least 80%, at least 90%, or 100% sequence identity to SEQ ID NO: 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37. In some examples, at least one Nmb nucleic acid molecule coding sequence administered has at least 50%, at least 60%, at least 70%, at least 80%, at least 85%, at least 90%, at least 95%, at least 98%, at least 99%, or 100% sequence identity to SEQ ID NO: 2, wherein the coding sequence can be inserted or part of a vector. In some examples, a neuromedin C (Nmc) protein has at least 50%, at least 60%, at least 70%, at least 80%, at least 90%, or 100% sequence identity to SEQ ID NO: 38. In other examples, the nucleic acid molecule encodes an Nmc protein having at least 50%, at least 60%, at least 70%, at least 80%, at least 90%, or 100% sequence identity to SEQ ID NO: 38.

In some examples, the methods include administering to the subject a therapeutically effective amount of another therapeutic agent, such as one or more of those provided in Table 1. In one example, the methods include administering to the subject a therapeutically effective amount of PGE2.

TABLE 1
Exemplary Disorders and Additional Therapeutic Agents

| Disorder | Exemplary Additional Therapies |
|---|---|
| Allergies | Decongestant |
| Seasonal (e.g., pollen) | Antihistamine |
| Dust/mold | Corticosteroid |
| Food (e.g., shellfish, eggs, milk, | Immunotherapy |
| nuts, wheat) | epinephrine |
| Pets (e.g., dander) | |
| Plants | |
| Drugs (e.g., antibiotics such as sulfa | |
| or penicillin, aspirin, NSAIDs, | |
| anticonvulsants, chemotherapy | |
| drugs) | |
| Insects (e.g., cockroaches, as well as | |
| venom from honeybees, hornets, | |
| wasps, yellow jackets, fire ants) | |
| Pathogens (e.g., virus, bacterium, | |
| fungus, parasite) | |
| Latex | |
| Sinusitis | Antibiotic (e.g., amoxicillin) |
| | corticosteroid |
| Asthma (mild, moderate, or severe, | Corticosteroid |
| including eosinophilic asthma and steroid- | glucocorticoid |
| resistant asthma) | Bronchodilator (such as short (e.g., salbutamol) or |
| | long acting (e.g., formoterol) beta-2 adrenergic |
| | agonist, anticholinergic (e.g., tiotropium and |
| | ipratropium bromide) |
| | long acting beta agonist (LABA) leukotriene |
| | modifier |
| | mepolizumab |
| | benralizumab |
| | reslizumab |
| | tralokinumab |
| | lebrikizumab |
| | dupilumab |
| COPD | Corticosteroid |
| | Bronchodilator (such as short (e.g., salbutamol) or |
| | long acting (e.g., formoterol) beta-2 adrenergic |
| | agonist, anticholinergic (e.g., tiotropium and |
| | ipratropium bromide) |
| | Antibiotic (e.g., erythromycin) |
| | Supplemental oxygen |
| Idiopathic pulmonary fibrosis (IPF) | Pirfenidone |
| | Angiokinase inhibitor (e.g., Nintedanib) |
| Rhinitis | Antihistamine nasal spray |
| | Corticosteroid nasal spray |
| | Anticholingeric nasal spray (e.g., ipratropium) |
| | Decongestant (e.g., pseudoephedrine or |
| | phenylephrine) |
| eosinophilic granulomatosis with | Mepolizumab |
| polyangiitis (EGPA) | Glucocorticoid (e.g., prednisolone) |
| | Immunosuppressant (e.g., azathioprine and |
| | cyclophosphamide) |
| | Methotrexate |
| Eosinophilic Esophagitis | Topical corticosteroid (e.g., budesonide, |
| | fluticasone) |
| | Proton pump inhibitor (e.g., omeprazole, |
| | lansoprazole, dexlansoprazole, esomeprazole, |
| | pantoprazole, rabeprazole, ilaprazole) |
| Eczema | Corticosteroid (e.g., hydrocortisone, clobetasol |
| | propionate) |
| | Immunosuppressant (e.g., pimecrolimus, |
| | tacrolimus) |
| Urticaria (hives) | Antihistamine (e.g., diphenhydramine, |
| | hydroxyzine, loratadine, cetirizine, desloratadine) |
| | Leukotriene antagonist (e.g., montelukast and |
| | zafirlukast) |
| | Oral glucocorticoid |
| | Anti-inflammatory |
| | Omalizumab |
| | immunosuppressant |
| Chronic itch | Corticosteroids (e.g., cortisone and prednisone) |
| | Calcineurin inhibitors (e.g., pimecrolimus and |
| | tacrolimus) |
| | Antidepressants (e.g., Prozac and Zoloft) |
| Angioedema | Antihistamine (e.g., cetirizine) |
| | androgen |
| Conjunctivitis | Antihistamine (e.g., diphenhydramine) |
| | Mast cell stabilizer (e.g., cromolyn) |
| | Antibiotic |
| Atopic dermatitis | Tralokinumab |
| | Topical corticosteroid (e.g., hydrocortisone) |
| | Topical calcineurin inhibitor (e.g., tacrolimis or |
| | pimecrolimus) |
| | Systemic immunosuppressant (e.g., ciclospoin, |
| | methotrexate, interferon gamma-1b) |
| Eosinophilic disorders | Topical or systemic steroids (e.g., corticosteroid, |
| eosinophilic esophagitis | glucocorticoid, such as prednisone) |
| eosinophilic gastritis | Amino acid based diet |
| eosinophilic gastroenteritis | Immunosuppressant (e.g., azathioprine and |
| eosinophilic enteritis | cyclophosphamide) |
| eosinophilic colitis | For leukemia: chemotherapy (e.g., cytarabine, |
| eosinophilic asthma (see above) | anthracycline, histamine dihydrochloride, |
| eosinophilic gastrointestinal | interleukin 2), Gleevec, tyrosine kinase inhibitor |
| disorder (EGID) | (e.g., sorafenib, midostaruin, ponatinib), |
| eosinophilic Fasciitis | hematopoietic stem cell transplant |
| eosinophilic Granulomatosis with | |
| Polyangiitis, formerly Churg- | |
| Strauss Syndrome (EGPA) (see | |
| above) | |
| eosinophilic lung disorder | |
| hypereosinophilic syndrome (HES) | |
| leukemia - Eosinophilic (chronic, | |
| acute, or clonal) | |
| Parasitic or fungal infection | Antifungal agent (e.g., polyene (e.g. amphotericin |
| | B, nystatin, natamycin), azole (e.g. fluconazole, |
| | itraconazole, voriconazole), allylamine (e.g. |
| | terbinafine), and echinocandin (e.g. caspofungin) |
| | tavaborole |
| | Anti-parasitic agent (e.g., anthelmintic, |
| | antiprotozoal, antiamebic) |
| Hodgkin's lymphoma | Lebrikizumab |
| | MOPP |
| | Radiation therapy |
| | ABVD (adriamycin, bleomycin, vinblastine, |
| | dacarbazine) |
| | Stanford V (adriamycin, bleomycin, vinblastine, |
| | vincristine, chlormethine, etoposide, prednisone) |
| | BEACOPP (doxorubicine, bleomycin, vincristine, |
| | cyclophosphamide, procarbazine, etoposide, |
| | prednisone) |

### A. Neuromedin Proteins

An exemplary Nmb decapeptide is shown in SEQ ID NO: 1 (human), and variants thereof in SEQ ID NOS: 3-37. In some examples, a Nmb protein includes or consists of the protein sequence of SEQ ID NO: 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37. An exemplary Nmb coding sequence is are shown in SEQ ID NO: 2. In some examples, an Nmb nucleic acid sequence includes or consists of the sequence of SEQ ID NO: 2, which in some examples is part of a plasmid or vector, and in some examples operably linked to a promoter (such as a constitutive promoter).

In one example, the disclosed methods utilize a Nmb protein (such as a mammalian Nmb protein), that is, a Nmb protein is administered to the subject. An example of such a protein is shown in SEQ ID NO: 1 (a native Nmb protein). Native or variant Nmb proteins can be used. In one example, variant Nmb peptides are produced by manipulating a Nmb nucleotide sequence. In some examples a variant Nmb sequence is used, such as one including one or more amino acid substitutions, additions, deletions, or combinations thereof, as long as the protein retains the ability to decrease IL-5 activity, decrease IL-13 activity, decrease eosinophilia, decrease inflammation, decrease ILC2 responses, or combinations thereof. Exemplary methods of measuring IL-5 activity (e.g., measuring DNA or protein expression), IL-13 activity (e.g., measuring DNA or protein expression), eosinophilia, inflammation, and ILC2 responses, are described herein. Regions of Nmb that are more likely to tolerate substitution can be determined by aligning sequences wherein amino acids conserved between species are less likely to tolerate substitutions, while amino acids that vary at a particular position are more likely to tolerate substitutions. In addition, an alanine scan and a truncation scan can be utilized (see Example 7).

Variant Nmb proteins, such as variants of SEQ ID NO: 1, can contain one or more mutations, such as a single insertion, a single deletion, or a single amino acid substitution. In some examples, the mutant Nmb protein includes 1 to 5 insertions, 1 to 5 deletions, 1 to 5 substitutions, or any combination thereof (e.g., single insertion together with 1-5 substitutions). In some examples, the variant Nmb protein (e.g., SEQ ID NO: 1) has 1, 2, 3, 4, or 5 amino acid changes, such as 1, 2, 3, 4, or 5 amino acid substitutions (e.g., conservative amino acid substitutions), 1, 2, 3, 4, or 5 amino acid insertions, or 1, 2 or 3 amino acid deletions, or combinations thereof. In some examples, SEQ ID NO: 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37 has 1, 2, 3, 4, or 5 amino acid changes, such as 1, 2, 3, 4, or 5 amino acid insertions, 1, 2, or 3 amino acid deletions, 1, 2, 3, 4, or 5 amino acid substitutions, or any combination thereof (e.g., 1 or 2 amino acid deletions together with 1, 2, 3, 4, or 5 amino acid substitutions). In one example, SEQ ID NO: 1 includes an amino acid substitution, such as an alanine or conservative amino acid substitution, at amino acid 1, 2, 3, 4, 10. In one example, SEQ ID NO: 1 includes 2, 3, 4, or 5 amino acid substitutions, such as an alanine or conservative amino acid substitution, at amino acid 1, 2, 3, 4, and/or 10. In one example, SEQ ID NO: 1 includes 1, 2, 3, or 4 amino acid deletions, such as at amino acid 1, 2, 3, 4, 5, 6, 7, 8, 9, or 10, such as aa 5 and/or aa 7. In one example, SEQ ID NO: 1 includes 2, 3, or 4 consecutive amino acid deletions, such as at amino acids 1 and 2, 1-3, 1-4, 2-3, 2-4, 2-5 etc. In one example, SEQ ID NO: 1 includes an amino acid substitution, such as an alanine or conservative amino acid substitution, at amino acid 1, 2, 3, 4, 10, and 1 or 2 amino acid deletions at amino acid 5 and/or 7. In one example, SEQ ID NO: 1 includes a cap at the N- or C-terminus, such as a methanesulfonyl cap, NHMe, cap or OH cap.

One type of modification or mutation includes the substitution of amino acids for amino acid residues having a similar biochemical property, that is, a conservative substitution (such as 1, 2, 3, 4, or 5 conservative substitutions). Typically, conservative substitutions have little to no impact on the activity of a resulting Nmb peptide. For example, a conservative substitution is an amino acid substitution in SEQ ID NO: 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37 that does not substantially affect the ability of the Nmb peptide to decrease IL-5 activity, decrease IL-13 activity, decrease eosinophilia, decrease inflammation, decrease ILC2 responses, or combinations thereof, in a mammal. An alanine scan can be used to identify which amino acid residues in a Nmb protein, such as SEQ ID NO: 1, can tolerate an amino acid substitution (see Example 7). In one example, these activities of Nmb (e.g., SEQ ID NO: 1), are not altered by more than 25%, for example not more than 20%, for example not more than 10%, when an alanine, or other conservative amino acid, is substituted for 1, 2, 3, 4, or 5 native amino acids. Examples of amino acids which may be substituted for an original amino acid in a protein (such as Nmb) and which are regarded as conservative substitutions include: Ser for Ala; Lys for Arg; Gln or His for Asn; Glu for Asp; Ser for Cys; Asn for Gln; Asp for Glu; Pro for Gly; Asn or Gln for His; Leu or Val for Ile; Ile or Val for Leu; Arg or Gln for Lys; Leu or Ile for Met; Met, Leu or Tyr for Phe; Thr for Ser; Ser for Thr; Tyr for Trp; Trp or Phe for Tyr; and Ile or Leu for Val.

More substantial changes can be made by using substitutions that are less conservative, e.g., selecting residues that differ more significantly in their effect on maintaining: (a) the structure of the polypeptide backbone in the area of the substitution, for example, as a sheet or helical conformation; (b) the charge or hydrophobicity of the polypeptide at the target site; or (c) the bulk of the side chain. The substitutions that in general are expected to produce the greatest changes in polypeptide function are those in which: (a) a hydrophilic residue, e.g., serine or threonine, is substituted for (or by) a hydrophobic residue, e.g., leucine, isoleucine, phenylalanine, valine or alanine; (b) a cysteine or proline is substituted for (or by) any other residue; (c) a residue having an electropositive side chain, e.g., lysine, arginine, or histidine, is substituted for (or by) an electronegative residue, e.g., glutamic acid or aspartic acid; or (d) a residue having a bulky side chain, e.g., phenylalanine, is substituted for (or by) one not having a side chain, e.g., glycine. The effects of these amino acid substitutions (or other deletions or additions) can be assessed by analyzing the function of the Nmb protein, such as SEQ ID NO: 1, by analyzing the ability of the variant Nmb protein to decrease IL-5 activity, decrease IL-13 activity, decrease eosinophilia, decrease inflammation, decrease ILC2 responses, or combinations thereof, in a mammal.

In some examples, a Nmb protein used in the disclosed methods or compositions (e.g., SEQ ID NO: 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37) or a Nme protein (e.g., SEQ ID NO: 38) is PEGylated at one or more positions. In some examples, a Nmb protein or Nme protein used in the disclosed methods or compositions includes an immunoglobin FC domain. The conserved FC fragment of an antibody can be incorporated either n-terminal or c-terminal of the Nmb protein or Nme protein, and can enhance stability of the protein and therefore serum half-life. The FC domain can also be used as a means to purify the proteins on protein A or Protein G sepharose beads.

Nmb proteins (e.g., SEQ ID NO: 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37) or Nme proteins (e.g., SEQ ID NO: 38) can be tagged with cell penetrating peptide (CPP) to promote cellular uptake. Thus, in some examples, the Nmb protein or Nme protein used is a fusion protein, that includes (1) a Nmb protein or Nme protein, and (2) a cell penetrating peptide. The cell penetrating peptide can be at the N- or C-terminus of the Nmb or Nme protein. Cell penetrating peptides are usually short peptides (40 amino acids or less) that are highly cationic and usually rich in arginine and lysine that can facilitate cellular intake/uptake of proteins. Exemplary cell penetrating peptides that can be used include hydrophilic peptides (e.g., TAT [YGRKKRRQRRR; SEQ ID NO: 57], SynB1 [RGGRLSYSRRRFSTSTGR; SEQ ID NO: 39], SynB3 [RRLSYSRRRF; SEQ ID NO: 40], PTD-4 [PIRRRKKLRRLK; SEQ ID NO: 41], PTD-5 [RRQRRTSKLMKR; SEQ ID NO: 42], FHV Coat-(35-49) [RRRRNRTRRNRRRVR; SEQ ID NO: 43], BMV Gag-(7-25) [KMTRAQRRAAARRNRWTAR; SEQ ID NO: 44], HTLV-II Rex-(4-16) [TRRQRTRRARRNR; SEQ ID NO: 45], D-Tat [GRKKRRQRRRPPQ; SEQ ID NO: 46], R9-Tat GRRRRRRRRRPPQ [SEQ ID NO: 47] and penetratin [RQIKWFQNRRMKWKK; SEQ ID NO: 48]), amphiphilic peptides (e.g., MAP [KLALKLALKLALALKLA; SEQ ID NO: 49], SBP [MGLGLHLLVLAAALQGAWSQPKKKRKV; SEQ ID NO: 50], FBP [GALFLGWLGAAGSTMGAWSQPKKKRKV; SEQ ID NO: 51], MPG ac-GALFLGFLGAAGSTMGAWSQPKKKRKV-cya; SEQ ID NO: 52], MPG(ΔNLS) [ac-GALFLGFLGAAGSTMGAWSQPKSKRKV-cya; SEQ ID NO: 53], Pep-2 [ac-KETWFETWFTEWSQPKKKRKV-cya; SEQ ID NO: 54], and transportan [GWTLNSAGYLLGKINLKALAALAKKIL; SEQ ID NO: 55]), periodic sequences (e.g., pVec, polyarginines R×N (4<N<17) chimera, polylysines K×N (4<N<17) chimera, (RAca)6R, (RAbu)6R, (RG)6R, (RM)6R, (RT)6R, (RS)6R, R10, (RA)6R, R7, and pep-1 [ac-KETWWETWWTEWSQPKKKRKV-cya; SEQ ID NO: 56]), Cr10 (a cyclic pol-arginine CPP), TAT₄₈₋₅₇, TAT₄₇₋₅₇, or TAT₄₉₋₅₇; penetratin; Pep-1; substance P, SP; polyarginines, such as R5-R12; pVEC; transportan; MAP; diatos peptide vector 1047, DPV1047, VECTOCELL®; MPG; ADP ribosylation factor, ARF, such as ARF₁₋₂₂; BPrPr (such as BPrPr₁₋₂₈); p28; VT5; Bac 7, such as Bac₁₋₂₄; C105Y; PFVYLI (SEQ ID NO: 58); and Pep-7.

Nmb proteins (e.g., SEQ ID NO: 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37) or Nme proteins (e.g., SEQ ID NO: 38) can include an N-terminal cap such as formyl, acetyl, 2-18 carbons acyls, arylacyl (like benzoyl), heteroarylacyl (like 2-acetylpyridine), carbamates (like t-butylcarbamate), succinyl, alkyl or arylsulfonamide and/or a C-terminal cap, such as amide, acid, aldehyde, and esters (aryl, alkyl, heteroaryl, heteroalkyl like polyethylene glycols of 2-20 repeating units).

### B. Generation of Proteins

Isolation and purification of recombinantly expressed Nmb or Nme proteins can be carried out by conventional means, such as preparative chromatography and immunological separations. Once expressed, Nmb or Nme proteins can be purified according to standard procedures, including ammonium sulfate precipitation, affinity columns, column chromatography, and the like (see, generally, R. Scopes, Protein Purification, Springer-Verlag, N.Y., 1982). Substantially pure compositions of at least about 90 to 95% homogeneity are disclosed herein, and 98 to 99% or more homogeneity can be used for pharmaceutical purposes.

In addition to recombinant methods, Nmb or Nme proteins (or variants thereof as described above) can also be constructed in whole or in part using standard peptide synthesis. In one example, Nmb or Nme proteins are synthesized by condensation of the amino and carboxyl termini of shorter fragments. Peptide bonds can be formed by activation of a carboxyl terminal end (such as by the use of the coupling reagent N, N′-dicylohexylcarbodimide).

### C. Modifications to Proteins

Nmb peptides that can be used in the disclosed methods and compositions include synthetic embodiments of Nmb peptides described herein. In addition, analogs (non-peptide organic molecules), derivatives (chemically functionalized peptide molecules obtained starting with the disclosed peptide sequences) and variants (homologs) of these peptides can be utilized in the methods described herein. Each Nmb peptide an Nme peptide of this disclosure is comprised of a sequence of amino acids, which may be either L- and/or D-amino acids, naturally occurring and otherwise.

Nmb and Nme peptides can be modified by a variety of chemical techniques to produce derivatives having essentially the same activity as the unmodified Nmb and Nme peptides, and optionally having other desirable properties. For example, carboxylic acid groups of the peptide, whether carboxyl-terminal or side chain, can be provided in the form of a salt of a pharmaceutically-acceptable cation or esterified to form a C₁-C₁₆ ester, or converted to an amide of formula NR₁R₂ wherein R₁ and R₂ are each independently H or C₁-C₁₆ alkyl, or combined to form a heterocyclic ring, such as a 5- or 6-membered ring. Amino groups of the peptide, whether amino-terminal or side chain, can be in the form of a pharmaceutically-acceptable acid addition salt, such as the HCl, HBr, acetic, benzoic, toluene sulfonic, maleic, tartaric and other organic salts, or can be modified to C₁-C₁₆ alkyl or dialkyl amino or further converted to an amide for the incorporation of certain functionalities of linkage of ligand molecules.

Hydroxyl groups of the peptide side chains may be converted to C₁-C₁₆ alkoxy or to a C₁-C₁₆ ester to introduce hydrophobic characteristics to the peptide. Alternatively, the hydroxyl groups may be sulfated or phosphorylated to introduce negative charge and increase water solubility. Alternatively, the side chain hydroxyls or/and the asparagine and or aspartic acid side chain may be glycosylated like serine/threonine glycopeptides or N-acetylglycopeptide which can modulate the properties of the peptide. Phenyl and phenolic rings of the peptide side chains may be substituted with one or more halogen atoms, such as fluorine, chlorine, bromine or iodine, or with C1-C16 alkyl, C1-C16 alkoxy, carboxylic acids and esters thereof, or amides of such carboxylic acids. Methylene groups of the peptide side chains can be extended to homologous C2-C4 alkylenes. Thiols can be protected with any one of a number of well-recognized protecting groups, such as acetamide groups. Thiols may be reacted with maleimides or disulfides.

Peptidomimetic and organomimetic embodiments are envisioned, whereby the three-dimensional arrangement of the chemical constituents of such peptido- and organomimetics mimic the three-dimensional arrangement of the peptide backbone and component amino acid side chains, resulting in such peptido- and organomimetics of an Nmb or Nme peptide having measurable or enhanced ability to reduce type 2 cytokine production and resulting inflammation. For computer modeling applications, a pharmacophore is an idealized three-dimensional definition of the structural requirements for biological activity. Peptido- and organomimetics can be designed to fit each pharmacophore with computer modeling software (using computer assisted drug design or CADD). See Walters, “Computer-Assisted Modeling of Drugs,” in Klegerman & Groves, eds., 1993, Pharmaceutical Biotechnology, Interpharm Press: Buffalo Grove, IL, pp. 165-174 and Principles of Pharmacology, Munson (ed.) 1995, Ch. 102, for descriptions of techniques used in CADD. Also included are mimetics prepared using such techniques.

In some examples, Nmb proteins (e.g., SEQ ID NO: 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37) or Nme proteins (e.g., SEQ ID NO: 38) used in the disclosed methods or compositions include one or more modified amino acids (such as 1, 2, 3, 4 or 5 modified amino acids). Exemplary Nmb or Nme peptides are derivative peptides that may be one modified by glycosylation, pegylation, phosphorylation or any similar process that retains at least one biological function of the peptide from which it was derived (e.g., decrease IL-5 activity, decrease IL-13 activity, decrease eosinophilia, decrease inflammation, decrease ILC2 responses, or combinations thereof). Nmb or Nme peptides can also include one or more non-naturally occurring amino acids. For example, nonclassical amino acids or chemical amino acid analogs can be introduced as a substitution or addition into an Nmb peptide (e.g., SEQ ID NO: 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37) or Nme peptide (e.g., SEQ ID NO: 38). Non-classical amino acids include, but are not limited to, the D-isomers of the common amino acids, 2,4-diaminobutyric acid, alpha-amino isobutyric acid, 4-aminobutyric acid, Abu, 2-amino butyric acid, gamma-Abu, epsilon-Ahx, 6-amino hexanoic acid, Aib, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, beta-alanine, fluoro-amino acids, designer amino acids such as beta-methyl amino acids, Calpha-methyl amino acids, Nalpha-methyl amino acids, and amino acid analogs in general. Furthermore, the amino acid can be D (dextrorotary) or L (levorotary). In other specific examples, branched versions of the Nmb peptides and Nme peptides listed herein are provided, such as by substituting one or more amino acids within the sequence with an amino acid or amino acid analog with a free side chain capable of forming a peptide bond with one or more amino acids (and thus capable of forming a “branch”). Cyclical peptides are also contemplated.

Also included are peptide derivatives which are differentially modified during or after synthesis, such as by benzylation, glycosylation, acetylation, phosphorylation, amidation, pegylation, derivatization by protecting/blocking groups, proteolytic cleavage, linkage to an antibody molecule or other cellular ligand, etc. In specific embodiments, Nmb peptides (e.g., SEQ ID NO: 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37) or Nme peptides (e.g., SEQ ID NO: 38) are acetylated at the N-terminus and/or amidated at the C-terminus. In one example, an Nmb peptide (e.g., SEQ ID NO: 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37) or Nme peptide (e.g., SEQ ID NO: 38) includes a carboxy terminal amide.

Peptidomimetics are compounds based on, or derived from, peptides and proteins. Peptidomimetics can be obtained by structural modification of known peptide sequences using unnatural amino acids, conformational restraints, isosteric replacement, and the like. The subject peptidomimetics constitute the continuum of structural space between peptides and non-peptide synthetic structures; peptidomimetics may be useful, therefore, in delineating pharmacophores and in helping to translate peptides into nonpeptide compounds with the activity of the parent peptides.

Mimetopes of Nmb peptides (e.g., SEQ ID NO: 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37) or Nme peptides (e.g., SEQ ID NO: 38) are included in the present disclosure. Such peptidomimetics can have such attributes as being non-hydrolyzable (e.g., increased stability against proteases or other physiological conditions which degrade the peptide). For illustrative purposes, peptide analogs can be generated using, for example, benzodiazepines (e.g., see Freidinger et al. in Peptides: Chemistry and Biology, G. R. Marshall ed., ESCOM Publisher: Leiden, Netherlands, 1988), substituted gamma lactam rings (Garvey et al. in Peptides: Chemistry and Biology, G. R. Marshall ed., ESCOM Publisher: Leiden, Netherlands, 1988, p123), C-7 mimics (Huffman et al. in Peptides: Chemistry and Biology, G. R. Marshall ed., ESCOM Publisher: Leiden, Netherlands, 1988, p. 105), keto-methylene pseudopeptides (Ewenson et al. (1986) J Med Chem 29:295; and Ewenson et al. in Peptides: Structure and Function (Proceedings of the 9th American Peptide Symposium) Pierce Chemical Co. Rockland, Ill., 1985), R-turn dipeptide cores (Nagai et al. (1985) Tetrahedron Lett 26:647; and Sato et al. (1986) J Chem Soc Perkin Trans 1: 1231), O-aminoalcohols (Gordon et al. (1985) Biochem Biophys Res Commun 126:419; and Dann et al. (1986) Biochem Biophys Res Commun 134:71), diaminoketones (Natarajan et al. (1984) Biochem Biophys Res Commun 124:141), and methyleneamino-modified (Roark et al. in Peptides: Chemistry and Biology, G. R. Marshall ed., ESCOM Publisher: Leiden, Netherlands, 1988, p134). Also, see generally, Session III: Analytic and synthetic methods, in Peptides: Chemistry and Biology, G. R. Marshall ed., ESCOM Publisher: Leiden, Netherlands, 1988).

In addition to a variety of side-chain replacements which can be carried out to generate peptidomimetics, the present disclosure contemplates the use of conformationally restrained mimics of peptide secondary structure. Surrogates have been developed for the amide bond of peptides. Frequently exploited surrogates for the amide bond include the following groups (i) trans-olefins, (ii) fluoroalkene, (iii) methyleneamino, (iv) phosphonamides, and (v) sulfonamides. Additionally, peptidomimetics based on more substantial modifications of the backbone of a peptide can be used. Peptidomimetics which fall in this category include (i) retro-inverso analogs, and (ii) N-alkyl glycine analogs (so-called peptoids). Furthermore, the methods of combinatorial chemistry can be used to produce peptidomimetics. For example, one embodiment of a so-called “peptide morphing” strategy focuses on the random generation of a library of peptide analogs that comprise a wide range of peptide bond substitutes. In an exemplary embodiment, the peptidomimetic of an Nmb peptide (e.g., SEQ ID NO: 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37) or Nme peptide (e.g., SEQ ID NO: 38) can be derived as a retro-inverso analog of the peptide. Such retro-inverso analogs can be made according to known methods, such as that described by the Sisto et al. U.S. Pat. No. 4,522,752. A retro-inverso analog can be generated as described, for example in PCT Publication No. WO 00/01720. A mixed peptide, such as one including some normal peptide linkages, can be generated. As a general guide, sites which are most susceptible to proteolysis are typically altered, with less susceptible amide linkages being optional for mimetic switching. The final product, or intermediates thereof, can be purified.

An Nmb peptide (e.g., SEQ ID NO: 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37) or Nme peptide (e.g., SEQ ID NO: 38) can include at least one amino acid or every amino acid that is a D stereoisomer. An Nmb peptide or Nme peptide can include at least one amino acid that is reversed. The amino acid that is reversed may be a D stereoisomer. Every amino acid of a peptide may be reversed and/or every amino acid can be a D stereoisomer. In another illustrative embodiment, a peptidomimetic can be derived as a retro-enantio analog of a peptide. Retro-enantio analogs such as this can be synthesized with commercially available D-amino acids (or analogs thereof) and standard solid- or solution-phase peptide-synthesis techniques, as described, for example in PCT Publication No. WO 00/01720. The final product can be purified by HPLC to yield the pure retro-enantio analog. In still another illustrative embodiment, trans-olefin derivatives can be made for the subject peptide. Trans-olefin analogs can be synthesized according to the method of Y. K. Shue et al. (1987) Tetrahedron Letters 28:3225 and as described in PCT Publication WO 00/01720. It is further possible to couple pseudodipeptides synthesized by the above method to other pseudodipeptides, to make peptide analogs with several olefinic functionalities in place of amide functionalities. Still another class of peptidomimetic derivatives include the phosphonate derivatives. The synthesis of such phosphonate derivatives can be adapted from known synthesis schemes (see, for example, Loots et al. in Peptides: Chemistry and Biology, (Escom Science Publishers, Leiden, 1988, p. 118)); Petrillo et al. in Peptides: Structure and Function (Proceedings of the 9th American Peptide Symposium, Pierce Chemical Co. Rockland, Ill., 1985).

Other peptidomimetic structures are known and can be readily adapted for use in the subject peptidomimetics. For example, a peptidomimetic may incorporate the 1-azabicyclo[4.3.0]nonane surrogate (see Kim et al. (1997) J. Org. Chem. 62:2847), or an N-acyl piperazic acid (see Xi et al. (1998) J. Am. Chem. Soc. 120:80), or a 2-substituted piperazine moiety as a constrained amino acid analogue (see Williams et al. (1996) J. Med. Chem. 39:1345-1348). In still other embodiments, certain amino acid residues of an Nmb peptide (e.g., SEQ ID NO: 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37) or Nme peptide (e.g., SEQ ID NO: 38) can be replaced with aryl and bi-aryl moieties, such as monocyclic or bicyclic aromatic or heteroaromatic nucleus, or a biaromatic, aromatic-heteroaromatic, or biheteroaromatic nucleus. The subject peptidomimetics can be optimized such as by combinatorial synthesis techniques combined with high throughput screening. Moreover, other examples of mimetopes include, but are not limited to, protein-based compounds, carbohydrate-based compounds, lipid-based compounds, nucleic acid-based compounds, natural organic compounds, synthetically derived organic compounds, anti-idiotypic antibodies and/or catalytic antibodies, or fragments thereof. A mimetope can be obtained by, for example, screening libraries of natural and synthetic compounds for compounds capable of inhibiting fibrosis. A mimetope can also be obtained, for example, from libraries of natural and synthetic compounds, in particular, chemical or combinatorial libraries (e.g., libraries of compounds that differ in sequence or size but that have the same building blocks). A mimetope can also be obtained by, for example, rational drug design. In a rational drug design procedure, the three-dimensional structure of a compound of the present invention can be analyzed by, for example, nuclear magnetic resonance (NMR) or x-ray crystallography. The three-dimensional structure can then be used to predict structures of potential mimetopes by, for example, computer modelling. The predicted mimetope structures can then be produced by, for example, chemical synthesis, recombinant DNA technology, or by isolating a mimetope from a natural source (for example, plants, animals, bacteria and fungi).

### D. Neuromedin Nucleic Acid Molecules and Vectors

An exemplary Nmb coding sequence is shown in SEQ ID NO: 2. In some examples, an Nmb nucleic acid molecule that encodes Nmb includes or consists of the sequence of SEQ ID NO: 2. In some examples, an Nmb nucleic acid molecule encodes the protein of SEQ ID NO: 1, or a variant thereof (such as those described above). In some examples, a Nmb nucleic acid sequence includes or consists of SEQ ID NO: 2, which in some examples is part of a plasmid or vector, and in some examples operably linked to a promoter (such as a constitutive promoter).

In one example, the disclosed methods utilize a Nmb nucleic acid sequence or Nme nucleic acid sequence (such as a cDNA, genomic, or RNA sequence), that is, a Nmb or Nme nucleic acid molecule is administered to the subject, and the Nmb or Nme protein encoded expressed in the cell where the nucleic acid molecule is introduced. The Nmb or Nme nucleic acid molecule can encode a native or variant Nmb or Nme protein as described above.

Based on the genetic code, nucleic acid sequences coding for any Nmb protein (e.g., SEQ ID NO: 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37) or Nmc protein (e.g., SEQ ID NO: 38), can be generated. In some examples, such a sequence is optimized for expression in a host cell, such as a host cell used to express the Nmb or Nme protein. Such nucleic acids can be used directly (e.g., administered to a subject), or used to produce a Nmb or Nme protein which is administered to a subject.

In one example, the nucleic acid molecule encoding a Nmb protein comprises or consists of the sequence of SEQ ID NO: 2. Also provided are cells, plasmids and viral vectors including such nucleic acids, which can also include a promoter operably linked to an Nmb or Nme coding sequence.

In one example, a nucleic acid sequence the encodes for a Nmb protein has at least 50%, at least 60%, at least 70%, at least 75%, at least 80%, at least 90%, at least 92%, at least 95%, at least 96%, at least 97%, at least 99% or at least 99% sequence identity to SEQ ID NO: 2. Such sequences can readily be produced, using the amino acid sequences provided herein and the genetic code. In addition, one of skill can readily construct a variety of clones containing functionally equivalent nucleic acids, such as nucleic acids which differ in sequence but which encode the same Nmb protein sequence.

Nucleic acid molecules include DNA, cDNA, mRNA, and RNA sequences which encode a Nmb or Nme protein. Silent mutations in the coding sequence result from the degeneracy (i.e., redundancy) of the genetic code, whereby more than one codon can encode the same amino acid residue. Thus, for example, leucine can be encoded by CTT, CTC, CTA, CTG, TTA, or TTG; serine can be encoded by TCT, TCC, TCA, TCG, AGT, or AGC; asparagine can be encoded by AAT or AAC; aspartic acid can be encoded by GAT or GAC; cysteine can be encoded by TGT or TGC; alanine can be encoded by GCT, GCC, GCA, or GCG; glutamine can be encoded by CAA or CAG; tyrosine can be encoded by TAT or TAC; and isoleucine can be encoded by ATT, ATC, or ATA. Tables showing the standard genetic code can be found in various sources (see, for example, Stryer, 1988, Biochemistry, 3^{rd} Edition, W.H. 5 Freeman and Co., NY).

Codon preferences and codon usage tables for a particular species can be used to engineer isolated nucleic acid molecules encoding a Nmb protein (such as one encoding a protein having least 50%, at least 60%, at least 70%, at least 80%, at least 90%, or 100% sequence identity to SEQ ID NO: 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37) or Nme protein (such as one encoding a protein having least 50%, at least 60%, at least 70%, at least 80%, at least 90%, or 100% sequence identity to SEQ ID NO: 38) that take advantage of the codon usage preferences of that particular species. For example, the Nmb proteins used in the disclosed methods can be designed to have codons that are preferentially used by a particular organism of interest (such as a human or mouse).

A nucleic acid encoding a Nmb protein (such as a nucleic acid molecule having least 50%, at least 60%, at least 70%, at least 75%, at least 80%, at least 90%, at least 95%, at least 96%, at least 97%, at least 98%, at least 99%, or 100% sequence identity to SEQ ID NO: 2, or encoding a protein having least 50%, at least 60%, at least 70%, at least 75%, at least 80%, at least 90%, at least 95%, at least 96%, at least 97%, at least 98%, at least 99% or 100% sequence identity to SEQ ID NO: 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37) or Nme protein (such as one encoding a protein having at least 50%, at least 60%, at least 70%, at least 80%, at least 90%, at least 95%, at least 96%, at least 97%, at least 98%, at least 99%, or 100% sequence identity to SEQ ID NO: 38) can be cloned or amplified by in vitro methods, such as the polymerase chain reaction (PCR), the ligase chain reaction (LCR), the transcription-based amplification system (TAS), the self-sustained sequence replication system (3SR) and the QP replicase amplification system (QB). In addition, nucleic acids encoding a Nmb protein (such as a nucleic acid molecule having least 50%, at least 60%, at least 70%, at least 75%, at least 80%, at least 90%, at least 95%, at least 96%, at least 97%, at least 98%, at least 99% or 100% sequence identity to SEQ ID NO: 2, or encoding a protein having least 50%, at least 60%, at least 70%, at least 75%, at least 80%, at least 90%, at least 95%, at least 96%, at least 97%, at least 98%, at least 99% or 100% sequence identity to SEQ ID NO: 1, 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37) or Nme protein (such as one encoding a protein having at least 50%, at least 60%, at least 70%, at least 80%, at least 90%, at least 95%, at least 96%, at least 97%, at least 98%, at least 99%, or 100% sequence identity to SEQ ID NO: 38) can be prepared by cloning techniques (such as those found in Sambrook et al. (ed.), Molecular Cloning: A Laboratory Manual 2nd ed., vol. 1-3, Cold Spring Harbor Laboratory Press, Cold Spring, Harbor, N.Y., 1989, and Ausubel et al., (1987) in “Current Protocols in Molecular Biology,” John Wiley and Sons, New York, N.Y.).

Nucleic acid sequences encoding an Nmb protein (such as a nucleic acid molecule having at least 50%, at least 60%, at least 70%, at least 75%, at least 80%, at least 90%, at least 95%, at least 96%, at least 97%, at least 98%, at least 99% or 100% sequence identity to SEQ ID NO: 2, or encoding a protein having at least 50%, at least 60%, at least 70%, at least 75%, at least 80%, at least 90%, at least 95%, at least 96%, at least 97%, at least 98%, at least 99% or 100% sequence identity to SEQ ID NO: 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37) or Nme protein (such as one encoding a protein having at least 50%, at least 60%, at least 70%, at least 80%, at least 90%, at least 95%, at least 96%, at least 97%, at least 98%, at least 99%, or 100% sequence identity to SEQ ID NO: 38) can be prepared by any suitable method including, for example, cloning of appropriate sequences or by direct chemical synthesis by methods such as the phosphotriester method of Narang et al., Meth. Enzymol. 68:90-99, 1979; the phosphodiester method of Brown et al., Meth. Enzymol. 68:109-151, 1979; the diethylphosphoramidite method of Beaucage et al., Tetra. Lett. 22:1859-1862, 1981; the solid phase phosphoramidite triester method described by Beaucage & Caruthers, Tetra. Letts. 22(20):1859-1862, 1981, for example, using an automated synthesizer as described in, for example, Needham-VanDevanter et al., Nucl. Acids Res. 12:6159-6168, 1984; and, the solid support method of U.S. Pat. No. 4,458,066. Chemical synthesis produces a single stranded oligonucleotide. This can be converted into double stranded DNA by hybridization with a complementary sequence, or by polymerization with a DNA polymerase using the single strand as a template.

In one example, an Nmb protein (such as one having at least 50%, at least 60%, at least 70%, at least 75%, at least 80%, at least 90%, at least 95%, at least 96%, at least 97%, at least 98%, at least 99% or 100% sequence identity to SEQ ID NO: 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37) or Nme protein (such as having at least 50%, at least 60%, at least 70%, at least 80%, at least 90%, at least 95%, at least 96%, at least 97%, at least 98%, at least 99%, or 100% sequence identity to SEQ ID NO: 38) is prepared by inserting a cDNA which encodes a Nmb protein (such as a nucleic acid molecule having at least 50%, at least 60%, at least 70%, at least 75%, at least 80%, at least 90%, at least 95%, at least 96%, at least 97%, at least 98%, at least 99% or 100% sequence identity to SEQ ID NO: 2) or Nme protein into a vector. The insertion can be made so that the Nmb or Nme protein is read in frame so that the Nmb or Nme protein is produced.

The Nmb nucleic acid coding sequence (such as a nucleic acid molecule having at least 50%, at least 60%, at least 70%, at least 80%, at least 90%, at least 95%, at least 96%, at least 97%, at least 98%, at least 99% or 100% sequence identity to SEQ ID NO: 2, or encoding a protein having at least 50%, at least 60%, at least 70%, at least 80%, at least 90%, at least 95%, at least 96%, at least 97%, at least 98%, at least 99% or 100% sequence identity to SEQ ID NO: 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37) or Nme nucleic acid coding sequence (such as one encoding a protein having at least 50%, at least 60%, at least 70%, at least 80%, at least 90%, at least 95%, at least 96%, at least 97%, at least 98%, at least 99%, or 100% sequence identity to SEQ ID NO: 38) can be inserted into an expression vector including, but not limited to a plasmid, virus or other vehicle that can be manipulated to allow insertion or incorporation of sequences and can be expressed in either prokaryotes or eukaryotes. Hosts can include microbial, yeast, insect, plant and mammalian organisms. The vector can encode a selectable marker, such as a thymidine kinase gene, antibiotic resistance gene, or fluorescent protein.

Nucleic acid sequences encoding an Nmb protein (such as a nucleic acid molecule having at least 50%, at least 60%, at least 70%, at least 80%, at least 90%, at least 95%, at least 96%, at least 97%, at least 98%, at least 99% or 100% sequence identity to SEQ ID NO: 2, or encoding a protein having at least 50%, at least 60%, at least 70%, at least 80%, at least 90%, at least 95%, at least 96%, at least 97%, at least 98%, at least 99% or 100% sequence identity to SEQ ID NO: 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37) or Nme protein (such as one encoding a protein having at least 50%, at least 60%, at least 70%, at least 80%, at least 90%, at least 95%, at least 96%, at least 97%, at least 98%, at least 99%, or 100% sequence identity to SEQ ID NO: 38) can be operatively linked to expression control sequences. An expression control sequence operatively linked to a Nmb or Nme protein coding sequence is ligated such that expression of the Nmb or Nme coding sequence is achieved under conditions compatible with the expression control sequences. Exemplary expression control sequences include, but are not limited to promoters, enhancers, transcription terminators, a start codon (i.e., ATG) in front of a Nmb or Nme protein-encoding gene, splicing signal for introns, maintenance of the correct reading frame of that gene to permit proper translation of mRNA, and stop codons.

In one embodiment, vectors are used for expression in yeast such as S. cerevisiae, P. pastoris, or Kluyveromyces lactis. Exemplary promoters for use in yeast expression systems include the constitutive promoters, plasma membrane H⁺-ATPase (PMA1), glyceraldehyde-3-phosphate dehydrogenase (GPD), phosphoglycerate kinase-1 (PGK1), alcohol dehydrogenase-1 (ADH1), and pleiotropic drug-resistant pump (PDR5). In addition, inducible promoters are of use, such as GALI-10 (induced by galactose), PHO5 (induced by low extracellular inorganic phosphate), and tandem heat shock HSE elements (induced by temperature elevation to 37° C.). Promoters that direct variable expression in response to a titratable inducer include the methionine-responsive MET3 and MET25 promoters and copper-dependent CUP1 promoters. Any of these promoters may be cloned into multicopy (2μ) or single copy (CEN) plasmids to give an additional level of control in expression level. The plasmids can include nutritional markers (such as URA3, ADE3, HIS1, and others) for selection in yeast and antibiotic resistance (AMP) for propagation in bacteria. Plasmids for expression on K. lactis are known, such as pKLAC1. Thus, in one example, after amplification in bacteria, plasmids can be introduced into the corresponding yeast auxotrophs by methods similar to bacterial transformation. The nucleic acid molecules encoding a Nmb protein (such as one having at least 50%, at least 60%, at least 70%, at least 80%, at least 90%, at least 95%, at least 96%, at least 97%, at least 98%, at least 99% or 100% sequence identity to SEQ ID NO: 2, or encoding a protein having at least 50%, at least 60%, at least 70%, at least 80%, at least 90%, at least 95%, at least 96%, at least 97%, at least 98%, at least 99% or 100% sequence identity to SEQ ID NO: 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37) or Nme protein (such as one encoding a protein having at least 50%, at least 60%, at least 70%, at least 80%, at least 90%, at least 95%, at least 96%, at least 97%, at least 98%, at least 99%, or 100% sequence identity to SEQ ID NO: 38) can also be designed to express in insect cells.

A Nmb protein (such as one having at least 50%, at least 60%, at least 70%, at least 80%, at least 90%, at least 95%, at least 96%, at least 97%, at least 98%, at least 99% or 100% sequence identity to SEQ ID NO: 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37) or Nme protein (such as one having least 50%, at least 60%, at least 70%, at least 80%, at least 90%, at least 95%, at least 96%, at least 97%, at least 98%, at least 99%, or 100% sequence identity to SEQ ID NO: 38) can be expressed in a yeast strain. For example, seven pleiotropic drug-resistant transporters, YOR1, SNQ2, PDR5, YCF1, PDR10, PDR11, and PDR15, together with their activating transcription factors, PDR1 and PDR3, have been simultaneously deleted in yeast host cells, rendering the resultant strain sensitive to drugs. Yeast strains with altered lipid composition of the plasma membrane, such as the erg6 mutant defective in ergosterol biosynthesis, can also be utilized. Proteins that are highly sensitive to proteolysis can be expressed in a yeast cell lacking the master vacuolar endopeptidase Pep4, which controls the activation of other vacuolar hydrolases. Heterologous expression in strains carrying temperature-sensitive (ts) alleles of genes can be employed if the corresponding null mutant is inviable.

Viral vectors can also be prepared that encode a Nmb protein (such as those that include a nucleic acid molecule having at least 50%, at least 60%, at least 70%, at least 80%, at least 90%, at least 95%, at least 96%, at least 97%, at least 98%, at least 99% or 100% sequence identity to SEQ ID NO: 2, or a nucleic acid molecule that encodes a protein having at least 50%, at least 60%, at least 70%, at least 80%, at least 90%, at least 95%, at least 96%, at least 97%, at least 98%, at least 99% or 100% sequence identity to SEQ ID NO: 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37) or Nme protein (such as one encoding a protein having at least 50%, at least 60%, at least 70%, at least 80%, at least 90%, at least 95%, at least 96%, at least 97%, at least 98%, at least 99%, or 100% sequence identity to SEQ ID NO: 38). Exemplary viral vectors include polyoma, SV40, adenovirus, vaccinia virus, adeno-associated virus (AAV), herpes viruses including HSV and EBV, Sindbis viruses, alphaviruses and retroviruses of avian, murine, and human origin. Baculovirus (Autographa californica multinuclear polyhedrosis virus; AcMNPV) vectors can also be used. Other suitable vectors include retrovirus vectors, orthopox vectors, avipox vectors, fowlpox vectors, capripox vectors, suipox vectors, adenoviral vectors, herpes virus vectors, alpha virus vectors, baculovirus vectors, Sindbis virus vectors, vaccinia virus vectors and poliovirus vectors. Specific exemplary vectors are poxvirus vectors such as vaccinia virus, fowlpox virus and a highly attenuated vaccinia virus (MVA), adenovirus, baculovirus and the like. Pox viruses of use include orthopox, suipox, avipox, and capripox virus. Orthopox include vaccinia, ectromelia, and raccoon pox. One example of an orthopox of use is vaccinia. Avipox includes fowlpox, canary pox and pigeon pox. Capripox include goatpox and sheeppox. In one example, the suipox is swinepox. Other viral vectors that can be used include other DNA viruses such as herpes virus and adenoviruses, and RNA viruses such as retroviruses and polio.

Viral vectors that encode a Nmb protein (such as those that include a nucleic acid molecule having at least 50%, at least 60%, at least 70%, at least 80%, at least 90%, at least 95%, at least 96%, at least 97%, at least 98%, at least 99% or 100% sequence identity to SEQ ID NO: 2, or a nucleic acid molecule that encodes a protein having at least 50%, at least 60%, at least 70%, at least 80%, at least 90%, at least 95%, at least 96%, at least 97%, at least 98%, at least 99% or 100% sequence identity to SEQ ID NO: 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37) or a Nme protein (such as a nucleic acid molecule that encodes a protein having at least 50%, at least 60%, at least 70%, at least 80%, at least 90%, at least 95%, at least 96%, at least 97%, at least 98%, at least 99% or 100% sequence identity to SEQ ID NO: 38) can include at least one expression control element operationally linked to the nucleic acid sequence encoding the Nmb protein or Nme protein. The expression control elements are inserted in the vector to control and regulate the expression of the nucleic acid sequence. Examples of expression control elements of use in these vectors includes, but is not limited to, lac system, operator and promoter regions of phage lambda, yeast promoters and promoters derived from polyoma, adenovirus, retrovirus or SV40. Additional operational elements include, but are not limited to, leader sequence, termination codons, polyadenylation signals and any other sequences necessary for the appropriate transcription and subsequent translation of the nucleic acid sequence encoding the Nmb protein in the host system. The expression vector can contain additional elements necessary for the transfer and subsequent replication of the expression vector containing the nucleic acid sequence in the host system. Examples of such elements include, but are not limited to, origins of replication and selectable markers. Such vectors can be constructed using conventional methods (Ausubel et al., (1987) in “Current Protocols in Molecular Biology,” John Wiley and Sons, New York, N.Y.) and are commercially available.

In one example, the viral vector that encodes a Nmb protein (such as those that include a nucleic acid molecule having at least 50%, at least 60%, at least 70%, at least 80%, at least 90%, at least 95%, at least 96%, at least 97%, at least 98%, at least 99% or 100% sequence identity to SEQ ID NO: 2, or a nucleic acid molecule that encodes a protein having at least 50%, at least 60%, at least 70%, at least 80%, at least 90%, at least 95%, at least 96%, at least 97%, at least 98%, at least 99% or 100% sequence identity to SEQ ID NO: 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37) or a Nme protein (such as a nucleic acid molecule that encodes a protein having at least 50%, at least 60%, at least 70%, at least 80%, at least 90%, at least 95%, at least 96%, at least 97%, at least 98%, at least 99% or 100% sequence identity to SEQ ID NO: 38) is a lentiviral or AAV vector, and includes a promoter operably linked to the Nmb coding sequence. In some examples, the promoter is a constitutive promoter, such as CMV, beta actin, or T7, or a tissue specific promoter, such as a lung-specific promoter (e.g., lung-specific surfactant protein B gene promoter, SP-B promoter, or CC10 promoter) or skin-specific promoter (e.g., kerain 14 promoter, filaggrin promoter, or transglutaminase 3 promoter).

Methods for preparing recombinant virus containing a heterologous DNA sequence encoding the Nmb protein (such as those that include a nucleic acid molecule having at least 50%, at least 60%, at least 70%, at least 80%, at least 90%, at least 95%, at least 96%, at least 97%, at least 98%, at least 99% or 100% sequence identity to SEQ ID NO: 2, or a nucleic acid molecules that encodes a protein having at least 50%, at least 60%, at least 70%, at least 80%, at least 90%, at least 95%, at least 96%, at least 97%, at least 98%, at least 99% or 100% sequence identity to SEQ ID NO: 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37) or Nme protein (such as a nucleic acid molecule that encodes a protein having at least 50%, at least 60%, at least 70%, at least 80%, at least 90%, at least 95%, at least 96%, at least 97%, at least 98%, at least 99% or 100% sequence identity to SEQ ID NO: 38) are known. Such techniques involve, for example, homologous recombination between the viral sequences flanking the Nmb or Nme coding sequence in a donor plasmid and homologous sequences present in the parental virus. The vector can be constructed for example by using a unique restriction endonuclease site that is naturally present or artificially inserted in the parental viral vector to insert the heterologous DNA.

When the cell into which the Nmb or Nme coding sequence is introduced is eukaryotic, transfection methods include calcium phosphate coprecipitates, mechanical procedures such as microinjection, electroporation, insertion of a plasmid encased in liposomes, or viral vectors. Eukaryotic cells can also be co-transformed with polynucleotide sequences encoding a Nmb protein (such as those that include a nucleic acid molecule having at least 50%, at least 60%, at least 70%, at least 80%, at least 90%, at least 95%, at least 96%, at least 97%, at least 98%, at least 99% or 100% sequence identity to SEQ ID NO: 2, or a nucleic acid molecule that encodes a protein having at least 50%, at least 60%, at least 70%, at least 80%, at least 90%, at least 95%, at least 96%, at least 97%, at least 98%, at least 99% or 100% sequence identity to SEQ ID NO: 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37) or a Nme protein (such as a nucleic acid molecule that encodes a protein having at least 50%, at least 60%, at least 70%, at least 80%, at least 90%, at least 95%, at least 96%, at least 97%, at least 98%, at least 99% or 100% sequence identity to SEQ ID NO: 38), and a second foreign DNA molecule encoding a selectable phenotype, such as the herpes simplex thymidine kinase gene. Another method is to use a eukaryotic viral vector, such as simian virus 40 (SV40) or bovine papilloma virus, to transiently infect or transform eukaryotic cells and express the protein (see for example, Eukaryotic Viral Vectors, Cold Spring Harbor Laboratory, Gluzman ed., 1982). Expression systems such as plasmids and vectors can be used to produce Nmb proteins in cells including higher eukaryotic cells such as the COS, CHO, HeLa and myeloma cell lines.

### E. Administration and Dosing

Pharmaceutical compositions that include a Nmb protein (such as a protein having at least 50%, at least 60%, at least 70%, at least 80%, at least 90%, at least 95%, at least 96%, at least 97%, at least 98%, at least 99% or 100% sequence identity to SEQ ID NO: 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37) or a Nme protein (such as a protein having at least 50%, at least 60%, at least 70%, at least 80%, at least 90%, at least 95%, at least 96%, at least 97%, at least 98%, at least 99% or 100% sequence identity to SEQ ID NO: 38) or a nucleic acid encoding a Nmb protein (such as a nucleic acid molecule at least 50%, at least 60%, at least 70%, at least 80%, having at least 50%, at least 60%, at least 70%, at least 80%, at least 90%, at least 95%, at least 96%, at least 97%, at least 98%, at least 99% or 100% sequence identity to SEQ ID NO: 2, or a nucleic acid molecule that encodes a protein having at least 50%, at least 60%, at least 70%, at least 80%, at least 90%, at least 95%, at least 96%, at least 97%, at least 98%, at least 99% or 100% sequence identity to SEQ ID NO: 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37) or a Nme protein (such as a nucleic acid molecule that encodes a protein having at least 50%, at least 60%, at least 70%, at least 80%, at least 90%, at least 95%, at least 96%, at least 97%, at least 98%, at least 99% or 100% sequence identity to SEQ ID NO: 38), can be formulated with an appropriate pharmaceutically acceptable carrier (such as water or saline), depending upon the particular mode of administration chosen. Such compositions can be administered to a subject with a neurological disorder using the disclosed methods. In one example, the pharmaceutical composition is suitable for injection, such as injection into the skin, vein, or muscle.

In some embodiments, the pharmaceutical composition consists essentially of a Nmb protein (such as a protein having at least 50%, at least 60%, at least 70%, at least 80%, at least 90%, at least 95%, at least 96%, at least 97%, at least 98%, at least 99% or 100% sequence identity to SEQ ID NO: 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37), a Nme protein (such as a protein having at least 50%, at least 60%, at least 70%, at least 80%, at least 90%, at least 95%, at least 96%, at least 97%, at least 98%, at least 99% or 100% sequence identity to SEQ ID NO: 38), or a nucleic acid encoding a Nmb protein (such as a nucleic acid molecule having at least 50%, at least 60%, at least 70%, at least 80%, at least 90%, at least 95%, at least 96%, at least 97%, at least 98%, at least 99% or 100% sequence identity to SEQ ID NO: 2, or a nucleic acid molecule that encodes a protein having at least 50%, at least 60%, at least 70%, at least 80%, at least 90%, at least 95%, at least 96%, at least 97%, at least 98%, at least 99% or 100% sequence identity to SEQ ID NO: 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37), or a Nme protein (such as a nucleic acid molecule that encodes a protein having at least 50%, at least 60%, at least 70%, at least 80%, at least 90%, at least 95%, at least 96%, at least 97%, at least 98%, at least 99% or 100% sequence identity to SEQ ID NO: 38), and a pharmaceutically acceptable carrier. In these embodiments, additional therapeutically effective agents are not included in the compositions.

In other embodiments, the pharmaceutical composition includes a Nmb protein such as a protein having at least 50%, at least 60%, at least 70%, at least 80%, at least 90%, at least 95%, at least 96%, at least 97%, at least 98%, at least 99% or 100% sequence identity to SEQ ID NO: 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37), a Nme protein (such as a protein having at least 50%, at least 60%, at least 70%, at least 80%, at least 90%, at least 95%, at least 96%, at least 97%, at least 98%, at least 99% or 100% sequence identity to SEQ ID NO: 38), or a nucleic acid encoding a Nmb protein (such as a nucleic acid molecule having at least 50%, at least 60%, at least 70%, at least 80%, at least 90%, at least 95%, at least 96%, at least 97%, at least 98%, at least 99% or 100% sequence identity to SEQ ID NO: 2, or a nucleic acid molecule that encodes a protein having at least 50%, at least 60%, at least 70%, at least 80%, at least 90%, at least 95%, at least 96%, at least 97%, at least 98%, at least 99% or 100% sequence identity to SEQ ID NO: 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37), or a Nme protein (such as a nucleic acid molecule that encodes a protein having at least 50%, at least 60%, at least 70%, at least 80%, at least 90%, at least 95%, at least 96%, at least 97%, at least 98%, at least 99% or 100% sequence identity to SEQ ID NO: 38), and a pharmaceutically acceptable carrier. Additional therapeutic agents, such as agents for the treatment of an inflammatory disorder caused by a type 2 cytokine (such as a disorder shown in Table 1), can be included. Thus, the pharmaceutical compositions can include a therapeutically effective amount of another agent. Examples of such agents include, without limitation, PGE2, those listed in section “F” below and in Table 1, or combinations thereof.

The pharmaceutically acceptable carriers and excipients useful in this disclosure are conventional. See, e.g., Remington: The Science and Practice of Pharmacy, The University of the Sciences in Philadelphia, Editor, Lippincott, Williams, & Wilkins, Philadelphia, PA, 22^{st} Edition (2013). For instance, parenteral formulations usually include injectable fluids that are pharmaceutically and physiologically acceptable fluid vehicles such as water, physiological saline, other balanced salt solutions, aqueous dextrose, glycerol or the like. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, pH buffering agents, or the like, for example sodium acetate or sorbitan monolaurate. Excipients that can be included are, for instance, other proteins, such as human serum albumin or plasma preparations.

In some embodiments, a Nmb protein (such as a protein having at least 50%, at least 60%, at least 70%, at least 80%, at least 90%, at least 95%, at least 96%, at least 97%, at least 98%, at least 99% or 100% sequence identity to SEQ ID NO: 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37), a Nme protein (such as a protein having at least 50%, at least 60%, at least 70%, at least 80%, at least 90%, at least 95%, at least 96%, at least 97%, at least 98%, at least 99% or 100% sequence identity to SEQ ID NO: 38), or a nucleic acid encoding a Nmb protein (such as a nucleic acid molecule having at least 50%, at least 60%, at least 70%, at least 80%, at least 90%, at least 95%, at least 96%, at least 97%, at least 98%, at least 99% or 100% sequence identity to SEQ ID NO: 2, or a nucleic acid molecule that encodes a protein having at least 50%, at least 60%, at least 70%, at least 80%, at least 90%, at least 95%, at least 96%, at least 97%, at least 98%, at least 99% or 100% sequence identity to SEQ ID NO: 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37), or a Nme protein (such as a nucleic acid molecule that encodes a protein having at least 50%, at least 60%, at least 70%, at least 80%, at least 90%, at least 95%, at least 96%, at least 97%, at least 98%, at least 99% or 100% sequence identity to SEQ ID NO: 38), is included in a controlled release formulation, for example, a microencapsulated formulation. Various types of biodegradable and biocompatible polymers, methods can be used, and methods of encapsulating a variety of synthetic compounds, proteins and nucleic acids can be used (see, for example, U.S. Patent Publication Nos. 2007/0148074; 2007/0092575; and 2006/0246139; U.S. Pat. Nos. 4,522,811; 5,753,234; and 7,081,489; PCT Publication No. WO/2006/052285; Benita, Microencapsulation: Methods and Industrial Applications, 2^{nd} ed., CRC Press, 2006).

In other embodiments, a Nmb protein (such as a protein having at least 50%, at least 60%, at least 70%, at least 80%, at least 90%, at least 95%, at least 96%, at least 97%, at least 98%, at least 99% or 100% sequence identity to SEQ ID NO: 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37), or a Nme protein (such as a protein having at least 50%, at least 60%, at least 70%, at least 80%, at least 90%, at least 95%, at least 96%, at least 97%, at least 98%, at least 99% or 100% sequence identity to SEQ ID NO: 38), or a nucleic acid encoding a Nmb protein (such as a nucleic acid molecule having at least 50%, at least 60%, at least 70%, at least 80%, at least 90%, at least 95%, at least 96%, at least 97%, at least 98%, at least 99% or 100% sequence identity to SEQ ID NO: 2 or a nucleic acid molecule that encodes a protein having at least 50%, at least 60%, at least 70%, at least 80%, at least 90%, at least 95%, at least 96%, at least 97%, at least 98%, at least 99% or 100% sequence identity to SEQ ID NO: 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37) or a Nme protein (such as a nucleic acid molecule that encodes a protein having at least 50%, at least 60%, at least 70%, at least 80%, at least 90%, at least 95%, at least 96%, at least 97%, at least 98%, at least 99% or 100% sequence identity to SEQ ID NO: 38), is included in a nanodispersion system. See, e.g., U.S. Pat. No. 6,780,324; U.S. Pat. Publication No. 2009/0175953. For example, a nanodispersion system includes a biologically active agent and a dispersing agent (such as a polymer, copolymer, or low molecular weight surfactant). Exemplary polymers or copolymers that can be used include polyvinylpyrrolidone (PVP), poly(D,L-lactic acid) (PLA), poly(D,L-lactic-co-glycolic acid (PLGA), poly(ethylene glycol). Exemplary low molecular weight surfactants include sodium dodecyl sulfate, hexadecyl pyridinium chloride, polysorbates, sorbitans, poly(oxyethylene) alkyl ethers, poly(oxyethylene) alkyl esters, and combinations thereof. In one example, the nanodispersion system includes PVP and ODP or a variant thereof (such as 80/20 w/w). In some examples, the nanodispersion is prepared using the solvent evaporation method, see for example, Kanaze et al., Drug Dev. Indus. Pharm. 36:292-301, 2010; Kanaze et al., J. Appl. Polymer Sci. 102:460-471, 2006. With regard to the administration of nucleic acids, one approach to administration of nucleic acids is direct treatment with a viral vector, such as a lentiviral or AAV vector. As described above, the nucleotide sequence encoding a Nmb protein (such as a protein having at least 50%, at least 60%, at least 70%, at least 80%, at least 90%, at least 95%, at least 96%, at least 97%, at least 98%, at least 99% or 100% sequence identity to SEQ ID NO: 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37), such as a nucleic acid molecule having at least 50%, at least 60%, at least 70%, at least 80%, at least 90%, at least 95%, at least 96%, at least 97%, at least 98%, at least 99% or 100% sequence identity to SEQ ID NO: 2, or a nucleic acid molecule that encodes a protein having at least 50%, at least 60%, at least 70%, at least 80%, at least 90%, at least 95%, at least 96%, at least 97%, at least 98%, at least 99% or 100% sequence identity to SEQ ID NO: 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37) or a nucleic acid molecule that encodes a Nme protein (such as a nucleic acid molecule that encodes a protein having at least 50%, at least 60%, at least 70%, at least 80%, at least 90%, at least 95%, at least 96%, at least 97%, at least 98%, at least 99% or 100% sequence identity to SEQ ID NO: 38) can be placed under the control of a promoter to increase expression of the Nmb or Nme protein.

The Nmb protein (such as a protein having at least 50%, at least 60%, at least 70%, at least 80%, at least 90%, at least 95%, at least 96%, at least 97%, at least 98%, at least 99% or 100% sequence identity to SEQ ID NO: 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37) or Nme protein (such as a protein having at least 50%, at least 60%, at least 70%, at least 80%, at least 90%, at least 95%, at least 96%, at least 97%, at least 98%, at least 99% or 100% sequence identity to SEQ ID NO: 38), or a Nmb coding sequence (such as a nucleic acid molecule having at least 50%, at least 60%, at least 70%, at least 80%, at least 90%, at least 95%, at least 96%, at least 97%, at least 98%, at least 99% or 100% sequence identity to SEQ ID NO: 2) or a Nme coding sequence (such as a nucleic acid molecule that encodes a protein having at least 50%, at least 60%, at least 70%, at least 80%, at least 90%, at least 95%, at least 96%, at least 97%, at least 98%, at least 99% or 100% sequence identity to SEQ ID NO: 38) can be administered alone, or in various combinations, and in combination with other therapeutic compositions. In addition, the Nmb protein (such as a protein having at least 50%, at least 60%, at least 70%, at least 80%, at least 90%, at least 95%, at least 96%, at least 97%, at least 98%, at least 99% or 100% sequence identity to SEQ ID NO: 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37) or Nme protein (such as a protein having at least 50%, at least 60%, at least 70%, at least 80%, at least 90%, at least 95%, at least 96%, at least 97%, at least 98%, at least 99% or 100% sequence identity to SEQ ID NO: 38), or a Nmb coding sequence (such as a nucleic acid molecule having at least 50%, at least 60%, at least 70%, at least 80%, at least 90%, at least 95%, at least 96%, at least 97%, at least 98%, at least 99% or 100% sequence identity to SEQ ID NO: 2) or Nme coding sequence (such as a nucleic acid molecule that encodes a protein having at least 50%, at least 60%, at least 70%, at least 80%, at least 90%, at least 95%, at least 96%, at least 97%, at least 98%, at least 99% or 100% sequence identity to SEQ ID NO: 38) can be administered systemically or locally.

Many types of release delivery systems can be used. Examples include polymer based systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems, such as lipids including sterols such as cholesterol, cholesterol esters and fatty acids or neutral fats such as mono- di- and tri-glycerides; hydrogel release systems; silastic systems; peptide based systems; wax coatings; compressed tablets using conventional binders and excipients; partially fused implants; and the like. Specific examples include, but are not limited to: (a) erosional systems in which Nmb protein (such as a protein having at least 50%, at least 60%, at least 70%, at least 80%, at least 90%, at least 95%, at least 96%, at least 97%, at least 98%, at least 99% or 100% sequence identity to SEQ ID NO: 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37) or Nme protein (such as a protein having at least 50%, at least 60%, at least 70%, at least 80%, at least 90%, at least 95%, at least 96%, at least 97%, at least 98%, at least 99% or 100% sequence identity to SEQ ID NO: 38), or a nucleic acid encoding a Nmb protein (such as a nucleic acid molecule having at least 50%, at least 60%, at least 70%, at least 80%, at least 90%, at least 95%, at least 96%, at least 97%, at least 98%, at least 99% or 100% sequence identity to SEQ ID NO: 2, or a nucleic acid molecule that encodes a protein having at least 50%, at least 60%, at least 70%, at least 80%, at least 90%, at least 95%, at least 96%, at least 97%, at least 98%, at least 99% or 100% sequence identity to SEQ ID NO: 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37) or a Nme coding sequence (such as a nucleic acid molecule that encodes a protein having at least 50%, at least 60%, at least 70%, at least 80%, at least 90%, at least 95%, at least 96%, at least 97%, at least 98%, at least 99% or 100% sequence identity to SEQ ID NO: 38), is contained in a form within a matrix such as those described in U.S. Pat. Nos. 4,452,775; 4,667,014; 4,748,034; 5,239,660; and 6,218,371 and (b) diffusional systems in which an active component permeates at a controlled rate from a polymer such as described in U.S. Pat. Nos. 3,832,253 and 3,854,480. In addition, pump-based hardware delivery systems can be used, some of which are adapted for implantation.

A long-term sustained release implant can be suitable for treatment of chronic conditions, such as inflammatory disorders (e.g., see Table 1). Long-term release, as used herein, means that the implant is constructed and arranged to deliver therapeutic levels of the active ingredient for at least 30 days, or at least 60 days. Long-term sustained release implants include the release systems described above. These systems have been described for use with nucleic acids (see U.S. Pat. No. 6,218,371). For use in vivo, nucleic acids and peptides are relatively resistant to degradation (such as via endo- and exo-nucleases). Thus, modifications of a Nmb or Nme protein, such as the inclusion of a C-terminal amide, can be used.

In some examples, the Nmb protein (such as a protein having at least 50%, at least 60%, at least 70%, at least 80%, at least 90%, at least 95%, at least 96%, at least 97%, at least 98%, at least 99% or 100% sequence identity to SEQ ID NO: 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37) or Nme protein (such as a protein having at least 50%, at least 60%, at least 70%, at least 80%, at least 90%, at least 95%, at least 96%, at least 97%, at least 98%, at least 99% or 100% sequence identity to SEQ ID NO: 38), or a nucleic acid encoding a Nmb protein (such as a nucleic acid molecule having at least 50%, at least 60%, at least 70%, at least 80%, at least 90%, at least 95%, at least 96%, at least 97%, at least 98%, at least 99% or 100% sequence identity to SEQ ID NO: 2, or a nucleic acid molecule that encodes a protein having at least 50%, at least 60%, at least 70%, at least 80%, at least 90%, at least 95%, at least 96%, at least 97%, at least 98%, at least 99% or 100% sequence identity to SEQ ID NO: 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37) or a Nme coding sequence (such as a nucleic acid molecule that encodes a protein having at least 50%, at least 60%, at least 70%, at least 80%, at least 90%, at least 95%, at least 96%, at least 97%, at least 98%, at least 99% or 100% sequence identity to SEQ ID NO: 38), is administered locally to the airway, for example in the form of an aerosol spray (which can include solid or liquid particles), such as from pressurized packs or a nebulizer, with the use of a propellant, such as dichlorodifluoromethane, trichlorofluoromethane, dichlorotetrafluoroethane, carbon dioxide or other suitable gas. In the case of a pressurized aerosol, the dosage unit can be determined by providing a valve to deliver a metered amount. Capsules and cartridges for use in an inhaler or insufflator can be formulated containing a powder mix of the compound and a suitable powder base such as lactose or starch.

In some embodiments, the Nmb protein (such as a protein having at least 50%, at least 60%, at least 70%, at least 80%, at least 90%, at least 95%, at least 96%, at least 97%, at least 98%, at least 99% or 100% sequence identity to SEQ ID NO: 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37) or Nme protein (such as a protein having at least 50%, at least 60%, at least 70%, at least 80%, at least 90%, at least 95%, at least 96%, at least 97%, at least 98%, at least 99% or 100% sequence identity to SEQ ID NO: 38), or a nucleic acid encoding a Nmb protein (such as a nucleic acid molecule having at least 50%, at least 60%, at least 70%, at least 80%, at least 90%, at least 95%, at least 96%, at least 97%, at least 98%, at least 99% or 100% sequence identity to SEQ ID NO: 2, or a nucleic acid molecule that encodes a protein having at least 50%, at least 60%, at least 70%, at least 80%, at least 90%, at least 95%, at least 96%, at least 97%, at least 98%, at least 99% or 100% sequence identity to SEQ ID NO: 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37) or a Nme coding sequence (such as a nucleic acid molecule that encodes a protein having at least 50%, at least 60%, at least 70%, at least 80%, at least 90%, at least 95%, at least 96%, at least 97%, at least 98%, at least 99% or 100% sequence identity to SEQ ID NO: 38) is administered by inhalation. For example, the Nmb protein (such as a protein having at least 50%, at least 60%, at least 70%, at least 80%, at least 90%, at least 95%, at least 96%, at least 97%, at least 98%, at least 99% or 100% sequence identity to SEQ ID NO: 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37) or Nme protein (such as a protein having at least 50%, at least 60%, at least 70%, at least 80%, at least 90%, at least 95%, at least 96%, at least 97%, at least 98%, at least 99% or 100% sequence identity to SEQ ID NO: 38), or a nucleic acid encoding a Nmb protein (such as a nucleic acid molecule having at least 50%, at least 60%, at least 70%, at least 80%, at least 90%, at least 95%, at least 96%, at least 97%, at least 98%, at least 99% or 100% sequence identity to SEQ ID NO: 2, or a nucleic acid molecule that encodes a protein having at least 50%, at least 60%, at least 70%, at least 80%, at least 90%, at least 95%, at least 96%, at least 97%, at least 98%, at least 99% or 100% sequence identity to SEQ ID NO: 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37) or a Nme coding sequence (such as a nucleic acid molecule that encodes a protein having at least 50%, at least 60%, at least 70%, at least 80%, at least 90%, at least 95%, at least 96%, at least 97%, at least 98%, at least 99% or 100% sequence identity to SEQ ID NO: 38) is administered in an aerosolized form, such as using a nebulizer, a metered dose inhaler (MDI) or a dry powder inhaler (DPI). Technologies of use include micropump nebulizers (such as the AEROGENGO® system), jet nebulizers designed to produce large fine particle fractions (such as the PARI LC STAR®), jet nebulizers developing less shear during atomization (such as the HUDSON MICROMIST®), and ultrasonic nebulizers (such as the DeVilbiss ULTRA-NEB®).

Formulations suitable for use with a nebulizer, either jet or ultrasonic, can include an Nmb protein or nucleic acid molecule dissolved in water at a concentration of about 0.1 to 25 mg per mL of solution. The formulation can also include a buffer and a simple sugar (such as for protein stabilization and regulation of osmotic pressure). The nebulizer formulation can also contain a surfactant, to reduce or prevent surface induced aggregation of the Nmb or Nme protein or nucleic acid molecule caused by atomization of the solution in forming the aerosol (U.S. Patent Application Publication No. 2007/0065367).

Formulations for use with a MDI device generally includes a finely divided powder containing the Nmb or Nme protein or nucleic acid molecule suspended in a propellant with the aid of a surfactant. The propellant can be any conventional material employed for this purpose, such as a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant (U.S. Patent Application Publication No. 2007/0065367). The device of the inhalation system can deliver a single dose (such as by a blister pack), or it can be multi-dose in design. To ensure accuracy of dosing, the delivery of the formulation can be programmed via a microprocessor to occur at a certain point in the inhalation cycle. In some cases, the MDI is portable and hand held.

A dry powder inhalator (DPI) also can be used as the aerosol delivery device. The basic design of a DPI includes a metering system, a powdered composition and a method to disperse the composition. Forces like rotation and vibration can be used to disperse the composition. The metering and dispersion systems can be mechanically or electrically driven and can be microprocessor-programmable. The device can be portable and hand held. The inhalator can be multi- or single-dose in design and use such options as hard gelatin capsules or blister packages for accurate unit doses. The Nmb or Nme protein or nucleic acid molecule can be dispersed from the device by passive inhalation (such as the patient's own inspiratory effort), or an active dispersion system can be employed. The dry powder of the therapeutic composition can be sized via processes such as jet milling, spray dying and supercritical fluid manufacture. Acceptable excipients such as the sugars mannitol and maltose can be used in the preparation of the powdered formulations.

The Nmb protein (such as a protein having at least 50%, at least 60%, at least 70%, at least 80%, at least 90%, at least 95%, at least 96%, at least 97%, at least 98%, at least 99% or 100% sequence identity to SEQ ID NO: 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37) or Nme protein (such as a protein having at least 50%, at least 60%, at least 70%, at least 80%, at least 90%, at least 95%, at least 96%, at least 97%, at least 98%, at least 99% or 100% sequence identity to SEQ ID NO: 38), or a nucleic acid encoding a Nmb protein (such as a nucleic acid molecule having at least 50%, at least 60%, at least 70%, at least 80%, at least 90%, at least 95%, at least 96%, at least 97%, at least 98%, at least 99% or 100% sequence identity to SEQ ID NO: 2, or a nucleic acid molecule that encodes a protein having at least 50%, at least 60%, at least 70%, at least 80%, at least 90%, at least 95%, at least 96%, at least 97%, at least 98%, at least 99% or 100% sequence identity to SEQ ID NO: 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37) or a Nme coding sequence (such as a nucleic acid molecule that encodes a protein having at least 50%, at least 60%, at least 70%, at least 80%, at least 90%, at least 95%, at least 96%, at least 97%, at least 98%, at least 99% or 100% sequence identity to SEQ ID NO: 38) can be dissolved in a carrier, such as saline, and atomized using the devices above. The associated aerosols can be collected using a NEXT GENERATION IMPACTOR® (NGI) (MSP Corp., Shoreview, MN), which uses a series of aerodynamic stages to separate and collect the aerosol into separate fractions based on droplet size, such as those that will deposit in the small airways and alveoli.

Aerosol particle size is often expressed in terms of mass median aerodynamic diameter (MMAD), a parameter that is based on particle size, shape, and density. For a spherical particle, MMAD is equal to MMD (p^{1/2}), in which MMD is mass median diameter and r is the bulk density. For a non-spherical particle, MMAD is equal to MMD (p/x)^{1/2}, in which X is the shape factor. Thus, particles with larger than unit density will have actual diameters smaller than their MMAD.

The site of particle deposition within the respiratory tract is demarcated based on particle size. In one example, particles of about 1 to about 500 microns are utilized, such as particles of about 25 to about 250 microns, or about 10 to about 25 microns are utilized. In other embodiments, particles of about 1 to 50 microns are utilized. For use in a metered dose inhaler, for administration to lungs particles of less than about 10 microns, such as particles of about 2 to about 8 microns, such as about 1 to about 5 microns, such as particles of 2 to 3 microns, can be utilized.

The dosage form of the pharmaceutical composition can be determined by the mode of administration chosen. For instance, in addition to injectable fluids, topical, inhalation, oral and suppository formulations can be employed. Topical preparations can include eye drops, ointments, sprays, patches and the like. Inhalation preparations can be liquid (e.g., solutions or suspensions) and include mists, sprays and the like. Oral formulations can be liquid (e.g., syrups, solutions or suspensions), or solid (e.g., powders, pills, tablets, or capsules). Suppository preparations can also be solid, gel, or in a suspension form. For solid compositions, conventional non-toxic solid carriers can include pharmaceutical grades of mannitol, lactose, cellulose, starch, or magnesium stearate.

In some examples, the therapeutically effective amount of Nmb protein (such as a protein having at least 50%, at least 60%, at least 70%, at least 80%, at least 90%, at least 95%, at least 96%, at least 97%, at least 98%, at least 99% or 100% sequence identity to SEQ ID NO: 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37) or Nme protein (such as a protein having at least 50%, at least 60%, at least 70%, at least 80%, at least 90%, at least 95%, at least 96%, at least 97%, at least 98%, at least 99% or 100% sequence identity to SEQ ID NO: 38) or a Nmb coding sequence (such as a nucleic acid molecule having at least 50%, at least 60%, at least 70%, at least 80%, at least 90%, at least 95%, at least 96%, at least 97%, at least 98%, at least 99% or 100% sequence identity to SEQ ID NO: 2) or Nme coding sequence (such as a nucleic acid molecule that encodes a protein having at least 50%, at least 60%, at least 70%, at least 80%, at least 90%, at least 95%, at least 96%, at least 97%, at least 98%, at least 99% or 100% sequence identity to SEQ ID NO: 38) is the amount of the Nmb or Nme protein or nucleic acid encoding Nmb or Nme that is necessary to (1) decrease inflammation, for example in the lung or at the site of an allergic reaction, such as decrease of at least 5%, at least 10%, at least 15%, at least 20%, at least 30%, at least 40%, at least 50%, at least 60%, at least 75%, or at least 90% for example relative to no administration of the Nmb or Nme protein or nucleic acid molecule, (2) decrease IL-5, for example in ILC2s and/or T cells, such as decrease of at least 5%, at least 10%, at least 15%, at least 20%, at least 30%, at least 40%, at least 50%, at least 60%, at least 75%, or at least 90% for example relative to no administration of the Nmb or Nme protein or nucleic acid molecule, (3) decrease IL-13, for example in ILC2s and/or T cells, such as decrease of at least 5%, at least 10%, at least 15%, at least 20%, at least 30%, at least 40%, at least 50%, at least 60%, at least 75%, or at least 90% for example relative to no administration of the Nmb or Nme protein or nucleic acid molecule, (4) decrease ILC2 and/or T responses, for example numbers of present, proliferating and/or activated ILC2s and/or T cells, such as decrease of at least 5%, at least 10%, at least 15%, at least 20%, at least 30%, at least 40%, at least 50%, at least 60%, at least 75%, or at least 90% for example relative to no administration of the Nmb or Nme protein or nucleic acid molecule, and/or (5) decrease eosinophilia, for example in the lung or in the peripheral blood, such as decrease of at least 5%, at least 10%, at least 15%, at least 20%, at least 30%, at least 40%, at least 50%, at least 60%, at least 75%, or at least 90% for example relative to no administration of the Nmb or Nme protein or nucleic acid molecule.

The pharmaceutical compositions that include a Nmb protein (such as a protein having at least 50%, at least 60%, at least 70%, at least 80%, at least 90%, at least 95%, at least 96%, at least 97%, at least 98%, at least 99% or 100% sequence identity to SEQ ID NO: 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37) or Nme protein (such as a protein having at least 50%, at least 60%, at least 70%, at least 80%, at least 90%, at least 95%, at least 96%, at least 97%, at least 98%, at least 99% or 100% sequence identity to SEQ ID NO: 38) can be formulated in unit dosage form, suitable for individual administration of precise dosages. In one non-limiting example, a unit dosage contains from about 1 μg to about 1 g of a Nmb protein (such as a protein having at least 50%, at least 60%, at least 70%, at least 80%, at least 90%, at least 95%, at least 96%, at least 97%, at least 98%, at least 99% or 100% sequence identity to SEQ ID NO: 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37) or Nme protein (such as a protein having at least 50%, at least 60%, at least 70%, at least 80%, at least 90%, at least 95%, at least 96%, at least 97%, at least 98%, at least 99% or 100% sequence identity to SEQ ID NO: 38), such as about 1 mg to 100 mg, 10 mg to about 100 mg, about 50 mg to about 500 mg, about 50 mg to about 100 mg, about 100 mg to about 900 mg, about 250 mg to about 750 mg, or about 400 mg to about 600 mg. In other examples, a therapeutically effective amount of a Nmb protein (such as a protein having at least 50%, at least 60%, at least 70%, at least 80%, at least 90%, at least 95%, at least 96%, at least 97%, at least 98%, at least 99% or 100% sequence identity to SEQ ID NO: 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37) or Nme protein (such as a protein having at least 50%, at least 60%, at least 70%, at least 80%, at least 90%, at least 95%, at least 96%, at least 97%, at least 98%, at least 99% or 100% sequence identity to SEQ ID NO: 38) is about 0.01 mg/kg to about 50 mg/kg, for example, about 0.1 mg/kg to about 10 mg/kg, about 0.1 mg/kg to about 1 mg/kg, or about 1 mg/kg to about 10 mg/kg. In other examples, a therapeutically effective amount of a Nmb protein (such as a protein having at least 50%, at least 60%, at least 70%, at least 80%, at least 90%, at least 95%, at least 96%, at least 97%, at least 98%, at least 99% or 100% sequence identity to SEQ ID NO: 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37) or Nme protein (such as a protein having at least 50%, at least 60%, at least 70%, at least 80%, at least 90%, at least 95%, at least 96%, at least 97%, at least 98%, at least 99% or 100% sequence identity to SEQ ID NO: 38) is about 0.1 ug/kg to about 10 μg/kg, about 0.1 ug/kg to about 1 μg/kg, for example about 0.8 ug/kg. In a particular example, a therapeutically effective amount of a Nmb protein (such as a protein having at least 50%, at least 60%, at least 70%, at least 80%, at least 90%, at least 95%, at least 96%, at least 97%, at least 98%, at least 99% or 100% sequence identity to SEQ ID NO: 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37) or Nme protein (such as a protein having at least 50%, at least 60%, at least 70%, at least 80%, at least 90%, at least 95%, at least 96%, at least 97%, at least 98%, at least 99% or 100% sequence identity to SEQ ID NO: 38) is about 1 mg/kg to about 10 mg/kg, such as about 2 mg/kg.

Other suitable ranges include doses of Nmb or Nme protein from about 100 g/kg to 10 mg/kg body weight or more (such as about 0.1-10 mg/kg, about 1-20 mg/kg, about 5-50 mg/kg, or about 10-100 mg/kg). In certain embodiments, the effective dosage will be selected within narrower ranges of, for example, 5-40 mg/kg, 10-35 mg/kg or 20-25 mg/kg. In other examples, the dosage is about 1-100 mg, such as about 1-10 mg, about 5-25 mg, about 10-50 mg, about 25-60 mg, or about 50-100 mg (for example, about 1 mg, 5, mg, 10 mg, 15 mg, 20 mg, 25 mg, 30 mg, 35 mg, 40 mg, 45 mg, 50 mg, 55 mg, 60 mg, 70 mg, 80 mg, 90 mg, or 100 mg). In particular examples, the dose is about 20-60 mg, and in one non-limiting example, about 25 mg.

Pharmaceutical compositions that include a Nmb coding sequence (such as one encoding a protein having at least 50%, at least 60%, at least 70%, at least 80%, at least 90%, at least 95%, at least 96%, at least 97%, at least 98%, at least 99% or 100% sequence identity to SEQ ID NO: 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37, such as a nucleic acid molecule having at least 50%, at least 60%, at least 70%, at least 80%, at least 90%, at least 95%, at least 96%, at least 97%, at least 98%, at least 99% or 100% sequence identity to SEQ ID NO: 2) or a Nme coding sequence (such as a nucleic acid molecule that encodes a protein having at least 50%, at least 60%, at least 70%, at least 80%, at least 90%, at least 95%, at least 96%, at least 97%, at least 98%, at least 99% or 100% sequence identity to SEQ ID NO: 38) can be formulated in unit dosage form, suitable for individual administration of precise dosages. Generally, the quantity of recombinant viral vector, carrying the nucleic acid coding sequence of Nmb protein to be administered, is based on the titer of virus particles. In one non-limiting example, for example when a viral vector is utilized for administration of a nucleic acid encoding an Nmb protein (such as a protein having at least 50%, at least 60%, at least 70%, at least 80%, at least 90%, at least 95%, at least 96%, at least 97%, at least 98%, at least 99% or 100% sequence identity to SEQ ID NO: 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37) or a Nme coding sequence (such as a nucleic acid molecule that encodes a protein having at least 50%, at least 60%, at least 70%, at least 80%, at least 90%, at least 95%, at least 96%, at least 97%, at least 98%, at least 99% or 100% sequence identity to SEQ ID NO: 38), such as a vector containing a nucleic acid molecule having at least 50%, at least 60%, at least 70%, at least 80%, at least 90%, at least 95%, at least 96%, at least 97%, at least 98%, at least 99% or 100% sequence identity to SEQ ID NO: 2), a unit dosage (e.g., 0.5-1.5 μl) contains about 10⁵ to about 10¹⁰ plaque forming units (pfu)/ml per mammal. Thus, in some examples, the recipient subject is administered a dose of about 105 to about 10¹⁰ pfu/ml per mammal of recombinant virus in the composition. In some examples, the recipient subject is administered a dose of at least 10⁵ pfu/ml per mammal, at least 10⁶ pfu/ml per mammal at least 10⁷ pfu/ml per mammal at least 10⁸ pfu/ml per mammal, at least 10⁹ pfu/ml per mammal, or at least 10¹⁰ pfu/ml per mammal. Examples of methods for administering the composition into mammals include, but are not limited to, injection of the composition into the affected tissue (such as into the skin or lung) or intravenous, subcutaneous, intradermal or intramuscular administration.

The compositions of this disclosure that include a Nmb protein (such as a protein having at least 50%, at least 60%, at least 70%, at least 80%, at least 90%, at least 95%, at least 96%, at least 97%, at least 98%, at least 99% or 100% sequence identity to SEQ ID NO: 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37) or Nme protein (such as a protein having at least 50%, at least 60%, at least 70%, at least 80%, at least 90%, at least 95%, at least 96%, at least 97%, at least 98%, at least 99% or 100% sequence identity to SEQ ID NO: 38) or a Nmb coding sequence (such as a nucleic acid molecule having at least 50%, at least 60%, at least 70%, at least 80%, at least 90%, at least 95%, at least 96%, at least 97%, at least 98%, at least 99% or 100% sequence identity to SEQ ID NO: 2) or a Nine coding sequence (such as a nucleic acid molecule that encodes a protein having at least 50%, at least 60%, at least 70%, at least 80%, at least 90%, at least 95%, at least 96%, at least 97%, at least 98%, at least 99% or 100% sequence identity to SEQ ID NO: 38) can be administered to humans or other animals by any means, including orally, intravenously, intramuscularly, intraperitoneally, intranasally, intradermally, intraparenchymally, intracerebroventricularly, intrathecally (e.g., cisternal and lumbar), subcutaneously, via inhalation or via suppository. In one non-limiting example, the composition is administered via injection. In some examples, site-specific administration of the composition can be used, for example by administering the Nmb or Nme protein or coding sequence to skin tissue or into the lung (e.g., via inhalation).

Treatment can involve a single administration, or multiple administrations (such as at least two separate administrations), such as doses over a period of a few days to months, or even years. For example, a therapeutically effective amount of Nmb protein (such as a protein having at least 50%, at least 60%, at least 70%, at least 80%, at least 90%, at least 95%, at least 96%, at least 97%, at least 98%, at least 99% or 100% sequence identity to SEQ ID NO: 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37) or Nme protein (such as a protein having at least 50%, at least 60%, at least 70%, at least 80%, at least 90%, at least 95%, at least 96%, at least 97%, at least 98%, at least 99% or 100% sequence identity to SEQ ID NO: 38), or a Nmb coding sequence (such as one having at least 50%, at least 60%, at least 70%, at least 80%, at least 90%, at least 95%, at least 96%, at least 97%, at least 98%, at least 99% or 100% sequence identity to SEQ ID NO: 2) or a Nme coding sequence (such as a nucleic acid molecule that encodes a protein having at least 50%, at least 60%, at least 70%, at least 80%, at least 90%, at least 95%, at least 96%, at least 97%, at least 98%, at least 99% or 100% sequence identity to SEQ ID NO: 38) can be administered in a single dose, or in several doses, for example daily, weekly, monthly, or yearly, during a course of treatment. In a particular non-limiting example, treatment involves administration once monthly, once yearly, or every-other-month. In some examples, where multiple doses are administered, the at least two separate administrations can be separated by at least 1 week, at least 2 weeks, at least 3 weeks, at least 4 weeks, at least 1 month, at least 2 months, at least 3 months, at least 6 months, at least 9 months, or at least one year.

In some examples, the first dose (and in some examples the only dose) administrated occurs within 1 minute, within 10 minutes, within 15 minutes, within 30 minutes, within 1 hour, within 2 hours, within 3 hours, within 4 hours, within 5 hours, within 6 hours, within 12 hours, within 24 hours, within 48 hours, within 72 hours, within 96 hours, within 1 week, within 2 weeks, within 3 weeks, within 4 weeks, within 1 month, within 2 months, or within 3 months of the onset of the disorder (such as type 2 cytokine inflammation), such as within 1 to 24 hours, 2 to 24 hours, 4 to 24 hours, or 1 to 96 hours of the onset of the disorder.

In some embodiments, the Nmb protein (such as a protein having at least 50%, at least 60%, at least 70%, at least 80%, at least 90%, at least 95%, at least 96%, at least 97%, at least 98%, at least 99% or 100% sequence identity to SEQ ID NO: 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37) or Nme protein (such as a protein having at least 50%, at least 60%, at least 70%, at least 80%, at least 90%, at least 95%, at least 96%, at least 97%, at least 98%, at least 99% or 100% sequence identity to SEQ ID NO: 38) or a Nmb coding sequence (such as a nucleic acid molecule having at least 50%, at least 60%, at least 70%, at least 80%, at least 90%, at least 95%, at least 96%, at least 97%, at least 98%, at least 99% or 100% sequence identity to SEQ ID NO: 2) or a Nme coding sequence (such as a nucleic acid molecule that encodes a protein having at least 50%, at least 60%, at least 70%, at least 80%, at least 90%, at least 95%, at least 96%, at least 97%, at least 98%, at least 99% or 100% sequence identity to SEQ ID NO: 38) is administered prophylactically prior to exposure to an allergic trigger. For example, patients with asthma can be treated with an Nmb or Nme protein or nucleic acid molecule prior to exercise or prior to exposure to an environmental trigger, such as pollution or an allergen. Prophylactic treatment can also be useful for treating subjects at risk for chemical exposure, such as first responders to a chemical accident. The Nmb or Nme protein or nucleic acid molecule can also be administered prophylactically to asymptomatic individuals who are repeatedly exposed to agents known to trigger as asthmatic episode in the subject. In one example, an effective amount of the Nmb or Nme protein or nucleic acid molecule can be administered to a healthy individual who is repeatedly exposed to an allergen known to induce asthmatic episode. In other embodiments, the Nmb or Nme protein or nucleic acid molecule is administered to a subject that requires corticosteroid (CS) therapy, or who has had previous CS therapy. The Nmb or Nme protein or nucleic acid molecule can be administered to a patient suffering from asthma prior to partaking in activities which trigger asthma attacks to lessen the severity of, or avoid altogether, an asthmatic episode. Thus, in some embodiments, administration of the Nmb or Nme protein or nucleic acid molecule prevents an asthmatic episode. In some embodiments, the Nmb or Nme protein or nucleic acid molecule is administered to improve symptoms chronically in those who suffer daily symptoms.

In some examples, effectiveness is measured by detecting or measuring (1) decreased inflammation, for example in the lung or at the site of an allergic reaction, such as decrease of at least 5%, at least 10%, at least 15%, at least 20%, at least 30%, at least 40%, at least 50%, at least 60%, at least 75%, or at least 90% for example relative to no administration of the Nmb or Nme protein or nucleic acid molecule, (2) decreased IL-5, for example in ILC2s and/or T cells, such as decrease of at least 5%, at least 10%, at least 15%, at least 20%, at least 30%, at least 40%, at least 50%, at least 60%, at least 75%, or at least 90% for example relative to no administration of the Nmb or Nme protein or nucleic acid molecule, (3) decreased IL-13, for example in ILC2s and/or T cells, such as decrease of at least 5%, at least 10%, at least 15%, at least 20%, at least 30%, at least 40%, at least 50%, at least 60%, at least 75%, or at least 90% for example relative to no administration of the Nmb or Nme protein or nucleic acid molecule, (4) decrease ILC2 and/or T responses, for example numbers of present, proliferating and/or activated ILC2s and/or T cells, such as decrease of at least 5%, at least 10%, at least 15%, at least 20%, at least 30%, at least 40%, at least 50%, at least 60%, at least 75%, or at least 90% for example relative to no administration of the Nmb or Nme protein or nucleic acid molecule, and/or (5) decreased eosinophilia, for example in the lung or in the peripheral blood, such as decrease of at least 5%, at least 10%, at least 15%, at least 20%, at least 30%, at least 40%, at least 50%, at least 60%, at least 75%, or at least 90% for example relative to no administration of the Nmb or Nme protein or nucleic acid molecule.

In some examples, the effectiveness of treatment is measured by monitoring pulmonary function. For example, various measurable parameters of lung function can be studied before, during, or after treatment. Pulmonary function can be monitored by testing any of several physically measurable operations of a lung including, but not limited to, inspiratory flow rate, expiratory flow rate, and lung volume. A statistically significant increase in one or more of these parameters indicates efficacy of the treatment. A decrease in symptoms, asthma exacerbations, use of rescue inhalers or measures of inflammation are also evidence of efficacy.

The methods of measuring pulmonary function most commonly employed in clinical practice involve timed measurement of inspiratory and expiratory maneuvers to measure specific parameters. For example, FVC measures the total volume in liters exhaled by a patient forcefully from a deep initial inspiration. This parameter, when evaluated in conjunction with the FEV1, allows bronchoconstriction to be quantitatively evaluated. A statistically significant increase, as determined by mathematical formulas in FVC or FEV1 reflects a decrease in bronchoconstriction, and indicates that therapy is effective.

In addition to measuring volumes of exhaled air as indices of pulmonary function, the flow in liters per minute measured over differing portions of the expiratory cycle can be useful in determining the status of a patient's pulmonary function. In particular, the peak expiratory flow, taken as the highest airflow rate in liters per minute during a forced maximal exhalation, is well correlated with overall pulmonary function in a patient with asthma and other respiratory diseases. Thus, a statistically significant increase in the peak expiratory flow following administration of a TPO inhibitor, indicates that the therapy is effective.

### F. Administration of Additional Therapy

In some examples, the Nmb protein (such as a protein having at least 50%, at least 60%, at least 70%, at least 80%, at least 90%, at least 95%, at least 96%, at least 97%, at least 98%, at least 99% or 100% sequence identity to SEQ ID NO: 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37) or Nme protein (such as a protein having at least 50%, at least 60%, at least 70%, at least 80%, at least 90%, at least 95%, at least 96%, at least 97%, at least 98%, at least 99% or 100% sequence identity to SEQ ID NO: 38) or a Nmb coding sequence (such as one having at least 50%, at least 60%, at least 70%, at least 80%, at least 90%, at least 95%, at least 96%, at least 97%, at least 98%, at least 99% or 100% sequence identity to SEQ ID NO: 2) or Nme coding sequence (such as a nucleic acid molecule that encodes a protein having at least 50%, at least 60%, at least 70%, at least 80%, at least 90%, at least 95%, at least 96%, at least 97%, at least 98%, at least 99% or 100% sequence identity to SEQ ID NO: 38), is administered in combination (such as sequentially, simultaneously, or contemporaneously) with one or more other agents, such as those useful in the treatment of an inflammatory disorder or other disorder listed in Table 1. The term “administration in combination” or “co-administration” refers to both concurrent and sequential administration of the active agents.

In some examples, a Nmb or Nme protein or Nmb or Nme coding sequence is administered to a subject in combination with an effective dose of a corticosteroid (such as methylprednisolone or prednisone), antihistamine, or both. In some examples, a Nmb protein or Nmb coding sequence is administered to a subject in combination with an effective dose of an IL-4 inhibitor (e.g., dupilumab), an IL-5 inhibitor (e.g., mepolizumab, benralizumab, and reslizumab), an IL-13 inhibitor (e.g., tralokinumab and lebrikizumab), or combinations thereof. In another example, a Nmb or Nme protein or Nmb or Nme coding sequence is administered to a subject in combination with an effective dose of PGE2.

In some examples, a Nmb or Nme protein or Nmb or Nme coding sequence is administered to a subject in combination with an effective dose of an agent for preventing or treating one or more signs or symptoms associated with an airway disorder (such as asthma or COPD). For example, one or more (3-agonists, including a beta-2 agonist (such as salbutamol), one or more leukotriene antagonists/formation inhibitors (for example, zileuton, ZYFLO®, Abbott Laboratories, monteleukast, SINGULAIR®, Merck and Company, and others), an antibody which blocks IgE, an expectorant, or combinations thereof, can also be administered.

In some examples, a Nmb or Nme protein or Nmb or Nme coding sequence is administered to a subject with an inflammatory disorder, such as an allergy or allergic reaction, sinusitis, asthma (mild, moderate, or severe, including eosinophilic asthma), COPD, idiopathic pulmonary fibrosis (IPF), rhinitis, EGPA, eosinophilic esophagus, eczema, urticarial, chronic itch, angioedema, conjunctivitis or atopic dermatitis, in combination with effective doses of one or more other therapeutic agents. For example, if the subject has had an allergic reaction (for example a seasonal allergy, or allergic reaction to dust/mold, food (e.g., shellfish, eggs, milk, nuts, wheat), animal (e.g., dander), plans, drug (e.g., antibiotics such as sulfa or penicillin, aspirin, NSAIDs, anticonvulsants, chemotherapy drugs), insect (e.g., cockroaches, as well as venom from honeybees, hornets, wasps, yellow jackets, fire ants), pathogen (e.g., virus, bacterium, fungus, parasite), or chemical (e.g., latex), the method can further include administering a therapeutically effective amount of one or more of a decongestant, antihistamine, corticosteroid, immunotherapy and epinephrine. For example, if the subject has sinusitis, the method can further include administering a therapeutically effective amount of an antibiotic (e.g., amoxicillin) and/or a corticosteroid. For example, if the subject has asthma (mild, moderate, or severe, including eosinophilic asthma), the method can further include administering a therapeutically effective amount of one or more of a corticosteroid, glucocorticoid, bronchodilator (such as short (e.g., salbutamol) or long acting (e.g., formoterol) beta-2 adrenergic agonist, anticholinergic (e.g., tiotropium and ipratropium bromide), long acting beta agonist (LABA) leukotriene modifier, an IL-4 inhibitor (e.g., dupilumab), mepolizumab, benralizumab, reslizumab, tralokinumab, and lebrikizumab. For example, if the subject has COPD, the method can further include administering a therapeutically effective amount of one or more of a corticosteroid, bronchodilator (such as short (e.g., salbutamol) or long acting (e.g., formoterol) beta-2 adrenergic agonist, anticholinergic (e.g., tiotropium and ipratropium bromide), antibiotic (e.g., erthyromycin) and supplemental oxygen. For example, if the subject has idiopathic pulmonary fibrosis (IPF), the method can further include administering a therapeutically effective amount of Pirfenidone and/or a angiokinase inhibitor (e.g., Nintedanib). For example, if the subject has rhinitis, the method can further include administering a therapeutically effective amount of one or more of an antihistamine nasal spray, a corticosteroid nasal spray, anticholingeric nasal spray (e.g., ipratropium), and a decongestant (e.g., pseudoephedrine or phenylephrine). For example, if the subject has EGPA, the method can further include administering a therapeutically effective amount of one or more of mepolizumab, a glucocorticoid (e.g., prednisolone), an immunosuppressant (e.g., azathioprine and cyclophosphamide) and methotrexate. For example, if the subject has eosinophilic esophagitis, the method can further include administering a therapeutically effective amount of a topical corticosteroid (e.g., budesonide, fluticasone) and/or a proton pump inhibitor (e.g., omeprazole, lansoprazole, dexlansoprazole, esomeprazole, pantoprazole, rabeprazole, ilaprazole). For example, if the subject has eczema, the method can further include administering a therapeutically effective amount of a corticosteroid (e.g., hydrocortisone, clobetasol propionate) and/or an immunosuppressant (e.g., pimecrolimus, tacrolimus). For example, if the subject has urticaria, the method can further include administering a therapeutically effective amount of one or more of an antihistamine (e.g., diphenhydramine, hydroxyzine, loratadine, cetirizine, desloratadine), leukotriene antagonist (e.g., montelukast and zafirlukast), oral glucocorticoid, anti-inflammatory, omalizumab, and an immunosuppressant. For example, if the subject has chronic itch, the method can further include administering a therapeutically effective amount of one or more of corticosteroids (e.g., cortisone and prednisone), calcineurin inhibitors (e.g., pimecrolimus and tucrolimus) and antidepressants (e.g., Prozac and Zoloft). For example, if the subject has angioedema, the method can further include administering a therapeutically effective amount of an antihistamine (e.g., cetirizine) and/or an androgen. For example, if the subject has conjunctivitis, the method can further include administering a therapeutically effective amount of one or more of an antihistamine (e.g., diphenhydramine), mast cell stabilizer (e.g., cromolyn), or antibiotic. For example, if the subject has atopic dermatitis, the method can further include administering a therapeutically effective amount of one or more of tralokinumab, topical corticosteroid (e.g., hydrocortisone), topical calcineurin inhibitor (e.g., tacrolimis or pimecrolimus), and systemic immunosuppressant (e.g., ciclospoin, methotrexate, interferon gamma-1b). For example, if the subject has an eosinophilic disorder (e.g., eosinophilic esophagitis, eosinophilic gastritis, eosinophilic gastroenteritis, eosinophilic enteritis, eosinophilic colitis, eosinophilic asthma, eosinophilic gastrointestinal disorder (EGID), eosinophilic fasciitis, EGPA, eosinophilic lung disorder, hypereosinophilic syndrome (HES)), the method can further include administering a therapeutically effective amount of one or more of topical or systemic steroids (e.g., corticosteroid, glucocorticoid, such as prednisone), amino acid based diet, and an immunosuppressant (e.g., azathioprine and cyclophosphamide). In some examples, a Nmb or Nme protein or Nmb or Nme coding sequence is administered to a subject with a parasitic or fungal infection, and the method can further include administering a therapeutically effective amount of an antifungal agent (e.g., polyene (e.g. amphotericin B, nystatin, natamycin), azole (e.g., fluconazole, itraconazole, voriconazole), allylamine (e.g., terbinafine), and echinocandin (e.g. caspofungin) and/or an anti-parasitic agent (e.g., anthelmintic, antiprotozoal, antiamebic).

In some examples, a Nmb or Nme protein or Nmb or Nme coding sequence is administered to a subject with eosinophilic leukemia (chronic, acute, or clonal), and the method can further include administering a therapeutically effective amount of one or more of chemotherapy (e.g., cytarabine, anthracycline, histamine dihydrochloride, interleukin 2), Gleevec, tyrosine kinase inhibitor (e.g., sorafenib, midostaruin, ponatinib), and hematopoietic stem cell transplant.

In some examples, a Nmb or Nme protein or Nmb or Nme coding sequence is administered to a subject with Hodgkin's lymphoma, and the method can further include administering a therapeutically effective amount of one or more of lebrikizumab, MOPP, radiation therapy, ABVD (adriamycin, bleomycin, vinblastine, dacarbazine), Stanford V (adriamycin, bleomycin, vinblastine, vincristine, chlormethine, etoposide, prednisone), and BEACOPP (doxorubicin, bleomycin, vincristine, cyclophosphamide, procarbazine, etoposide, prednisone).

### IV. Compositions

Also provided are compositions, which can be used with the disclosed methods. In one example, the composition includes an isolated Nmb protein having at least 50%, at least 60%, at least 70%, at least 80%, at least 85%, at least 90%, at least 95%, at least 98%, at least 99%, or 100% sequence identity to SEQ ID NO: 1 or an isolated Nme protein having at least 50%, at least 60%, at least 70%, at least 80%, at least 85%, at least 90%, at least 95%, at least 98%, at least 99%, or 100% sequence identity to SEQ ID NO: 38 and a liposome, wherein the Nmb or Nme protein is encapsulated in the liposome. In one example, the composition includes a Nmb fusion protein composed of (1) a Nmb protein having at least 50%, at least 60%, at least 70%, at least 80%, at least 85%, at least 90%, at least 95%, at least 98%, at least 99%, or 100% sequence identity to SEQ ID NO: 1 or a Nme protein having at least 50%, at least 60%, at least 70%, at least 80%, at least 85%, at least 90%, at least 95%, at least 98%, at least 99%, or 100% sequence identity to SEQ ID NO: 38 and (2) a cell penetrating peptide (or a nucleic acid molecule encoding such a fusion protein). In one example, the composition includes an isolated non-native Nmb protein comprising at least 50%, at least 60%, at least 70%, at least 80%, at least 85%, at least 90%, at least 95%, at least 98%, at least 99%, or 100% sequence identity to SEQ ID NO: 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37 (that is not SEQ ID NO: 1), a pharmaceutically acceptable carrier, and optionally a liposome. In one example, the composition includes at least one non-native Nmb protein, such as one having at least 50%, at least 60%, at least 70%, at least 80%, at least 90%, or 100% sequence identity to SEQ ID NO: 3, 4, 5, 6, 11, 12, 13, 14, 20, 22, 26, 27, 28, 29, 30, 31, 32, 35, 36 or 37, that is not SEQ ID NO: 1; and/or one having at least 50%, at least 60%, at least 70%, at least 80%, at least 90%, or 100% sequence identity to SEQ ID NO: 26, 27, 29, 35 or 36, that is not SEQ ID NO: 1. In one example, the composition includes (1) a native Nmb protein (e.g., SEQ ID NO: 1) and (2) a non-native Nmb protein, such as one comprising at least 50%, at least 60%, at least 70%, at least 80%, at least 85%, at least 90%, at least 95%, at least 98%, at least 99%, or 100% sequence identity to SEQ ID NO: 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37 (that is not SEQ ID NO: 1). In one example, the composition includes (1) one or more of SEQ ID NOS: 3, 4, 6, 7, 9, 10, 11, 12, 14, 15, and 19 and (2) one or more of SEQ ID NOS: 3, 4, 5, 6, 9, 11, 12, 13, 14, 18, 19, 20, 21, 22, and 23 (wherein the composition can further include a native Nmb protein, such as SEQ ID NO: 1).

In one example, the composition includes an isolated non-native Nmb protein having at least 50%, at least 60%, at least 70%, at least 80%, at least 85%, at least 90%, at least 95%, at least 98%, at least 99%, or 100% sequence identity to SEQ ID NO: 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37 (e.g., that is not SEQ ID NO: 1) and a pharmaceutically acceptable carrier (such as water or saline). In some example, the non-native Nmb protein is encapsulated in a liposome. In some example, the non-native Nmb protein is part of a fusion protein composed of (1) a non-native Nmb protein having at least 50%, at least 60%, at least 70%, at least 80%, at least 85%, at least 90%, at least 95%, at least 98%, at least 99%, or 100% sequence identity to SEQ ID NO: 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37 and (2) a cell penetrating peptide (or a nucleic acid molecule encoding such a fusion protein). In some examples, the composition includes a non-native Nmb coding sequence in place of the non-native Nmb protein. In other examples, an Nme protein is part of a fusion protein composed of (1) an Nme protein having at least 50%, at least 60%, at least 70%, at least 80%, at least 85%, at least 90%, at least 95%, at least 98%, at least 99%, or 100% sequence identity to SEQ ID NO: 38 and (2) a cell penetrating peptide (or a nucleic acid molecule encoding such a fusion protein). Such compositions can further include other materials, such as a pharmaceutically acceptable carrier, such as water or saline. In some examples, the composition includes an Nmb or Nme coding sequence in place of the Nmb or Nme protein.

Exemplary cell penetrating peptides that can be used include hydrophilic peptides (e.g., TAT [YGRKKRRQRRR; SEQ ID NO: 57], SynB1 [RGGRLSYSRRRFSTSTGR; SEQ ID NO: 39], SynB3 [RRLSYSRRRF; SEQ ID NO: 40], PTD-4 [PIRRRKKLRRLK; SEQ ID NO: 41], PTD-5 [RRQRRTSKLMKR; SEQ ID NO: 42], FHV Coat-(35-49) [RRRRNRTRRNRRRVR; SEQ ID NO: 43], BMV Gag-(7-25) [KMTRAQRRAAARRNRWTAR; SEQ ID NO: 44], HTLV-II Rex-(4-16) [TRRQRTRRARRNR; SEQ ID NO: 45], D-Tat [GRKKRRQRRRPPQ; SEQ ID NO: 46], R9-Tat GRRRRRRRRRPPQ[SEQ ID NO: 47] and penetratin [RQIKWFQNRRMKWKK; SEQ ID NO: 48]), amphiphilic peptides (e.g., MAP [KLALKLALKLALALKLA; SEQ ID NO: 49], SBP [MGLGLHLLVLAAALQGAWSQPKKKRKV; SEQ ID NO: 50], FBP [GALFLGWLGAAGSTMGAWSQPKKKRKV; SEQ ID NO: 51], MPG ac-GALFLGFLGAAGSTMGAWSQPKKKRKV-cya; SEQ ID NO: 52], MPG(ΔNLS) [ac-GALFLGFLGAAGSTMGAWSQPKSKRKV-cya; SEQ ID NI: 53], Pep-2 [ac-KETWFETWFTEWSQPKKKRKV-cya; SEQ ID NO: 54], and transportan [GWTLNSAGYLLGKINLKALAALAKKIL; SEQ ID NI: 55]), periodic sequences (e.g., pVec, polyarginines R×N (4<N<17) chimera, polylysines K×N (4<N<17) chimera, (RAca)6R, (RAbu)6R, (RG)6R, (RM)6R, (RT)6R, (RS)6R, R10, (RA)6R, R7, and pep-1 [ac-KETWWETWWTEWSQPKKKRKV-cya; SEQ ID NO: 56]), Cr10 (a cyclic pol-arginine CPP), TAT₄₈₋₅₇, TAT₄₇₋₅₇, or TAT₄₉₋₅₇; penetratin; Pep-1; substance P, SP; polyarginines, such as R5-R12; pVEC; transportan; MAP; diatos peptide vector 1047, DPV1047, VECTOCELL®; MPG; ADP ribosylation factor, ARF, such as ARF₁₋₂₂; BPrPr (such as BPrPr₁₋₂₈); p28; VT5; Bac 7, such as Bac₁₋₂₄; C105Y; PFVYLI (SEQ ID NO: 58); and Pep-7.

In some examples, the composition is a liquid. In some examples, the composition is freeze dried or lyophilized.

Such pharmaceutical compositions can further include one or more diluents, fillers, binders, and other excipients, depending on the administration mode and dosage form. Examples of therapeutically inert inorganic or organic carriers include, but are not limited to, lactose, corn starch or derivatives thereof, talc, vegetable oils, waxes, fats, polyols such as polyethylene glycol, water, saccharose, alcohols, glycerin and the like. Various preservatives, emulsifiers, dispersants, flavorants, wetting agents, antioxidants, sweeteners, colorants, stabilizers, salts, buffers and the like can also be added.

In some examples, the composition furthers include one or more carriers, auxiliary substances, or stabilizers, such as a buffer (such as phosphate, citrate, tris or sodium acetate and other organic acids); antioxidant such as ascorbic acid; low molecular weight polypeptide (less than approximately 10 residues), protein such as serum albumin, gelatin or immunoglobulins; hydrophilic polymers such as polyvinylpyrrolidone; amino acids such as glycine, glutamine, asparagine, arginine, leucine or lysine; monosaccharides, disaccharides and other carbohydrates, for example glucose, sucrose, mannose, lactose, citrate, trehalose, maltodextrin or dextrin; chelating agents such as EDTA; sugar alcohols such as mannitol or sorbitol; salt-forming counter-ions such as sodium, and/or non-ionic surface-active substances such as Tween, Pluronics or polyethylene glycol (PEG).

In some examples, the composition is included in a nebulizer, a metered dose inhaler (MDI) or a dry powder inhaler (DPI). Formulations for dispensing from a powder inhaler device may include a finely divided dry powder containing the Nmb peptide or nucleic acid molecule can also include a bulking agent, such as lactose, sorbitol, sucrose, or mannitol in amounts which facilitate dispersal of the powder from the device, for example, 50 to 90% by weight of the formulation. The compound can be prepared in particulate form with an average particle size of less than 10⁻M, such as 0.5 to 5⁻M, for delivery to the distal lung (U.S. Patent Application Publication No. 2007/0065367).

The disclosure is illustrated by the following non-limiting Examples.

### Example 1

### Materials and Methods

This example provides the materials and methods used in the Examples below.

### Mice

8-10 week old C57BL(6) wild-type (WT), Mcpt8^{tm1(cre)Lksy}, ROSA26iDTR, and Rag2KO mice were purchased from The Jackson Laboratory. Basophil-depleted mice were obtained by crossing Mcpt8^{tm1(cre)Lksy} mice with ROSA26iDTR mice as previously described (Sullivan et al., Nat Immunol. 2011 June; 12(6):527-35, 2011). All mice were maintained in specific pathogen-free facilities.

N. brasiliensis Infection and Substance Administration

Methods for maintenance, recovery, infection and isolation of N. brasiliensis larvae were performed as previously described (Camberis et al., 2003, Animal model of Nippostrongylus brasiliensis and Heligmosomoides polygyrus. Current protocols in immunology/edited by John E. Coligan . . . [et al.] Chapter 19:Unit 19.12.). Mice were infected with ˜500 N. brasiliensis larvae by subcutaneous injection. For basophil depletion, WT and basophil-depleted mice were treated with 0.375 μg of diphtheria toxin i.p. every other day; mice were sacrificed 3-7 days post-N. brasiliensis infection. For neuromedin B treatment, mice were anesthetized and treated with 10 μg of neuromedin B (MP Biomedicals) dissolved in 50 μL of PBS administered via intratracheal instillation. For antibody-mediated basophil depletion, Rag2-deficient mice were treated with 20 μg of anti-FceR1 alpha antibody (clone MAR-1, eBioscience) or anti-CD200R3 antibody (clone Ba103, hycult biotech) i.p. on days 1, 3 and 5 post-N. brasiliensis infection.

### Adoptive Transfers

WT mice were injected (i.p.) with a combination of recombinant IL-3 (1 μg) and α-IL-3 antibody (0.5 μg) (BioLegend: clone MP2-8F8) in 200 μL of PBS every 3 days for 8 days. At necropsy, single cell suspensions of spleens were prepared and basophil populations were sort-purified. 15,000 basophils were resuspended in 50 μL of PBS and transferred into each mouse via intratracheal instillation on days 3, 4, 5 and 6 post-infection.

### Intravascular In Vivo Staining

Intravascular in vivo staining protocols were performed as previously described (Laidlaw et al., Immunity 41:633-645, 2014). Briefly, mice were injected with 3 μg of fluorescent-labeled antibody targeting CD200R (clone OX110, eBioscience) diluted in 300 μL of PBS, 5 minutes prior to euthanasia.

### Preparation of Lung and Bronchioalveolar Lavage (BAL) Cell Suspensions

For BAL collection after necropsy, 5 mL of PBS were injected and aspirated from the trachea of each mouse, after collection the volume of BAL collected was recorded. Following BAL collection, lungs were collected at necropsy and single-cell suspensions for flow cytometric analysis were prepared as previously described (Jungblut et al., J Vis Exp 29:1266, 2009). Briefly, pulmonary tissue was minced and incubated in HBSS containing 2.5% of FBS, collagenase D (2 mg/mL, Roche) and DNAse I (80 U/mL, Roche) for 30 min at 37° C. Cell suspensions were filtered through a 100 μM filter and analyzed by flow cytometry. Additionally sections of pulmonary tissue were collected for real-time PCR and histological analysis.

### Flow Cytometry and Cell Sorting

Cells were stained with monoclonal anti-mouse fluorescently conjugated antibodies: B220 (RA3-6B2), c-Kit (ACK2), CD3 (145-2C11), CD4 (GK1.5), CD5 (53-73), CD19 (1D3), NK1.1 (PK136), CD11b (MI/70), CD11c (N418), IgE (23G3), FcRI (MAR-1), CD49b (DX5), CD45 (30-F11), CD90 (5E10), CD127 (A7R34), F4/80 (BM8), γδTCR (eBioGL3), Siglec-F (E50-2440), LγδG (1A8), LγδC (AL-21), IL-5 (TRFK5), IL-13 (eBiol3A), Ter-119 (TER-119) from eBioscience or BD Biosciences. For intracellular staining, cells were incubated for 5 hours at 37° C. with Leukocyte Activation Cocktail, with BD GolgiPlug™ (BD Biosciences) following manufacturer's instructions. Basophils were analyzed as CD45⁺CD3⁻CD19⁻FceR1⁺CD49b⁺. Eosinophils were analyzed as CD45⁺CD11b⁺Siglec-F⁺CD11c⁻. Neutrophils were analyzed as Live CD45⁺CD11b⁺LγδG⁺. ILC2s were analyzed as CD45⁺CD3⁻CD19⁻CD11b⁻CD11c⁻NK1.1⁻B220⁻CD5⁻Ter-119⁻ γδ TCR⁻CD90⁺CD127⁺IL-5⁺IL-13⁺. Samples were acquired on a BD Fortessa flow cytometer (BD Biosciences) and analyzed using Flowlo software (v10.0.5, Tree Star). Cell sorting was performed using a FACSAriaII (BD Bioscience).

### ILC2 In Vitro Cultures

Lung cells were isolated from the lungs of WT or basophil-depleted mice on day 7 post-N. brasiliensis infection. ILC populations (CD45⁺Lin⁻CD90⁺CD127⁺) were sort-purified and 10,000 cells were cultured in the presence of 100 ng/mL of IL-2, IL-7 and vehicle (PBS) or 10 μg/mL of Neuromedin B for 24 hours. IL-5 and IL-13 were quantified in cell-free supernatants by ELISA.

### RNA Isolation and Quantitative Real-Time PCR Analysis

RNA from sections of lung tissue was isolated by homogenization in TRIzol (Invitrogen) followed by phenol-chloroform extraction and isopropanol precipitation. cDNA was generated per standard protocol with Superscript reverse transcriptase (Invitrogen) and used as input for real-time PCR. Real-time data were analyzed using the ΔΔCT method using SYBR Green chemistry (Applied Biosystems) with β-actin serving as the endogenous housekeeping gene. All reactions were run on an ABI 7500 Fast Real-Time PCR System (Applied Biosystems). Samples were normalized to naïve controls. The following QuantiTech primer assays from Qiagen were used: Mcpt1 (QT00157864), IL-4 (QT00160678), IL-5 (QT00099715), IL-13 (QT00099554), Mcpt8 (QT00131565), Nmb (QT00105945), Nmbr1 (QT00312494), Muc5ac (QT01161104).

### Pulse Oximetry.

Oxygen saturation was evaluated with the MouseOx Plus® (Starr Lifesciences Corp) following manufacturer's instructions. Briefly, the hair around the thigh was removed 1 day before N. brasiliensis infection, mice were anesthetized with 5% isoflurane and oxygen saturation was monitored using the thigh sensor for an interval of approximately 5 minutes.

### Statistics

Results are shown as mean±standard error of the mean. Statistical analysis was performed using Student's t-tests in GraphPad Prism version 6.

### Example 2

### Basophils Regulate Helminth-Induced Inflammation

The parameters of type 2 cytokine-mediated inflammation induced by Nb in the presence and absence of basophils was examined.

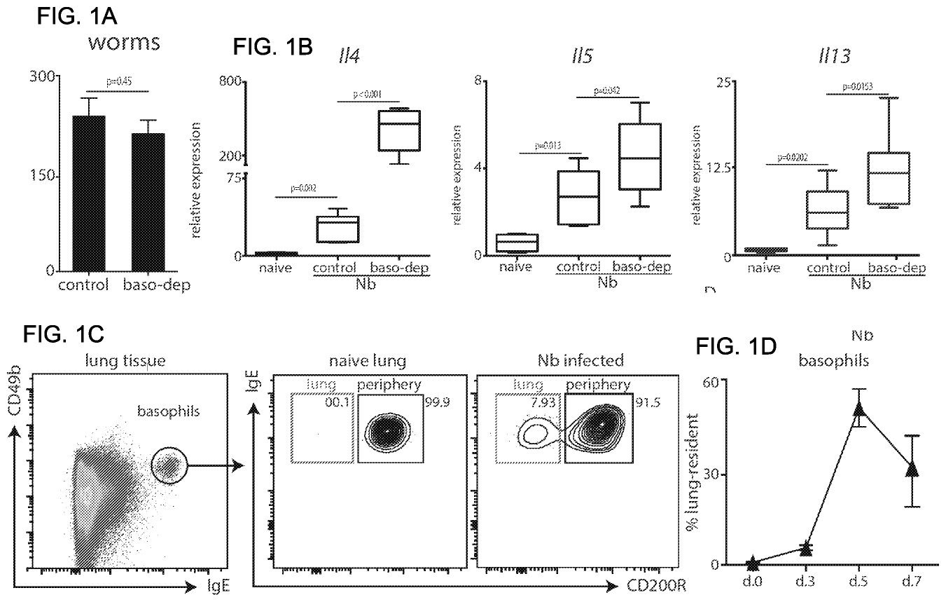

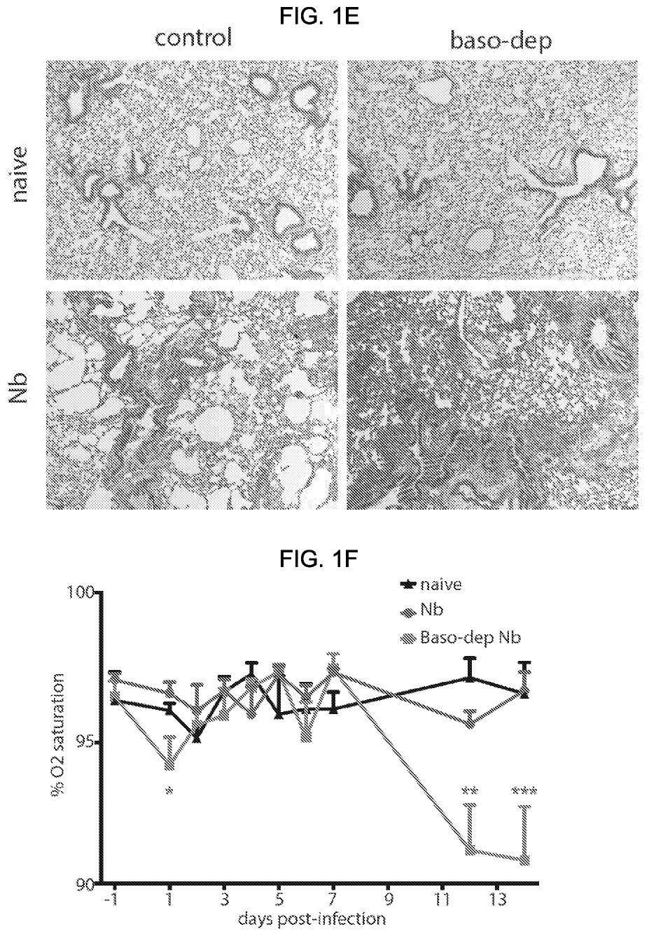

While lineage-specific depletion of basophils (11) did not alter inflammation in the gastrointestinal tract or worm expulsion (FIG. 1A), basophil depletion resulted in significantly increased type 2 cytokine responses in the lung (FIG. 1B). In vivo staining protocols used to distinguish blood-versus tissue-resident cells (12) revealed that tissue-resident basophil populations could be found in the lungs starting on day 3 and peaking on day 5 post-infection (FIGS. 1C,1D).

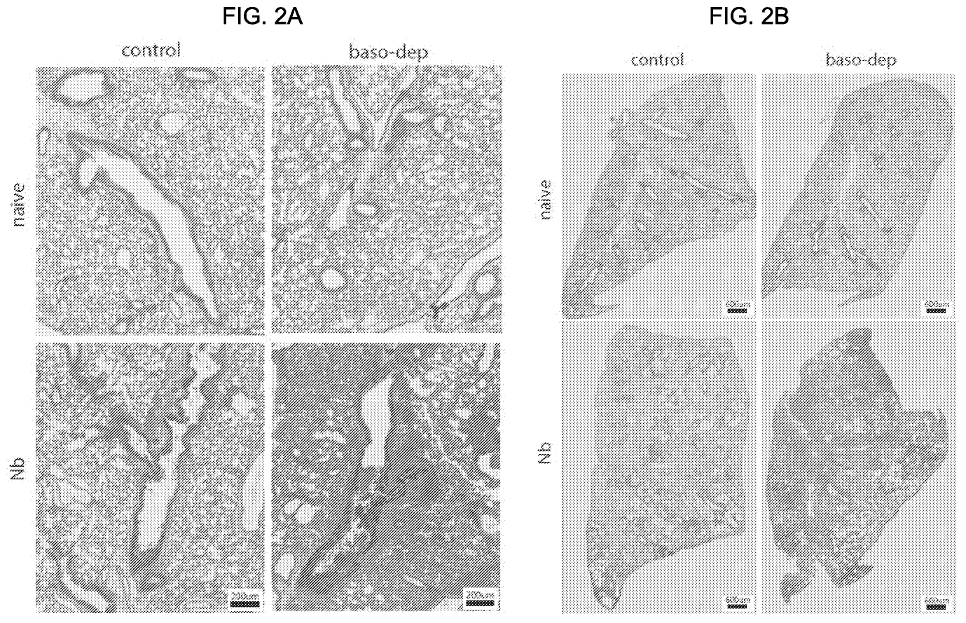

While effector cells activated in the context of type 2 cytokine responses are reported to promote parasite clearance, they also promote the integrity of parasite-affected tissues (2). Since Nb larvae exit the lung tissue on day 3 post-infection before the majority of basophils arrive, basophils may regulate inflammation in an attempt to restore lung function rather than serve to limit parasitic burdens. Supporting this hypothesis, Nb-infected mice depleted of basophils exhibited altered lung pathology marked by increased mucus production (FIG. 2A) and inflammatory cell infiltrates (FIGS. 1E, 2B) and had significantly reduced oxygen levels compared to control mice (FIG. 1F). Collectively, these data indicate that basophil populations negatively regulate infection-induced type 2 cytokine responses and assist in maintaining lung function post-infection.

### Example 3

### Basophils Negatively Regulate ILC2 Responses

The microscopic analysis of lung pathology observed in Example 2 indicated that basophil-depleted mice had elevated infection-induced eosinophil responses.

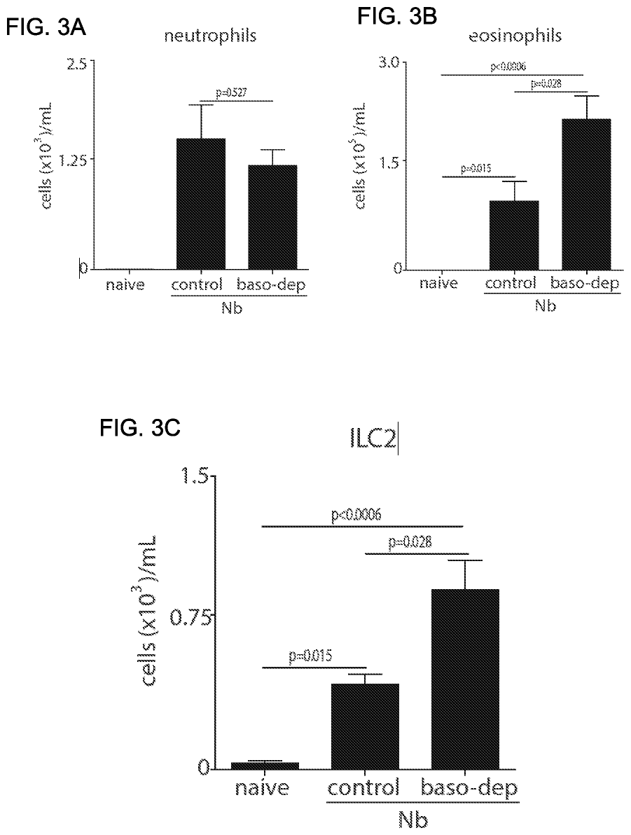

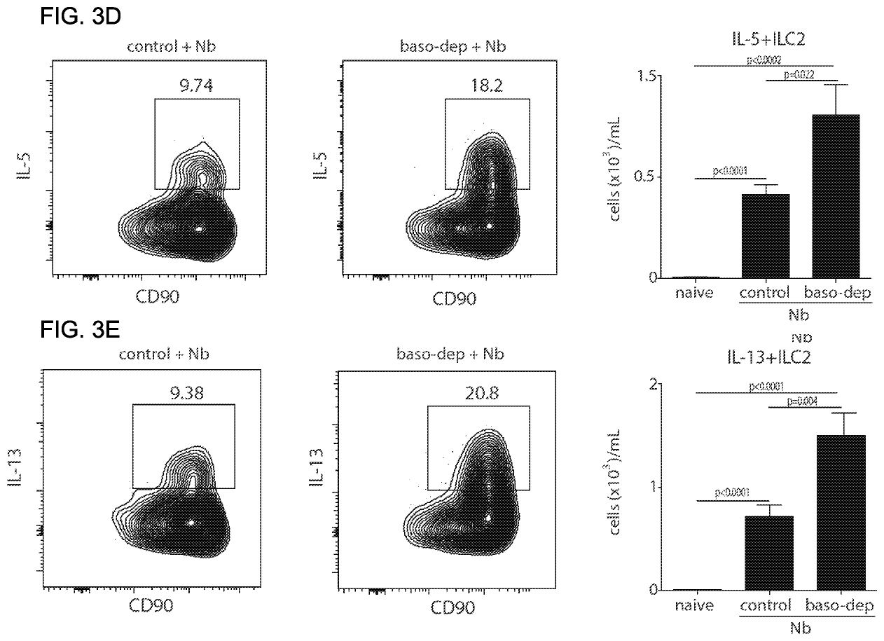

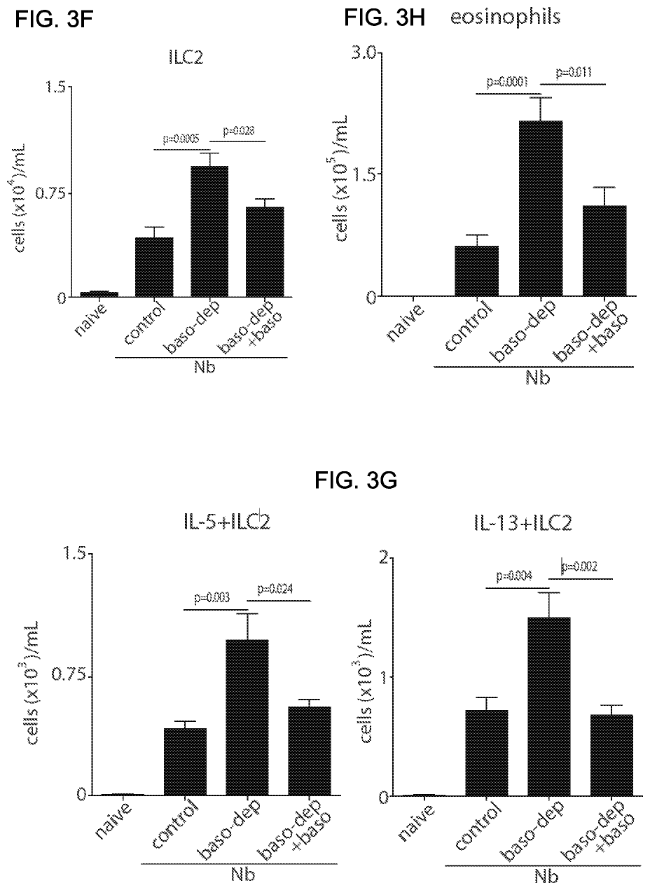

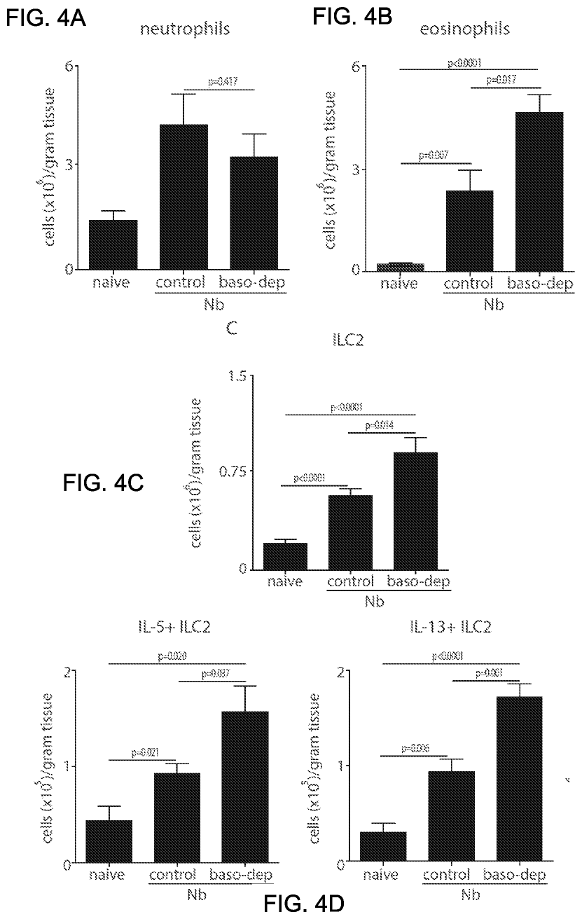

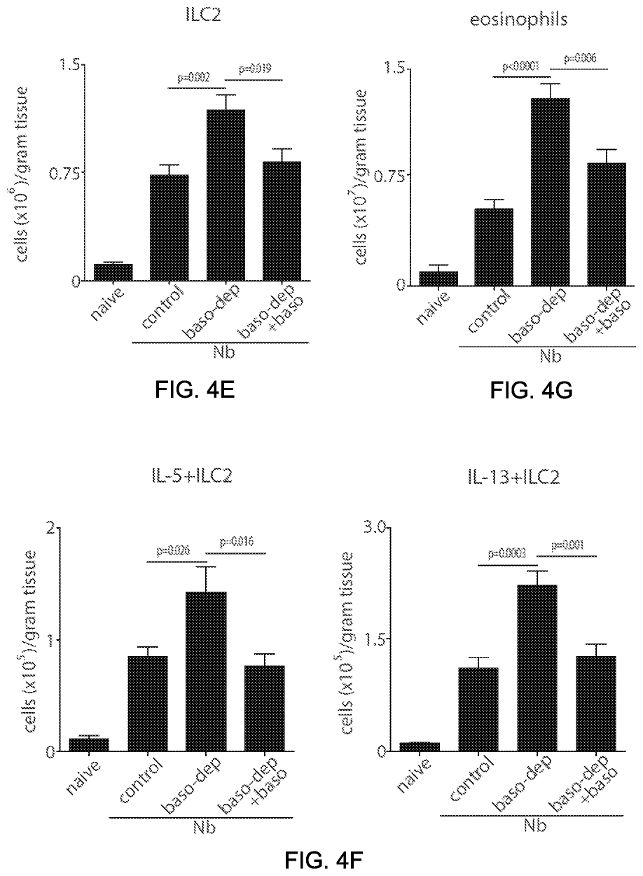

Flow cytometric analysis of bronchoalveolar lavage (BAL) fluid and lung infiltrates confirmed that while infection-induced neutrophilia was not significantly altered (FIGS. 3A, 4A), basophil-depleted mice exhibited significantly increased BAL and lung eosinophil responses (FIGS. 3B, 4B). Nb-induced eosinophil responses and mucus production are dependent on IL-5 and IL-13 that is produced by type 2 innate lymphoid cells (ILCS2s) and/or CD4+ T cells (1-3). Lung basophil responses are occurring in the first few days post-infection (day 3-5) during the innate window when ILC2 responses are important (13, 14). Further, the ability of basophils to communicate with and alter the activation state of ILC2s has been demonstrated (2).

Therefore, it was determined whether basophil-depleted animals exhibited elevated infection-induced ILC2 responses that correlated with increased mucus production and eosinophilia (14). Interestingly, ILC2s populations were increased in both the BAL and lung tissue of basophil-depleted mice compared to controls (FIGS. 3C, 4C). Further, increases in IL-5 and IL-13 producing ILC2s were also detected in BAL and lung tissue of basophil-depleted mice post-infection (FIGS. 3D, 3E, 4D). To rule out the possibility of off-target depletion effects, a gain-of-function approach was used. Diphtheria toxin receptor (DTR) negative basophils were introduced into basophil-depleted mice. The intratracheal transfer of DTR negative basophils into basophil-depleted mice was sufficient to suppress ILC2 responses and eosinophilia back to WT levels in both the BAL and lung (FIGS. 3F-3H, 4E-4G). Collectively, these loss- and gain-of-function approaches indicate that basophils negatively regulate lung ILC2s responses following Nb infection.

### Example 4

### Basophils Promote Expression of Nmbr on ILC2s

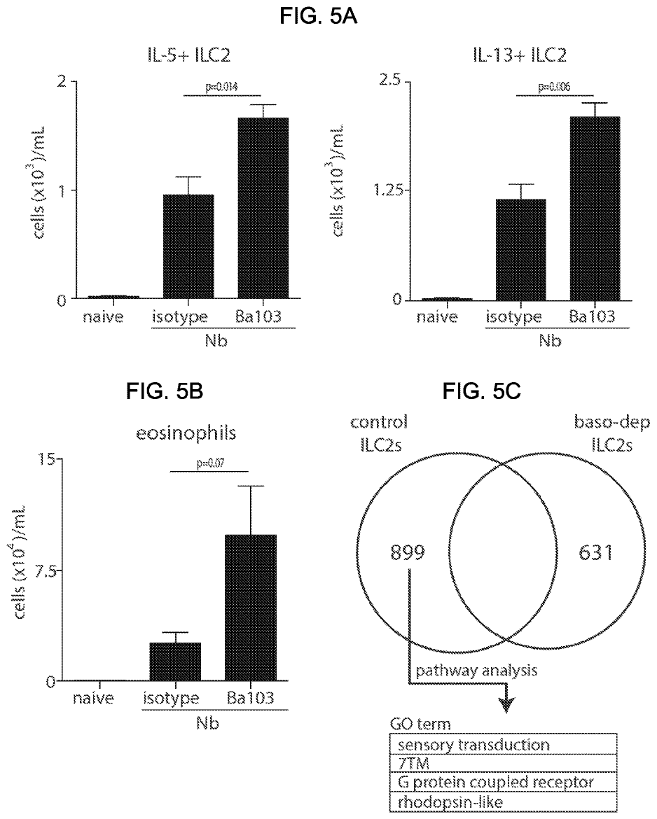

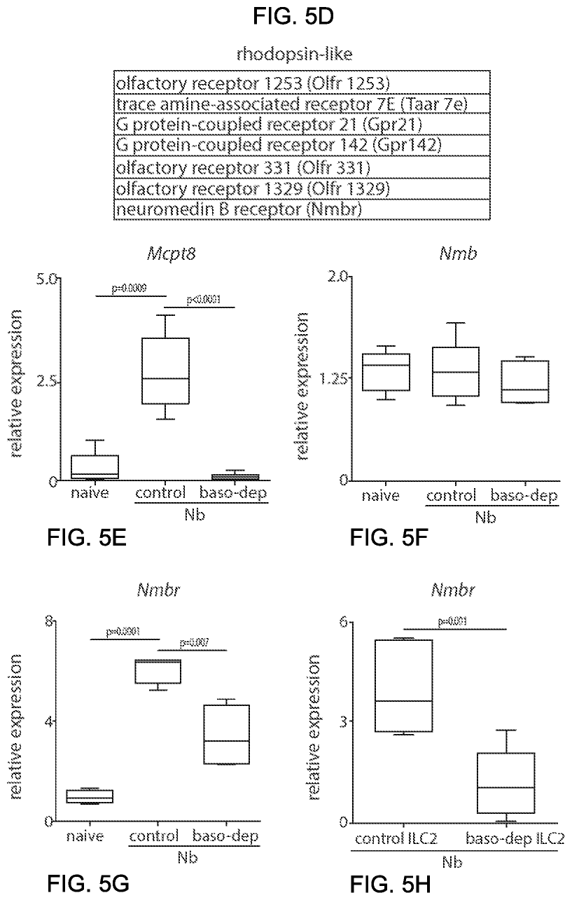

To confirm that the effects of basophil depletion were occurring independently of adaptive lymphocytes, recombination-activation gene (Rag2)-deficient mice were treated with the basophil-depleting antibody Ba103. Rag2−/− mice treated with Ba103 exhibited significantly elevated Nb-induced ILC2 responses (FIG. 5A) and elevated eosinophilia (FIG. 5B) compared to control mice. Collectively, both loss- and gain-of-function approaches indicate that basophils negatively regulate lung ILC2s responses following Nb infection.

To identify the mechanism through which basophils are regulating ILC2s, genome-wide transcriptional profiling was performed on lung ILC2s post-Nb. ILC2s from control animals were enriched for pathways associated with sensory transduction, seven-pass-transmembrane domain receptor (7TM), G-protein-coupled receptor signaling and rhodopsin-like signaling compared to ILC2s sort-purified from basophil-depleted mice (FIG. 5C).

Remarkably, one of the driving genes associated with these pathways was the neuromedin B receptor (Nmbr) (FIG. 5D). Neuromedin B (Nmb) is part of the neuromedin family of peptides including neuromedin A, B, C, K, L, N, S and U (15, 16). Neuromedin B is a bombesin-like peptide that is expressed in the central nervous system, lungs, gastrointestinal track and adipose tissues of mammals (15, 17). Previous studies suggest that Nmb and its receptor are localized to neurons found in the submucosa (18). Upon binding to its receptor, Nmb is reported to regulate cell growth, body temperature, blood pressure and glucose levels, however, its ability to regulate immunity and inflammation remains to be defined (15).

To investigate whether basophils regulate Nmb signaling pathways in the lung following Nb infection, expression of Nmb and its receptor in the presence or absence of basophils (as confirmed by reduced Mcpt8 expression), was examined (FIG. 5E). No changes in Nmb expression were detected following Nb infection (FIG. 5F); however, Nmbr was expressed at significantly higher levels in infected animals. Further, basophil depletion resulted in significantly reduced Nmbr expression in the lung (FIG. 5G). Consistent with RNAseq analysis, Nmbr expression was also significantly reduced in ILC2s sort-purified from basophil-deficient mice compared to controls (FIG. 5H).

### Example 5

### Nmb Suppresses Type 2 Cytokine Responses

To determine if Nmb operates as a negative regulator of type 2 cytokine responses, mice were infected with Nippostrongylus and treated with recombinant native Nmb (SEQ ID NO: 1). On day 7 post-infection parameters of type 2 cytokine-dependent inflammation were evaluated.

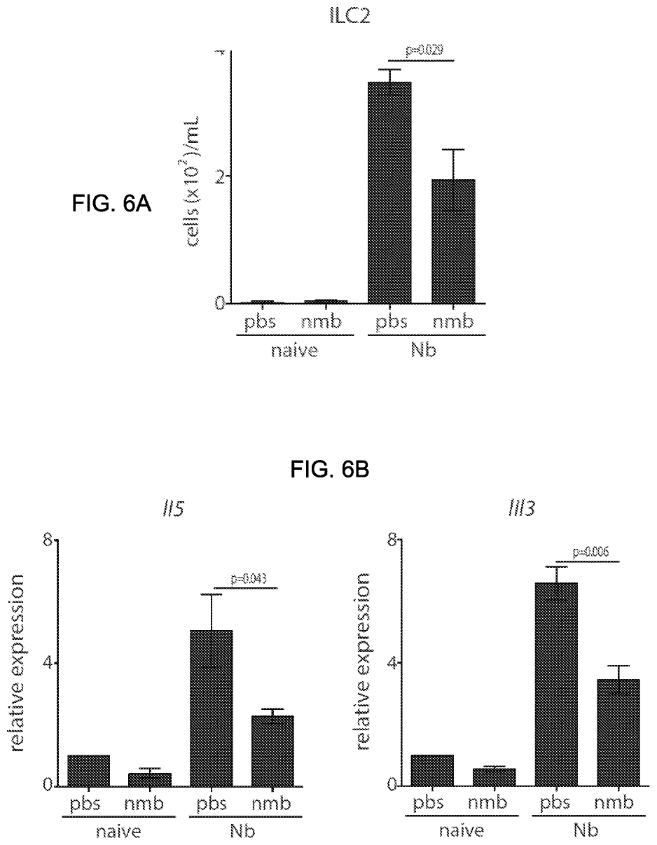

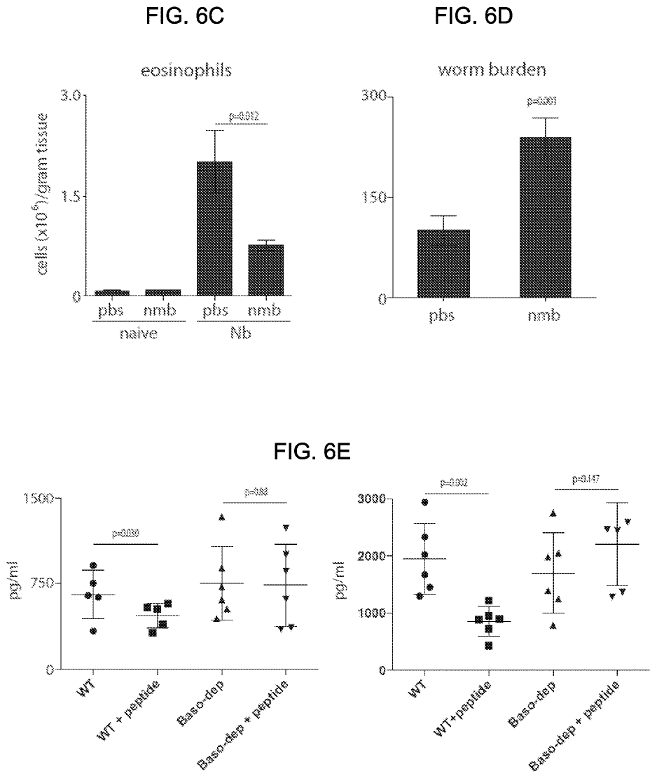

Strikingly, while Nmb treatment had no effect on infection-induced neutrophilia, Nmb-treated mice exhibited reduced ILC2 responses (FIG. 6A), IL-5 and IL-13 expression (FIG. 6B), eosinophilia (FIG. 6C) and failed to clear worms as efficiently as control mice (FIG. 6D). These data indicate that Nmb operates as a negative regulator of type 2 cytokine-mediated immunity.

To determine if Nmb can directly inhibit Nb-activated ILC2s in the lung, and whether basophils regulate this process, lung ILC2s from Nb-infected control and basophil-depleted mice were sort-purified and cultured overnight in the presence or absence of Nmb. Remarkably, treatment with Nmb significantly reduced IL-5 and IL-13 production by ILC2s isolated from basophil-sufficient, but not basophil-deficient mice (FIG. 6E).

### Example 6

### Nmb Treatment is Sufficient to Suppress Allergic Airway Inflammation

ILC2 responses are major contributors to the type 2 cytokine production, eosinophilia, and mucus production associated with allergic inflammation (14). Further, activated ILC2s play roles in activating TH2 cells that are critical regulators of allergic inflammation (13, 14). Therefore, ILC2s represent a cell population of significant therapeutic potential that can be targeted to treat multiple forms of allergic disease. The data presented above indicate that Nmb can inhibit ILC2 responses in the context of a parasitic helminth infection. To demonstrate that Nmb can be employed to treat allergic inflammation the following experiments were performed.

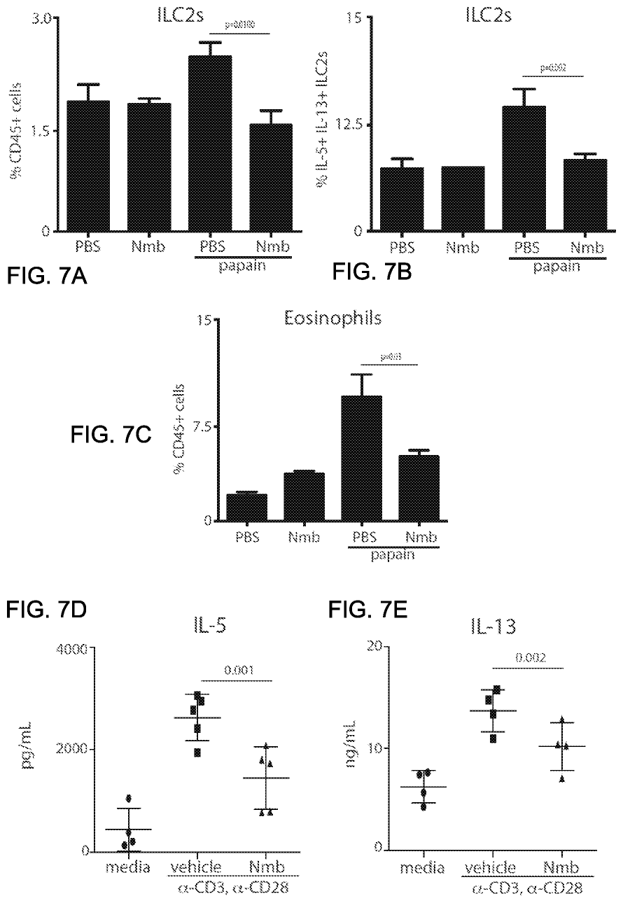

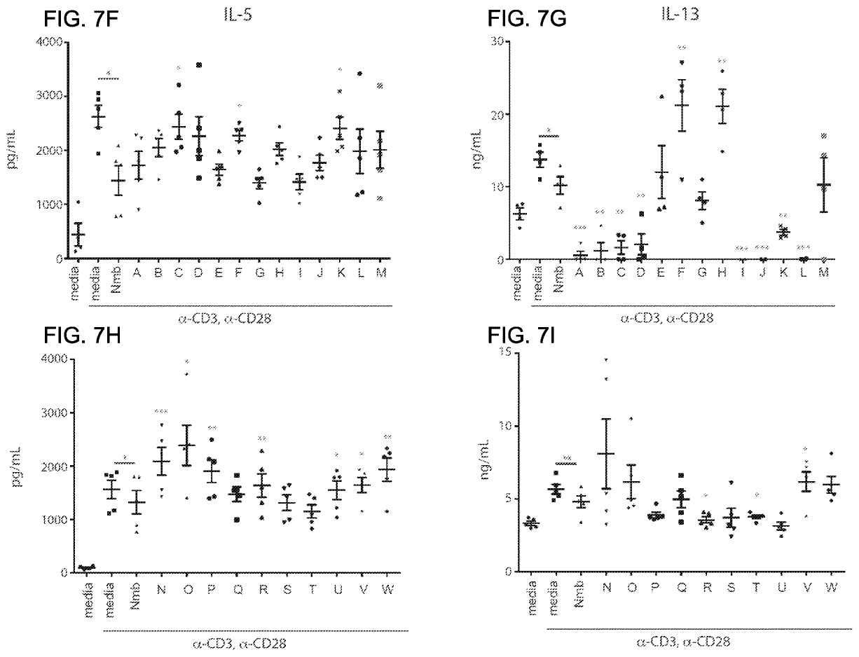

A well-established model of papain-induced allergic airway inflammation known to be dependent on ILC2s was utilized (19, 20). Mice treated with papain presented with elevated percentages of ILC2s (FIG. 7A), elevated expression of IL-5 and IL-13 by ILC2s (FIG. 7B) and lung eosinophilia (FIG. 7C). Critically, in vivo administration of Nmb was sufficient to reduce papain-induced ILC2 responses and eosinophilia (FIG. 7A-7C), demonstrating that Nmb can be employed therapeutically to treat allergic inflammation. Thus, Nmb negatively regulate ILC2 responses both in vivo and in vitro.

ILC2s share many common characteristics with their adaptive TH2 counterparts, therefore, it is possible that Nmb also possesses the ability to inhibit TH2 cells (13, 14). At the time points evaluated post-Nb, the majority of the type 2 cytokines are ILC2-derived (14) and any effects Nmb is having on TH2 cell responses may be difficult to detect. To address this, lung-draining LNs were isolated on day 7 post-Nb and stimulated with anti-CD3 and -CD28 to activate T cells in both the presence of absence of recombinant Nmb. LN cells treated with anti-CD3 and -CD28 produced increased amounts of IL-5, and IL-13 (FIGS. 7D, 7E). Cultures treated with Nmb exhibited significantly reduced levels of IL-5 and 13 (FIGS. 7D, 7E). These data demonstrate that Nmb also inhibits the production of cytokines from activated T_{H}2 cells.

### Example 7

### Nmb Protein Variants

The results above demonstrate that Nmb operates as a negative regulator of both ILC2s and TH2 cell responses and is sufficient to reduce type 2 cytokine-mediated inflammation, for example in a subject having a parasitic infection and or allergic airway inflammation. Thus, Nmb can be employed to reduce type 2 cytokine-mediated inflammation. Native Nmb is a 10-mer peptide (SEQ ID NO: 1) that operates via its ability to bind the Nmb receptor (15). This example describes the results of an alanine scan to systematically replace various residues of the Nmb with alanine (Table 2) and the resulting ability of the variant Nmb peptides to inhibit type 2 cytokine production by TH2 cells. Further, caps were added to the native form of Nmb (SEQ ID NO: 1) and its bioactivity monitored (FIG. 5A).

TABLE 2
Variant Nmb peptides

| Code | Compound Name | Structure (SEQ ID NO:) |
|---|---|---|
| NMB | WT Neuromedin B | H2N-GNLWATGHFM-amide (1) |
| Compound A | Alanine Scan Peptide 1 | H2N-ANLWATGHFM-amide (3) |
| Compound B | Alanine Scan Peptide 2 | H2N-GALWATGHFM-amide (4) |
| Compound C | Alanine Scan Peptide 3 | H2N-GNAWATGHFM-amide (5) |
| Compound D | Alanine Scan Peptide 4 | H2N-GNLAATGHFM-amide (6) |
| Compound E | Alanine Scan Peptide 5 | H2N-GNLWAAGHFM-amide (7) |
| Compound F | Alanine Scan Peptide 6 | H2N-GNLWATAHFM-amide (8) |
| Compound G | Alanine Scan Peptide 7 | H2N-GNLWATGAFM-amide (9) |
| Compound H | Alanine Scan Peptide 8 | H2N-GNLWATGHAM-amide (10) |
| Compound I | Alanine Scan Peptide 9 | H2N-GNLWATGHFA-amide (11) |
| Compound J | Different Caps Peptide 1 | Ac-GLLWATGHFM-amide (12) |
| Compound K | Different Caps Peptide 2 | Methanesulfonyl-GNLWATGHFM-amide |
| | | (13) |
| Compound L | Different Caps Peptide 3 | H2N-GNLWATGHFM-NHMe (14) |
| Compound M | Free Acid | H2N-GNLWATGHFM-OH (15) |
| Compound N | Truncations peptide 1 | H2N-NLWATGHFM-amide (16) |
| Compound O | Truncations peptide 2 | H2N-GLWATGHFM-amide (17) |
| Compound P | Truncations peptide 3 | H2N-GNWATGHFM-amide (18) |
| Compound Q | Truncations peptide 4 | H2N-GNLATGHFM-amide (19) |
| Compound R | Truncations peptide 5 | H2N-GNLWTGHFM-amide (20) |
| Compound S | Truncations peptide 6 | H2N-GNLWAGHFM-amide (21) |
| Compound T | Truncations peptide 7 | H2N-GNLWATHFM-amide (22) |
| Compound U | Truncations peptide 8 | H2N-GNLWATGFM-amide (23) |
| Compound V | Truncations peptide 9 | H2N-GNLWATGHM-amide (24) |
| Compound W | Truncations peptide 10 | H2N-GNLWATGHF-amide (25) |
| Compound X | Length 8 Offset 1 Peptide 1 | H2N-GNLWATGH-amide (26) |
| Compound Y | Length 8 Offset 1 Peptide 2 | H2N-NLWATGHF-amide (27) |
| Compound Z | Length 8 Offset 1 Peptide 3 | H2N-LWATGHFM-amide (28) |
| Compound AA | Length 7 Offset 1 Peptide 1 | H2N-GNLWATG-amide (29) |
| Compound AB | Length 7 Offset 1 Peptide 2 | H2N-NLWATGH-amide (30) |
| Compound AC | Length 7 Offset 1 Peptide 3 | H2N-LWATGHF-amide (31) |
| Compound AD | Length 7 Offset 1 Peptide 4 | H2N-WATGHFM-amide (32) |
| Compound AE | Length 6 Offset 1 Peptide 1 | H2N-GNLWAT-amide (33) |
| Compound AF | Length 6 Offset 1 Peptide 2 | H2N-NLWATG-amide (34) |
| Compound AG | Length 6 Offset 1 Peptide 3 | H2N-LWATGH-amide (35) |
| Compound AH | Length 6 Offset 1 Peptide 4 | H2N-WATGHF-amide (36) |
| Compound AI | Length 6 Offset 1 Peptide 5 | H2N-ATGHFM-amide (37) |

While peptides C, F and K (SEQ ID NOS: 5, 8 and 13, respectively) exhibited a significantly reduced capacity to inhibit IL-5 production compared to the native form of Nmb (SEQ ID NO: 1), none of the Nmb variant peptides showed a significantly enhanced capacity to inhibit IL-5 production (FIG. 7F). In contrast, peptides F and H (SEQ ID NOS: 8 and 10, respectively) had a significantly reduced capacity to inhibit IL-13 production and peptides A, B, C, D, I, J, K and L (SEQ ID NOS: 3, 4, 5, 6, 11, 12, 13, and 14, respectively) had a significantly greater capacity to inhibit IL-13 compared to native Nmb (FIG. 7G).

The ability of truncated versions of Nmb (Table 2, SEQ ID NOS: 16-25) to regulate type 2 cytokine production from T_{H}2 cells was examined. Native Nmb was sufficient to significantly reduce IL-5 production from T_{H}2 cells, however, truncated peptides N, O, P, R, U, V and W (SEQ ID NOS: 16, 17, 18, 20, 23, 24 and 25, respectively) showed a reduced capacity to suppress IL-5 production (FIG. 7H). Interestingly, while truncated peptide V (SEQ ID NO: 24) showed a reduced capacity to inhibit IL-13, peptides R and T (SEQ ID NOS: 20 and 22, respectively) showed an enhanced capacity to inhibit IL-13 from T_{H}2 cells (FIG. 7I).

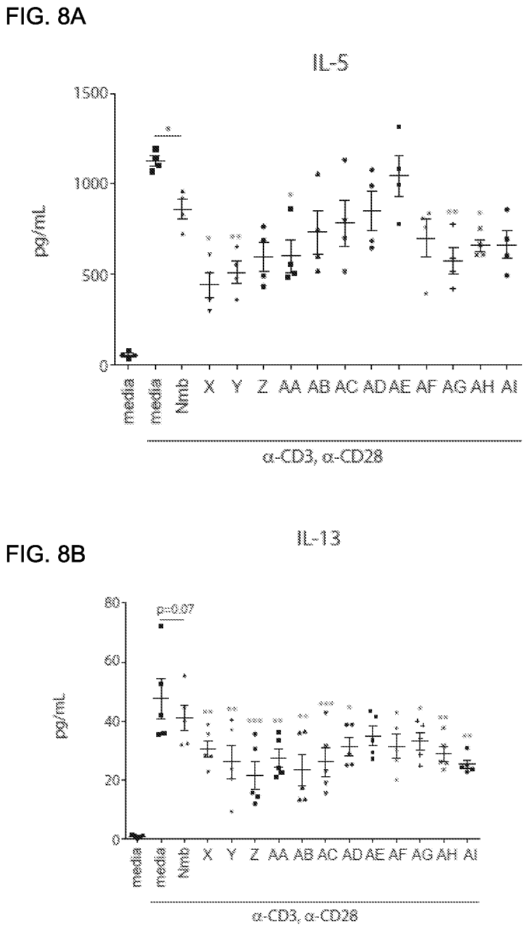

The effect of offset lengths (Table 2, SEQ ID NOS: 26-36) on altered capacities to regulate type 2 cytokine production from T_{H}2 cells was examined. As previously shown above, native Nmb was sufficient to reduce IL-5 and IL-13 production from T_{H}2 cells. Offset peptides X (SEQ ID NO: 26), Y (SEQ ID NO: 27), AA (SEQ ID NO: 29), AG (SEQ ID NO: 35), and AH (SEQ ID NO: 36), had significant enhanced capacity to suppress IL-5 production from activated T_{H}2 cells (FIG. 8A). In addition, peptides X (SEQ ID NO: 26), Y (SEQ ID NO: 27), Z (SEQ ID NO: 28), AA (SEQ ID NO: 29), AB (SEQ ID NO: 30), AC (SEQ ID NO: 31), AD (SEQ ID NO: 32), AG (SEQ ID NO: 35), AH (SEQ ID NO: 36) and AI (SEQ ID NO: 37) showed and an enhanced capacity to reduce IL-13 production from T_{H}2 cells (FIG. 8B).

Collectively, these data indicate that Nmb can be modified to inhibit type 2 cytokine production and these altered peptides may possess greater therapeutic potential to modify type 2 inflammation than the native version of Nmb (SEQ ID NO: 1).

In addition, these data indicate combinations of Nmb variant peptides can be used in the disclosed methods, such as a combination of at least one variant peptide that reduces IL-5, and at least one variant peptide that reduces IL-13, such as combinations of peptides shown in Table 3. Furthermore, In addition, these data indicate native Nmb (SEQ ID NO: 1) can be used in combination with one or more Nmb variant peptides, such as a combination of (1) native Nmb with at least one variant peptide that reduces IL-5, (2) native Nmb with and at least one variant peptide that reduces IL-13, or (3) native Nmb with at least one variant peptide that reduces IL-5 and at least one variant peptide that reduces IL-13 (such as peptides shown in Table 3). In one example, the method uses at least one Nmb variant peptide with an enhanced ability to decrease IL-5, such as one or more of peptides X (SEQ ID NO: 26), Y (SEQ ID NO: 27), AA (SEQ ID NO: 29), AG (SEQ ID NO: 35), and AH (SEQ ID NO: 36). In one example, the method uses at least one Nmb variant peptide with an enhanced ability to decrease IL-13, such as one or more of peptides A, B, C, D, I, J, K, L, R, T, X, Y, Z, AA, AB, AC, AD, AG, AH, and AI (SEQ ID NOS: 3, 4, 5, 6, 11, 12, 13, 14, 20, 22, 26, 27, 28, 29, 30, 31, 32, 35, 36 and 37, respectively).

TABLE 3
Variant Nmb Peptides that reduced IL-5 or IL-13

| Peptides that reduce IL-5 | Peptides that reduce IL-13 |
|---|---|
| Peptides A, B, D, E, G, H, I, J, L, M, Q, S, T, X, Y, | Peptides A, B, C, D, G, I, J, K, L, P, Q, R, |
| Z, AA, AB, AD, AF, AG, AH, and AI (SEQ ID | S, T, U (SEQ ID NOs: 3, 4, 5, 6, 9, 11, |
| NOs: 3, 4, 6, 7, 9, 10, 11, 12, 14, 15, 19, 21, 22, 26, | 12, 13, 14, 18, 19, 20, 21, 22, 23) |
| 27, 28, 29, 30, 32, 34, 35, 36, 37) | |

### Example 8

### Neuromedin C Protein

In some examples, instead of (or in addition to) using an Nmb protein (or variant thereof) in the disclosed methods and compositions, a neuromedin C protein (or variant thereof) is used. An exemplary native neuromedin C protein is GNHWAVGHLM (SEQ ID NO: 38). Alternatively, a nucleic acid molecule encoding neuromedin C is used. Thus, a protein having at least at least 50%, at least 60%, at least 70%, at least 80%, at least 90%, or 100% sequence identity to SEQ ID NO: 38, or a one nucleic acid molecule encodes at least one neuromedin C protein having at least 50%, at least 60%, at least 70%, at least 80%, at least 90%, or 100% sequence identity to SEQ ID NO: 38, can be used in the disclosed methods. Previous studies in mammals have suggested that Nmb and Nme are capable of interacting with a common receptor (PMID 1720612). Therefore, Nme and Nmb may initiate common signaling pathways that are capable of inhibiting type 2 inflammation.

### Example 9

### Basophils Regulate Nmbr Expression

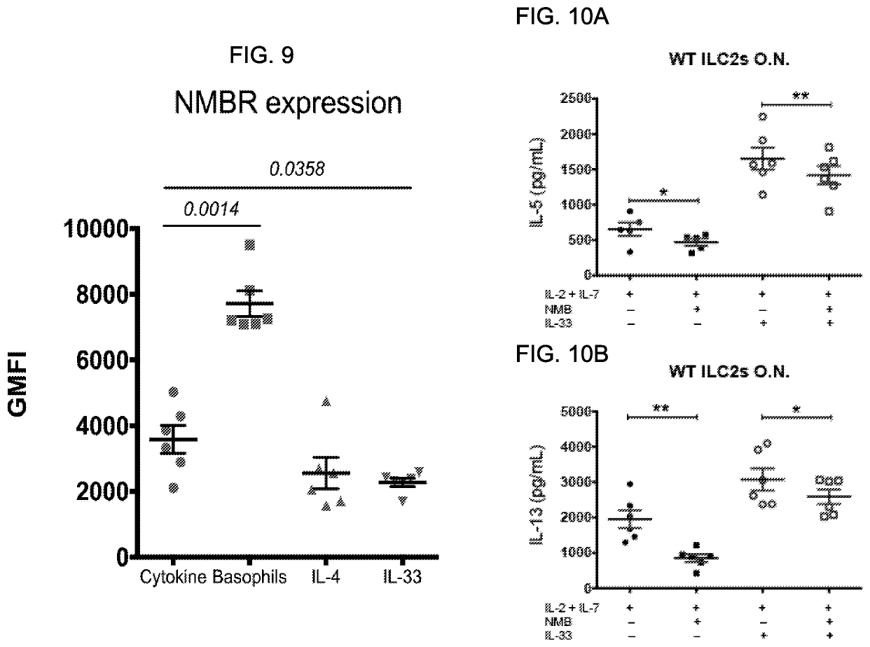

NMBR expression by lung ILC2s was reduced when basophils are depleted following an Nb infection. To determine whether basophils directly mediate expression of NMBR or whether they operate through intermediate pathways, ILC2s were sort-purified from the lungs of Nb-infected mice (day 7) and cultured with the survival cytokines IL-2 and IL-7. Additionally, the ILC2s were cultured with activated basophils, IL-4, or IL-33 overnight. NMBR expression was subsequently monitored by flow cytometric analysis post-culture. The resulting data indicates that while ILC2s treated with IL-4 or IL-33, cytokines known to promote ILC2 activation (21, 23, 24), showed no changes in NMBR expression levels, ILC2s cultured with activated basophils showed significantly increased expression of NMBR (FIG. 9). Collectively, these results demonstrate that activated basophils are sufficient to directly regulate NMBR expression by ILC2s.

These studies illustrate that Nb-induced ILC2 responses are exaggerated in the absence of basophils. Further, the data suggest that reduced NMB-NMBR signaling in the absence of basophils may result in a heightened state of ILC2 activation. However, ILC2 responses are also known to be regulated by several other factors following a Nb challenge. Specifically, IL-33 is reported to be a critical regulator of lung ILC2 responses post-Nb infection (24). To address whether altered ILC2 activity in the absence of basophils is a result of reduced availability of IL-33, rather than alterations in NMB-NMBR signaling pathways, ILC2s were sort-purified from the lungs of Nb-infected mice (day 7) and cultured with survival cytokines (IL-2, IL-7) and/or IL-33 and NMB. Following overnight culture, supernatants were tested for the presence of IL-5 and IL-13 by standard ELISA. Statistical comparisons were done using a Student's t-test. The data indicates that NMB inhibits type 2 cytokine production from activated ILC2. In particular, NMB was sufficient to significantly reduce IL-5 and IL-13 production from ILC2s at steady-state (FIGS. 10A-10B). Further, these data reveal that while IL-33 treatment resulted in increased levels of IL-5 and IL-13, NMB treatment still resulted in significant reductions in type 2 cytokines. Taken together, these studies demonstrate that NMB operates as a negative regulator of ILC2s even when robust amounts of IL-33 are present.

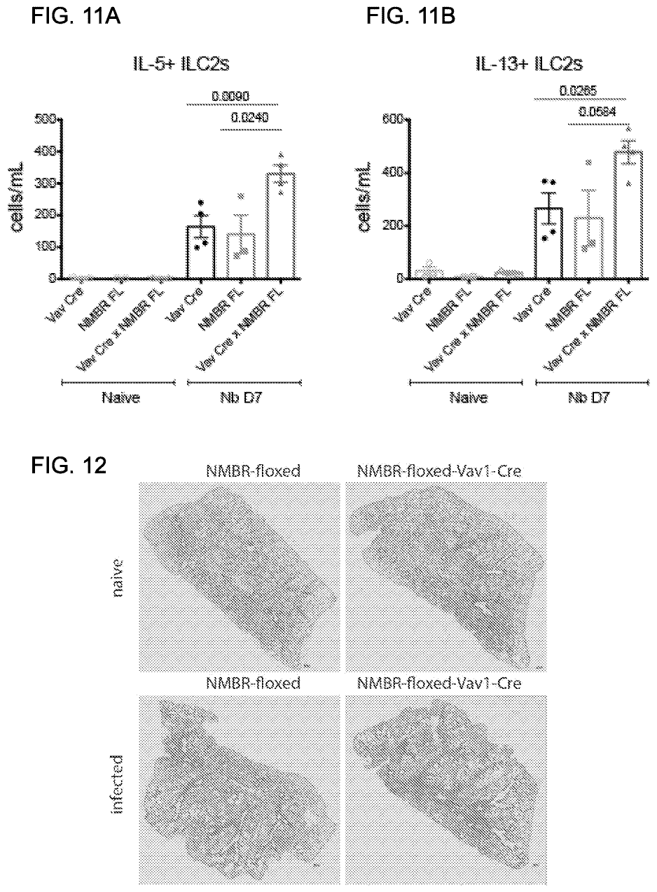

The disclosed studies suggest that NMB-NMBR signaling on hematopoietic cells is required to properly regulate type 2 cytokine responses. To further evaluate this possibility, novel NMBR-floxed mice were generated and crossed to Vav1-Cre expressing mice. Vav1 is expressed by all hematopoietic cells (22) and crossing them to the novel floxed mouse model resulted in the selective deletion of NMBR on all immune cells. Next, these mice were infected with Nb and type 2 cytokine responses and lung pathology were evaluated. Intracellular staining for IL-5 (FIG. 11A) and IL-13 (FIG. 11B) was performed on ILC2s isolated from the BAL of naive or Nb-infected (day 7), Vav1-Cre, NMBR-floxed, or Vav1-Cre-NMBR-floxed mice. Statistical comparisons were done using a Student's t-test. The data indicate that NMBR-mediated signaling on hematopoietic cells is required to regulate type 2 cytokine production. Consistent with previous data, genetic deletion of NMBR on immune cells resulted in significantly elevated IL-5 and IL-13 production by ILC2s isolated for the BAL of infected animals (FIGS. 11A-11B). Lung pathology (H and E staining) was evaluated for naive or Nb-infected (day 7), Vav1-Cre, NMBR-floxed, or Vav1-Cre-NMBRfloxed mice. NMBR-floxed-Vav1-Cre mice presented with increased cellular infiltrates in the lung and markedly worse lung pathology (FIG. 12). This indicates that NMBR-mediated signaling on hematopoietic cells is required to regulate (e.g., decrease) cellular infiltrates in the lung. These genetic approaches further confirm the importance of NMB-NMBR signaling on immune cells in properly regulating type 2 cytokine-mediated inflammation.

The data presented above suggest that NMB is an important negative regulator of type 2 cytokine responses by lymphocytes. To gain further insight into how NMB might alter lymphocyte activation, ILC2s were sort-purified from the lungs of Nb-infected mice and cultured overnight with survival cytokines (IL-2, IL-7), with or without Nmb. Then, gene expression was evaluated by RNA sequencing analysis. Specifically, following culture, cells were submitted for RNA sequencing and genes that were significantly upregulated or down regulated (>1.5 fold) by NMB treatment were identified (Table 4). This allowed for identification of NMB signaling pathways. The results indicate that NMB treatment resulted in the upregulation of several genes including Sprr2a2, Serpinb2, Il1b, Xist and Tsix among others. Further, NMB treatment resulted in the downregulation of Hgs2, Nkg7, Klra7, P2rx7, Lγδc2 and Mcpt2 among others. Collectively, these data suggest that NMB may operate by influencing the genes listed in Table 4 and that these genes may be of substantial therapeutic potential.

TABLE 4
NMB signaling pathways

| Name | Chromosome | Region | Fold change | FDR p-value | ENSEMBL |
|---|---|---|---|---|---|
| Igkv17-127 | 6 | 67861153 . . . 67861659 | 800.9031394 | 1.39349E−09 | ENSMUSG00000076508 |
| Ighg2b | 12 | complement(113302965 . . . 113307933) | 276.0317888 | 1.29827E−08 | ENSMUSG00000076613 |
| Ighg1 | 12 | complement(113325240 . . . 113330523) | 253.5888585 | 7.33518E−10 | ENSMUSG00000076614 |
| Igkv1-135 | 6 | 67609713 . . . 67610508 | 47.49234298 | 0.049956905 | ENSMUSG00000096336 |
| Igkv8-28 | 6 | complement(70143593 . . . 70144166) | 43.80053615 | 0.038814357 | ENSMUSG00000094356 |
| Sprr2a2 | 3 | 92215920 . . . 92256724 | 37.85216327 | 0.000515772 | ENSMUSG00000068893 |
| Serpinb2 | 1 | 107511423 . . . 107535478 | 5.695871502 | 0.002291872 | ENSMUSG00000062345 |
| Il1b | 2 | complement(129364570 . . . 129371139) | 3.263058363 | 0.005266113 | ENSMUSG00000027398 |
| Xist | X | complement(103460375 . . . 103483217) | 3.076866923 | 1.37162E−10 | ENSMUSG00000086503 |
| Tsix | X | 103431517 . . . 103484957 | 2.833195718 | 1.45251E−05 | ENSMUSG00000085715 |
| Hgs_2 | 11 | 120467635 . . . 120483984 | −1.89340553 | 0.043638034 | ENSMUSG00000116045 |
| Nkg7 | 7 | 43437073 . . . 43438249 | −1.894603182 | 0.000515772 | ENSMUSG00000004612 |
| Klra7 | 6 | complement(130218605 . . . 130233322) | −1.984286795 | 0.038814357 | ENSMUSG00000067599 |
| P2rx7 | 5 | 122643911 . . . 122691432 | −2.148918914 | 0.002437103 | ENSMUSG00000029468 |
| Ly6c2 | 15 | complement(75108158 . . . 75111970) | −2.368890009 | 0.001898784 | ENSMUSG00000022584 |
| Gm10031 | 1 | 156524012 . . . 156526664 | −6.202778267 | 0.000138883 | ENSMUSG00000101523 |
| Mcpt2 | 14 | 56042041 . . . 56044642 | −11.85721712 | 0.005266113 | ENSMUSG00000022226 |
| Gm14288 | 2 | complement(175789357 . . . 175801093) | −25.4323313 | 0.009218816 | ENSMUSG00000078889 |

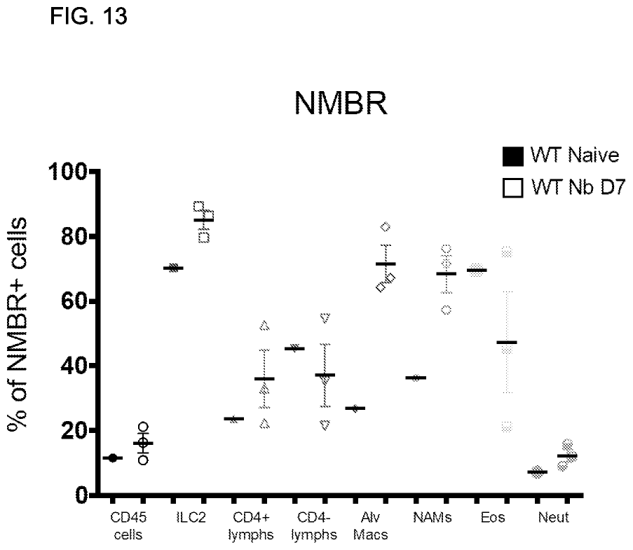

The mouse model discussed above demonstrated that NMBR expression on CD45+ cells is required to properly regulate type 2 cytokine-mediated inflammation (FIGS. 11A-B, 12). To better determine the cell types that may require this signaling pathway to be properly regulated expression of NMBR on immune cells in the lung was evaluated at steady-state and on day 7 post-Nb. The data indicates that NMBR is expressed by several immune cell populations. ILC2s, CD4+ and CD4-lymphocytes, Alveolar macrophages, Non alveolar macrophages, eosinophils and neutrophils were found to be expressing varying levels of NMBR (FIG. 13). These data suggest that NMB may regulate inflammation via diverse cellular targets.

### Example 10

### Prostaglandin E2 Upregulates Expression of NMBR on Lymphocytes

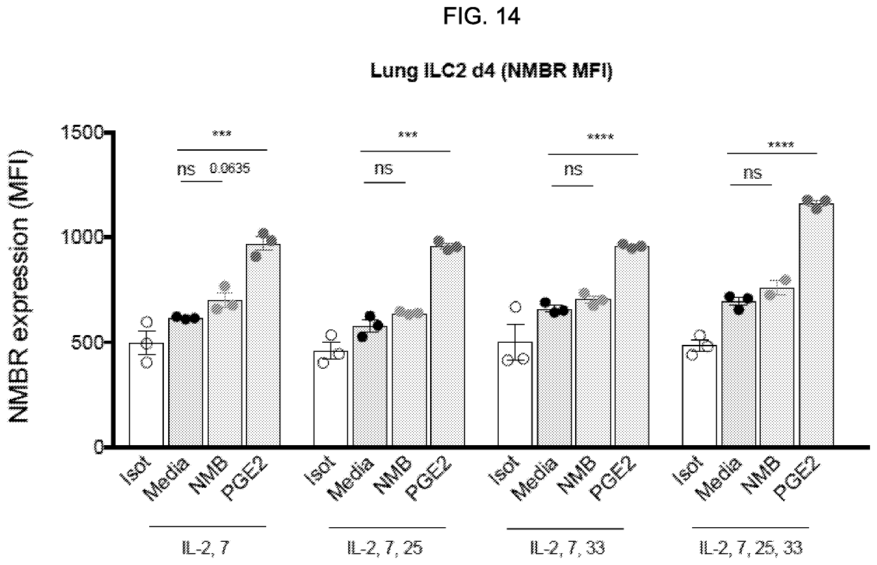

To better determine the mechanisms through which basophils regulate expression of NMBR on lymphocytes, ILC2s were cultured with a combination of survival and activating cytokines in the presence or absence of NMB or prostaglandin E2 (PGE2). While combinations of IL-25, IL-33, and NMB failed to upregulate NMBR expression by ILC2s, treatment with PGE2 resulted in significantly upregulated levels on NMBR (FIG. 14). These data suggest that basophils (well described sources of prostaglandins) may regulate NMBR expression through their release of PGE2.

In view of the data suggesting that PGE2 promotes expression of Nmbr by lymphocytes, an embodiment of a method of treatment of a disorder (such as an inflammatory disorder) may comprise administering to a subject an effective amount of a Nmb peptide or nucleic acid disclosed herein and an effective amount of PGE2.

### REFERENCES

1. Pulendran B, Artis D. New paradigms in type 2 immunity. Science. [Research Support, N.I.H., Extramural Research Support, Non-U.S. Gov't Review]. 2012 Jul. 27; 337(6093):431-5.
2. Henry E K, Inclan-Rico J M, Siracusa M C. Type 2 cytokine responses: regulating immunity to helminth parasites and allergic inflammation. Curr Pharmacol Rep. 2017 December; 3(6):346-59.
3. Wynn T A. Type 2 cytokines: mechanisms and therapeutic strategies. Nat Rev Immunol. [Research Support, N.I.H., Intramural Review]. 2015 May; 15(5):271-82.
4. Lloyd C M, Snelgrove R J. Type 2 immunity: Expanding our view. Sci Immunol. [Review]. 2018 Jul. 6; 3(25).
5. Rivera A, Siracusa M C, Yap G S, Gause W C. Innate cell communication kick-starts pathogen-specific immunity. Nat Immunol. [Research Support, N.I.H., Extramural Research Support, Non-U.S. Gov't Review]. 2016 April; 17(4):356-63.
6. Motomura Y, Morita H, Moro K, Nakae S, Artis D, Endo T A, et al. Basophil-derived interleukin-4 controls the function of natural helper cells, a member of ILC2s, in lung inflammation. Immunity. [Research Support, Non-U.S. Gov't]. 2014 May 15; 40(5):758-71.
7. Kim B S, Wang K, Siracusa M C, Saenz S A, Brestoff J R, Monticelli L A, et al. Basophils promote innate lymphoid cell responses in inflamed skin. J Immunol. [Clinical Trial Research Support, N.I.H., Extramural]. 2014 Oct. 1; 193(7):3717-25.
8. Klose C S N, Mahlakoiv T, Moeller J B, Rankin L C, Flamar A L, Kabata H, et al. The neuropeptide neuromedin U stimulates innate lymphoid cells and type 2 inflammation. Nature. [Research Support, N.I.H., Extramural Research Support, Non-U.S. Gov't Research Support, U.S. Gov't, Non-P.H.S.]. 2017 Sep. 14; 549(7671):282-6.
9. Cardoso V, Chesne J, Ribeiro H, Garcia-Cassani B, Carvalho T, Bouchery T, et al. Neuronal regulation of type 2 innate lymphoid cells via neuromedin U. Nature. [Research Support, Non-U.S. Gov't]. 2017 Sep. 14; 549(7671):277-81.
10. Kubo M. Mast cells and basophils in allergic inflammation. Curr Opin Immunol. [Review]. 2018 Jun. 28; 54:74-9.
11. Sullivan B M, Liang H E, Bando J K, Wu D, Cheng L E, McKerrow J K, et al. Genetic analysis of basophil function in vivo. Nat Immunol. 2011 June; 12(6):527-35.
12. Anderson K G, Mayer-Barber K, Sung H, Beura L, James B R, Taylor J J, et al. Intravascular staining for discrimination of vascular and tissue leukocytes. Nature protocols. [Research Support, N.I.H., Extramural
Research Support, N.I.H., Intramural
Research Support, Non-U.S. Gov't]. 2014 January; 9(1):209-22.
13. Colonna M. Innate Lymphoid Cells: Diversity, Plasticity, and Unique Functions in Immunity. Immunity. [Review]. 2018 Jun. 19; 48(6):1104-17.
14. Tait Wojno E D, Artis D. Emerging concepts and future challenges in innate lymphoid cell biology. J Exp Med. [Review Research Support, Non-U.S. Gov't Research Support, N.I.H., Extramural]. 2016 Oct. 17; 213(11):2229-48.
15. Ohki-Hamazaki H. Neuromedin B. Prog Neurobiol. [Review]. 2000 October; 62(3):297-312.
16. Gajjar S, Patel B M. Neuromedin: An insight into its types, receptors and therapeutic opportunities.

Pharmacol Rep. [Review]. 2017 June; 69(3):438-47.
17. Ohki-Hamazaki H, Iwabuchi M, Maekawa F. Development and function of bombesin-like peptides and their receptors. Int J Dev Biol. [Review]. 2005; 49(2-3):293-300.
18. Ma Z, Su J, Guo T, Jin M, Li X, Lei Z, et al. Neuromedin B and Its Receptor: Gene Cloning, Tissue Distribution and Expression Levels of the Reproductive Axis in Pigs. PLoS One. [Research Support, Non-U.S. Gov't]. 2016; 11(3):e0151871.
19. Halim T Y, Steer C A, Matha L, Gold M J, Martinez-Gonzalez I, McNagny K M, et al. Group 2 innate lymphoid cells are critical for the initiation of adaptive T helper 2 cell-mediated allergic lung inflammation. Immunity. [Research Support, Non-U.S. Gov't]. 2014 Mar. 20; 40(3):425-35.
20. Martinez-Gonzalez I, Matha L, Steer C A, Ghaedi M, Poon G F, Takei F. Allergen-Experienced Group 2 Innate Lymphoid Cells Acquire Memory-like Properties and Enhance Allergic Lung Inflammation. Immunity. [Research Support, Non-U.S. Gov't]. 2016 Jul. 19; 45(1):198-208.
21. Barlow, J. L., Bellosi, A., Hardman, C. S., Drynan, L. F., Wong, S. H., Cruickshank, J. P., and McKenzie, A. N. (2012). Innate IL-13-producing nuocytes arise during allergic lung inflammation and contribute to airways hyperreactivity. J Allergy Clin Immunol 129, 191-198.e191-194.
22. Joseph, C., Quach, J. M., Walkley, C. R., Lane, S. W., Lo Celso, C., and Purton, L. E. (2013). Deciphering hematopoietic stem cells in their niches: a critical appraisal of genetic models, lineage tracing, and imaging strategies. Cell stem cell 13, 520-533.
23. Motomura, Y., Morita, H., Moro, K., Nakae, S., Artis, D., Endo, T. A., Kuroki, Y., Ohara, O., Koyasu, S., and Kubo, M. (2014). Basophil-derived interleukin-4 controls the function of natural helper cells, a member of ILC2s, in lung inflammation. Immunity 40, 758-771.
24. Neill, D. R., Wong, S. H., Bellosi, A., Flynn, R. J., Daly, M., Langford, T. K., Bucks, C., Kane, C. M., Fallon, P. G., Pannell, R., et al. (2010). Nuocytes represent a new innate effector leukocyte that mediates type-2 immunity. Nature 464, 1367-1370.

In view of the many possible embodiments to which the principles of the disclosure may be applied, it should be recognized that the illustrated embodiments are only examples of the invention and should not be taken as limiting the scope of the invention. Rather, the scope of the invention is defined by the following claims. I therefore claim as my invention all that comes within the scope and spirit of these claims.

We claim:

1. A neuromedin B (Nmb) protein, comprising any of SEQ ID NOs: 3-37.

2. A composition comprising the Nmb protein of claim 1 and a pharmaceutically acceptable carrier or a liposome.

3. The Nmb protein of claim 1, further comprising a cell penetrating peptide, or an immunoglobulin Fc domain.

4. A nucleic acid encoding the Nmb protein of claim 1.

5. A method of treating an inflammatory disorder in a mammalian subject, comprising:
administering to the subject a therapeutically effective amount of the Nmb protein of claim 1, thereby treating the disorder.

6. A method of treating a disorder associated with undesirable interleukin-5 (IL-5) and/or IL-13 activity in a mammalian subject, comprising:
administering to the subject a therapeutically effective amount of the Nmb protein of claim 1, thereby treating the disorder.

7. The method of claim 6, wherein the Nmb protein comprises any of SEQ ID NO: 3-7, 9-15, 18-23, 26-30, 32, or 35-37.

8. The method of claim 5, wherein the inflammatory disorder is an allergy, an eosinophilic disorder, a disorder caused by overproduction of mucus, an airway disorder, a skin disorder, conjunctivitis, a parasitic infection, a fungal infection, or Hodgkin's lymphoma.

9. The method of claim 8, wherein the airway disorder is asthma, sinusitis, idiopathic pulmonary fibrosis (IPF), rhinitis, chronic obstructive pulmonary disease (COPD), or allergic airway inflammation.

10. The method of claim 8, wherein the skin disorder is eczema, atopic dermatitis, urticaria, chronic itch, or angioedema.

11. The method of claim 8, wherein the eosinophilic disorder is eosinophilic esophagitis, eosinophilic gastritis, eosinophilic gastroenteritis, eosinophilic enteritis, eosinophilic colitis, eosinophilic asthma, eosinophilic gastrointestinal disorder (EGID), eosinophilic fasciitis, eosinophilic granulomatosis with polyangiitis (EGPA), eosinophilic lung disorder, hypereosinophilic syndrome (HES), or eosinophilic leukemia.

12. A method of treating an inflammatory disorder in a mammalian subject, comprising:
administering to the subject a therapeutically effective amount of the nucleic acid of claim 4, thereby treating the disorder.

13. The method of claim 12, wherein the nucleic acid is part of a plasmid or viral vector.

14. The method of claim 5, wherein the administering comprises at least two separate administrations of the therapeutically effective amount of the Nmb protein.

15. The method of claim 5, further comprising administering to the subject a therapeutically effective amount of another therapeutic agent.

16. The method of claim 5, wherein the administering decreases inflammation, IL-5 activity, IL-13 activity, ILC2 responses, eosinophilia, mucus production, T cell responses, or any combination thereof.

17. The method of claim 5, wherein the Nmb protein comprises any of SEQ ID NOs: 3-37.

18. The method of claim 5, wherein the inflammatory disorder is IPF or COPD, and the administering decreases IL-13 activity.

19. The method of claim 5, further comprising administering to the subject a therapeutically effective amount of a neuromedin C (Nmc) protein, or a nucleic acid encoding the Nmc protein, thereby treating the disorder.

20. The method of claim 5, further comprising administering to the subject a therapeutically effective amount of prostaglandin E2 (PGE2).


SEQUENCE LISTING (58 sequences)

SEQ ID NO: 1 - protein, 10 aa, synthetic construct
  REGION 1..10: note = Neuromedin B with C-terminal amidation
  MOD_RES 10: note = AMIDATION
GNLWATGHFM

SEQ ID NO: 2 - DNA, 30 bp, Homo sapiens
  source 1..30: mol_type = other DNA
ggcaacctctgggccaccggtcacttcatg

SEQ ID NO: 3 - protein, 10 aa, synthetic construct
  REGION 1..10: note = Variant neuromedin B peptide
  MOD_RES 10: note = AMIDATION
ANLWATGHFM

SEQ ID NO: 4 - protein, 10 aa, synthetic construct
  REGION 1..10: note = Variant neuromedin B peptide
  MOD_RES 10: note = Amidation
GALWATGHFM

SEQ ID NO: 5 - protein, 10 aa, synthetic construct
  REGION 1..10: note = Variant neuromedin B peptide
  MOD_RES 10: note = AMIDATION
GNAWATGHFM

SEQ ID NO: 6 - protein, 10 aa, synthetic construct
  REGION 1..10: note = Variant neuromedin B peptide
  MOD_RES 10: note = AMIDATION
GNLAATGHFM

SEQ ID NO: 7 - protein, 10 aa, synthetic construct
  REGION 1..10: note = Variant neuromedin B peptide
  MOD_RES 10: note = AMIDATION
GNLWAAGHFM

SEQ ID NO: 8 - protein, 10 aa, synthetic construct
  REGION 1..10: note = Variant neuromedin B peptide
  MOD_RES 10: note = AMIDATION
GNLWATAHFM

SEQ ID NO: 9 - protein, 10 aa, synthetic construct
  REGION 1..10: note = Variant neuromedin B peptide
  MOD_RES 10: note = AMIDATION
GNLWATGAFM

SEQ ID NO: 10 - protein, 10 aa, synthetic construct
  REGION 1..10: note = Variant neuromedin B peptide
  MOD_RES 10: note = AMIDATION
GNLWATGHAM

SEQ ID NO: 11 - protein, 10 aa, synthetic construct
  REGION 1..10: note = Variant neuromedin B peptide
  MOD_RES 10: note = AMIDATION
GNLWATGHFA

SEQ ID NO: 12 - protein, 10 aa, synthetic construct
  REGION 1..10: note = Variant neuromedin B peptide
  SITE 1: note = Acetylation
  MOD_RES 10: note = Amidation
GLLWATGHFM

SEQ ID NO: 13 - protein, 10 aa, synthetic construct
  REGION 1..10: note = Variant neuromedin B peptide
  SITE 1: note = MISC_FEATURE - Methanesulfonyl
  MOD_RES 10: note = AMIDATION
GNLWATGHFM

SEQ ID NO: 14 - protein, 10 aa, synthetic construct
  REGION 1..10: note = Variant neuromedin B peptide
  SITE 10: note = MISC_FEATURE - N-methylation
GNLWATGHFM

SEQ ID NO: 15 - protein, 10 aa, synthetic construct
  REGION 1..10: note = Variant neuromedin B peptide
  MOD_RES 10: note = Free acid
GNLWATGHFM

SEQ ID NO: 16 - protein, 9 aa, synthetic construct
  REGION 1..9: note = Variant neuromedin B peptide
  MOD_RES 9: note = AMIDATION
NLWATGHFM

SEQ ID NO: 17 - protein, 9 aa, synthetic construct
  REGION 1..9: note = Variant neuromedin B peptide
  MOD_RES 9: note = AMIDATION
GLWATGHFM

SEQ ID NO: 18 - protein, 9 aa, synthetic construct
  REGION 1..9: note = Variant neuromedin B peptide
  MOD_RES 9: note = AMIDATION
GNWATGHFM

SEQ ID NO: 19 - protein, 9 aa, synthetic construct
  REGION 1..9: note = Variant neuromedin B peptide
  MOD_RES 9: note = AMIDATION
GNLATGHFM

SEQ ID NO: 20 - protein, 9 aa, synthetic construct
  REGION 1..9: note = Variant neuromedin B peptide
  MOD_RES 9: note = AMIDATION
GNLWTGHFM

SEQ ID NO: 21 - protein, 9 aa, synthetic construct
  REGION 1..9: note = Variant neuromedin B peptide
  MOD_RES 9: note = AMIDATION
GNLWAGHFM

SEQ ID NO: 22 - protein, 9 aa, synthetic construct
  REGION 1..9: note = Variant neuromedin B peptide
  MOD_RES 9: note = AMIDATION
GNLWATHFM

SEQ ID NO: 23 - protein, 9 aa, synthetic construct
  REGION 1..9: note = Variant neuromedin B peptide
  MOD_RES 9: note = AMIDATION
GNLWATGFM

SEQ ID NO: 24 - protein, 9 aa, synthetic construct
  REGION 1..9: note = Variant neuromedin B peptide
  MOD_RES 9: note = AMIDATION
GNLWATGHM

SEQ ID NO: 25 - protein, 9 aa, synthetic construct
  REGION 1..9: note = Variant neuromedin B peptide
  MOD_RES 9: note = AMIDATION
GNLWATGHF

SEQ ID NO: 26 - protein, 8 aa, synthetic construct
  REGION 1..8: note = Variant neuromedin B peptide
  MOD_RES 8: note = AMIDATION
GNLWATGH

SEQ ID NO: 27 - protein, 8 aa, synthetic construct
  REGION 1..8: note = Variant neuromedin B peptide
  MOD_RES 8: note = AMIDATION
NLWATGHF

SEQ ID NO: 28 - protein, 8 aa, synthetic construct
  REGION 1..8: note = Variant neuromedin B peptide
  MOD_RES 8: note = AMIDATION
LWATGHFM

SEQ ID NO: 29 - protein, 7 aa, synthetic construct
  REGION 1..7: note = Variant neuromedin B peptide
  MOD_RES 7: note = AMIDATION
GNLWATG

SEQ ID NO: 30 - protein, 7 aa, synthetic construct
  REGION 1..7: note = Variant neuromedin B peptide
  MOD_RES 7: note = AMIDATION
NLWATGH

SEQ ID NO: 31 - protein, 7 aa, synthetic construct
  REGION 1..7: note = Variant neuromedin B peptide
  MOD_RES 7: note = AMIDATION
LWATGHF

SEQ ID NO: 32 - protein, 7 aa, synthetic construct
  REGION 1..7: note = Variant neuromedin B peptide
  MOD_RES 7: note = AMIDATION
WATGHFM

SEQ ID NO: 33 - protein, 6 aa, synthetic construct
  REGION 1..6: note = Variant neuromedin B peptide
  MOD_RES 6: note = AMIDATION
GNLWAT

SEQ ID NO: 34 - protein, 6 aa, synthetic construct
  REGION 1..6: note = Variant neuromedin B peptide
  MOD_RES 6: note = AMIDATION
NLWATG

SEQ ID NO: 35 - protein, 6 aa, synthetic construct
  REGION 1..6: note = Variant neuromedin B peptide
  MOD_RES 6: note = AMIDATION
LWATGH

SEQ ID NO: 36 - protein, 6 aa, synthetic construct
  REGION 1..6: note = Variant neuromedin B peptide
  MOD_RES 6: note = AMIDATION
WATGHF

SEQ ID NO: 37 - protein, 6 aa, synthetic construct
  REGION 1..6: note = Variant neuromedin B peptide
  MOD_RES 6: note = AMIDATION
ATGHFM

SEQ ID NO: 38 - protein, 10 aa, Homo sapiens
GNHWAVGHLM

SEQ ID NO: 39 - protein, 18 aa, synthetic construct
  REGION 1..18: note = Cell penetrating peptide
RGGRLSYSRRRFSTSTGR

SEQ ID NO: 40 - protein, 10 aa, synthetic construct
  REGION 1..10: note = Cell penetrating peptide
RRLSYSRRRF

SEQ ID NO: 41 - protein, 12 aa, synthetic construct
  REGION 1..12: note = Cell penetrating peptide
PIRRRKKLRRLK

SEQ ID NO: 42 - protein, 12 aa, synthetic construct
  REGION 1..12: note = Cell penetrating peptide
RRQRRTSKLMKR

SEQ ID NO: 43 - protein, 15 aa, synthetic construct
  REGION 1..15: note = Cell penetrating peptide
RRRRNRTRRNRRRVR

SEQ ID NO: 44 - protein, 19 aa, synthetic construct
  REGION 1..19: note = Cell penetrating peptide
KMTRAQRRAAARRNRWTAR

SEQ ID NO: 45 - protein, 13 aa, synthetic construct
  REGION 1..13: note = Cell penetrating peptide
TRRQRTRRARRNR

SEQ ID NO: 46 - protein, 13 aa, synthetic construct
  REGION 1..13: note = Cell penetrating peptide
GRKKRRQRRRPPQ

SEQ ID NO: 47 - protein, 13 aa, synthetic construct
  REGION 1..13: note = Cell penetrating peptide
GRRRRRRRRRPPQ

SEQ ID NO: 48 - protein, 15 aa, synthetic construct
  REGION 1..15: note = Cell penetrating peptide
RQIKWFQNRRMKWKK

SEQ ID NO: 49 - protein, 17 aa, synthetic construct
  REGION 1..17: note = Cell penetrating peptide
KLALKLALKLALALKLA

SEQ ID NO: 50 - protein, 27 aa, synthetic construct
  REGION 1..27: note = Cell penetrating peptide
MGLGLHLLVLAAALQGAWSQPKKKRKV

SEQ ID NO: 51 - protein, 27 aa, synthetic construct
  REGION 1..27: note = Cell penetrating peptide
GALFLGWLGAAGSTMGAWSQPKKKRKV

SEQ ID NO: 52 - protein, 27 aa, synthetic construct
  REGION 1..27: note = Cell penetrating peptide
  SITE 1: note = MISC_FEATURE - Acetylation
  SITE 27: note = MISC_FEATURE - Cysteamide
GALFLGFLGAAGSTMGAWSQPKKKRKV

SEQ ID NO: 53 - protein, 27 aa, synthetic construct
  REGION 1..27: note = Cell penetrating peptide
  SITE 1: note = MISC_FEATURE - acetylation
  SITE 27: note = MISC_FEATURE - Cysteamide
GALFLGFLGAAGSTMGAWSQPKSKRKV

SEQ ID NO: 54 - protein, 21 aa, synthetic construct
  REGION 1..21: note = Cell penetrating peptide
  SITE 1: note = MISC_FEATURE - Acetylation
  SITE 21: note = MISC_FEATURE - Cysteamide
KETWFETWFTEWSQPKKKRKV

SEQ ID NO: 55 - protein, 27 aa, synthetic construct
  REGION 1..27: note = Cell penetrating peptide
GWTLNSAGYLLGKINLKALAALAKKIL

SEQ ID NO: 56 - protein, 21 aa, synthetic construct
  REGION 1..21: note = Cell penetrating peptide
  SITE 1: note = MISC_FEATURE - Acetylation
  SITE 21: note = MISC_FEATURE - Cysteamide
KETWWETWWTEWSQPKKKRKV

SEQ ID NO: 57 - protein, 11 aa, synthetic construct
  REGION 1..11: note = Cell penetrating peptide
YGRKKRRQRRR

SEQ ID NO: 58 - protein, 6 aa, synthetic construct
  REGION 1..6: note = Cell penetrating peptide
PFVYLI